SEQUENCE LISTING (58 sequences)

SEQ ID NO: 1 - DNA, 24 bp, Homo sapiens
agatatctcgcggtgctggggcca

SEQ ID NO: 2 - protein, 322 aa, Rattus norvegicus
MSSEAETQQPPAAPAAALSAADTKPGSTGSGAGSGGPGGLTSAAPAGGDKKVIATKVLGTVKWFNVRNGYGFINRNDTKEDVFVHQTAIKKNNPRKYLRSVGDGETVEFDVVEGEKGAEAANVTGPGGVPVQGSKYAADRNHYRRYPRRRGPPRNYQQNYQNSESGEKNEGSESAPEGQAQQRRPYRRRRFPPYYMRRPYARRPQYSNPPVQGEVMEGADNQGAGEQGRPVRQNMYRGYRPRFRRGPPRQRQPREDGNEEDKENQGDETQGQQPPQRRYRRNFNYRRRRPENPKPQDGKETKAADSPAENSSAPEAEQGGAE

SEQ ID NO: 3 - DNA, 1481 bp, Homo sapiens
ccgggagcggagagcggaccccagagagccctgagcagccccaccgccgccgccggcctagttaccatcacaccccgggaggagccgcagctgccgcagccggccccagtcaccatcaccgcaaccatgagcagcgaggccgagacccagcagccgcccgccgccccccccgccgcccccgccctcagcgccgccgacaccaagcccggcactacgggcagcggcgcagggagcggtggcccgggcggcctcacatcggcggcgcctgccggcggggacaagaaggtcatcgcaacgaaggttttgggaacagtaaaatggttcaatgtaaggaacggatatggtttcatcaacaggaatgacaccaaggaagatgtatttgtacaccagactgccataaagaagaataaccccaggaagtaccttcgcagtgtaggagatggagagactgtggagtttgatgttgttgaaggagaaaagggtgaggaggcagcaaatgttacaggtcctggtggtgttccagttcaaggcagtaaatatgcagcagaccgtaaccattatagacgctatccacgtcgtaggggtcctccacgcaattaccagcaaaattaccagaatagtgagagtggggaaaagaacgagggatcggagagtgctcccgaaggccaggcccaacaacgccggccctaccgcaggcgaaggttcccaccttactacatgcggagaccctatgggcgtcgaccacagtattccaaccctcctgtgcagggagaagtgatggagggtgctgacaaccagggtgcaggagaacaaggtagaccagtgaggcagaatatgtatcggggatatagaccacgattccgcaggggccctcctcgccaaagacagcctagagaggacggcaatgaagaagataaagaaaatcaaggagatgagacccaaggtcagcagccacctcaacgtcggtaccgccgcaacttcaattaccgacgcagacgcccagaaaaccctaaaccacaagatggcaaagagacaaaagcagccgatccaccagctgagaattcccgctcccgaggctgagcagggcggggctgagtaaatgccggcttaccatctctaccatcatccggtttagtcatccaacaagaagaaatatgaaattccagcaataagaaatgaacaaaagattggagctgaagacctaaagtacttgctttttgccgtttgcaaccagataaatagaactatctgcattatctatgcagcatggggtttatattttactaagacgctctttggtatacaacggttttaaaagcctggttttctcaatacgccttaaaggttttaaattgtttcatatctggtcaagttgagatttttaagaacttcatttttaatttgtaataaaagtttacaacttgattttttcaaaaaagtcaacaaactgcaagcacctgttaataaaggtcttaaataat

SEQ ID NO: 4 - protein, 317 aa, Homo sapiens
MSSEAETQQPPAAPPAAPALSAADTKPGTTGSGAGSGGPGGLTSAAPAGGDKKVIATKVLGTVKWFNVRNGYGFINRNDTKEDVFVHQTAIKKNNPRKYLRSVGDGETVEFDVVEGEKGEEAANVTGPGGVPVQGSKYAADRNHYRRYPRRRGPPRNYQQNYQNSESGEKNEGSESAPEGQAQQRRPYRRRRFPPYYMRRPYGRRPQYSNPPVQGEVMEGADNQGAGEQGRPVRQNMYRGYRPRFRRGPPRQRQPREDGNEEDKENQGDETQGQQPPQRRYRRNFNYRRRRPENPKPQDGKETKAADPPAENSRSRG

SEQ ID NO: 5 - protein, 10 aa, Unknown
  PTP catalytic domain
XHCXAGXXRX

SEQ ID NO: 6 - DNA, 42 bp, Artificial Sequence
  Nucleotide sequence within the PTP1B promoter and gene used for plasmid construction.
cgcgtacgcgcgctattagatatctcgcggtgctggggccac

SEQ ID NO: 7 - DNA, 42 bp, Artificial Sequence
  Nucleotide sequence within the PTP1B promoter and gene used for plasmid construction.
tcgagtggccccagcaccgcgagatatctaatagcgcgcgta

SEQ ID NO: 8 - DNA, 30 bp, Artificial Sequence
  Nucleotide sequence within the PTP1B promoter and gene used for plasmid construction.
cgcgtacgcgcgctattagatatctcgcgc

SEQ ID NO: 9 - DNA, 30 bp, Artificial Sequence
  Nucleotide sequence within the PTP1B promoter and gene used for plasmid construction.
tcgagcgcgagatatctaatagcgcgcgta

SEQ ID NO: 10 - DNA, 17 bp, Artificial Sequence
  Nucleotide sequence within the PTP1B promoter and gene used for plasmid construction.
cgcgtacgcgcgctatt

SEQ ID NO: 11 - DNA, 18 bp, Artificial Sequence
  Nucleotide sequence within the PTP1B promoter and gene used for plasmid construction.
tcgagaatagcgcgcgta

SEQ ID NO: 12 - DNA, 30 bp, Artificial Sequence
  Nucleotide sequence within the PTP1B promoter and gene used for plasmid construction.
cgcgtagatatctcgcggtgctggggccac

SEQ ID NO: 13 - DNA, 30 bp, Artificial Sequence
  Nucleotide sequence within the PTP1B promoter and gene used for plasmid construction.
tcgagtggccccagcaccgcgagatatcta

SEQ ID NO: 14 - DNA, 18 bp, Artificial Sequence
  Nucleotide sequence within the PTP1B promoter and gene used for plasmid construction.
cgcgtagatatctcgcgc

SEQ ID NO: 15 - DNA, 18 bp, Artificial Sequence
  Nucleotide sequence within the PTP1B promoter and gene used for plasmid construction.
tcgagcgcgagatatcta

SEQ ID NO: 16 - DNA, 18 bp, Artificial Sequence
  Nucleotide sequence within the PTP1B promoter and gene used for plasmid construction.
cgcgtgtgctggggccac

SEQ ID NO: 17 - DNA, 30 bp, Artificial Sequence; identical to SEQ ID NO: 13
  Nucleotide sequence within the PTP1B promoter and gene used for plasmid construction.

SEQ ID NO: 18 - DNA, 31 bp, Artificial Sequence
  Nucleotide sequence within the PTP1B promoter and gene used for plasmid construction.
cgcgtaacgcgcgctattgtgctggggccac

SEQ ID NO: 19 - DNA, 18 bp, Artificial Sequence
  Nucleotide sequence within the PTP1B promoter and gene used for plasmid construction.
tcgagtggccccagcaca

SEQ ID NO: 20 - DNA, 20 bp, Artificial Sequence
  Oligonucleotide used as 5′ primer for making deletion mutants
ggtaccgagctcttacgcgt

SEQ ID NO: 21 - DNA, 36 bp, Artificial Sequence
  Oligonucleotide used as 5′ primer for making deletion mutants
actatagggcacgcgtacgcgcgctattagatatct

SEQ ID NO: 22 - DNA, 35 bp, Artificial Sequence
  Oligonucleotide used as 5′ primer for making deletion mutants
actatagggcacgcgtatctcgcggtgctggggcc

SEQ ID NO: 23 - DNA, 39 bp, Artificial Sequence
  Oligonucleotide used as 3′ primer for making deletion mutants
cccctcgaggacgggccagggcggctgctgcgcctcctt

SEQ ID NO: 24 - DNA, 36 bp, Artificial Sequence
  Oligonucleotide labeled at 5′ end and used as probes for an electrophoretic mobility shift assay
acgcgcgctattagatatctcgcggtgctggggcca

SEQ ID NO: 25 - DNA, 36 bp, Artificial Sequence
  Oligonucleotide labeled at 5′ end and used as probes for an electrophoretic mobility shift assay
tggccccagcaccgcgagatatctaatagcgcgcgt

SEQ ID NO: 26 - DNA, 24 bp, Artificial Sequence
  Oligonucleotide labeled at 5′ end and used as probes for an electrophoretic mobility shift assay
acgcgcgctattagatatctcgcg

SEQ ID NO: 27 - DNA, 24 bp, Artificial Sequence
  Oligonucleotide labeled at 5′ end and used as probes for an electrophoretic mobility shift assay
cgcgagatatctaatagcgcgcgt

SEQ ID NO: 28 - DNA, 24 bp, Artificial Sequence
  Oligonucleotide labeled at 5′ end and used as probes for an electrophoretic mobility shift assay
agatatctcgcggtgctggggcca

SEQ ID NO: 29 - DNA, 24 bp, Artificial Sequence
  Oligonucleotide labeled at 5′ end and used as probes for an electrophoretic mobility shift assay
tggccccagcaccgcgagatatct

SEQ ID NO: 30 - DNA, 24 bp, Artificial Sequence
  Oligonucleotide labeled at 5′ end and used as probes for an electrophoretic mobility shift assay
acgcgcgctattgtgctggggcca

SEQ ID NO: 31 - DNA, 24 bp, Artificial Sequence
  Oligonucleotide labeled at 5′ end and used as probes for an electrophoretic mobility shift assay
tggccccagcacaatagcgcgcgt

SEQ ID NO: 32 - DNA, 36 bp, Artificial Sequence; identical to SEQ ID NO: 24
  Sequence used for affinity chromatography corresponding to nucleotides -167 to -132 form the transcription start site.

SEQ ID NO: 33 - DNA, 36 bp, Artificial Sequence; identical to SEQ ID NO: 25
  Sequence used for affinity chromatography corresponding to nucleotides -167 to -132 form the transcription start site.

SEQ ID NO: 34 - DNA, 39 bp, Artificial Sequence; identical to SEQ ID NO: 23
  3′ primer sequence for making deletion mutants

SEQ ID NO: 35 - protein, 8 aa, Artificial Sequence
  pCMV-tag empty vector control.
DYKDDDDK

SEQ ID NO: 36 - DNA, 24 bp, Artificial Sequence
  PCR primer
ttgggaacagtaaaatggttcaat

SEQ ID NO: 37 - DNA, 24 bp, Artificial Sequence
  PCR primer
ctgcttctgtctctttgccatctt

SEQ ID NO: 38 - DNA, 24 bp, Artificial Sequence
  PCR primer
atggagatggaaaaggagttcgag

SEQ ID NO: 39 - DNA, 24 bp, Artificial Sequence
  PCR primer
gatatactcattatcttcttgatg

SEQ ID NO: 40 - DNA, 24 bp, Artificial Sequence
  antisense oligonucleotide
agttatctcgcggtgctggggcca

SEQ ID NO: 41 - DNA, 24 bp, Artificial Sequence
  antisense oligonucleotide
agatatctcgcggtgctggcgcca

SEQ ID NO: 42 - DNA, 24 bp, Artificial Sequence
  antisense oligonucleotide
agatatctcgcggagctgggccca

SEQ ID NO: 43 - DNA, 20 bp, Artificial Sequence
  antisense oligonucleotide
agatatctcgcggtgctggg

SEQ ID NO: 44 - DNA, 21 bp, Artificial Sequence
  antisense oligonucleotide
agatatctcgcggtgctgggg

SEQ ID NO: 45 - DNA, 22 bp, Artificial Sequence
  antisense oligonucleotide
agatatctcgcggtgctggggc

SEQ ID NO: 46 - DNA, 23 bp, Artificial Sequence
  antisense oligonucleotide
agatatctcgcggtgctggggcc

SEQ ID NO: 47 - DNA, 20 bp, Artificial Sequence
  antisense oligonucleotide
atctcgcggtgctggggcca

SEQ ID NO: 48 - DNA, 21 bp, Artificial Sequence
  antisense oligonucleotide
tatctcgcggtgctggggcca

SEQ ID NO: 49 - DNA, 22 bp, Artificial Sequence
  antisense oligonucleotide
atatctcgcggtgctggggcca

SEQ ID NO: 50 - DNA, 23 bp, Artificial Sequence
  antisense oligonucleotide
gatatctcgcggtgctggggcca

SEQ ID NO: 51 - protein, 322 aa, Rattus norvegicus; identical to SEQ ID NO: 2

SEQ ID NO: 52 - DNA, 1489 bp, Rattus norvegicus
gaattccgagagccccaccgccgccgccggcctagtcaccatcacacccgggaggagccgcagccgtcgccgccggccccagtcaccatcaccgcaaccatgagcagcgaggccgagacccagcagccgcccgccgcccccgccgccgccctcagcgccgccgacaccaagcccggctccacgggcagcggcgcgggtagtggcggcccgggcggcctcacatcggcggcgcccgccggcggggacaagaaggtcatcgcaacgaaggttttgggaacagtaaaatggttcaatgtaaggaacggatacggtttcatcaacaggaatgacaccaaggaagacgtatttgtacaccagacggccataaagaagaataaccccaggaagtaccttcgcagtgtaggagatggagagactgtggagtttgatgttgttgaaggagaaaagggtgcggaggcagctaatgttacaggccctggtggagttccagttcaaggcagtaaatacgcagcagaccgtaaccattataggcgctatccacgtcgtaggggtcctccacgcaattaccagcaaaattaccagaatagtgagagtggggaaaagaatgaaggatcggaaagcgctcctgaaggccaggcccaacaacgccggccctatcgcaggcgaaggttcccaccttactacatgcggagaccctatgcgcgtcgaccacagtattccaacccccctgtgcaaggagaagtgatggagggtgctgacaaccagggtgcaggagagcaaggtagaccagtgagacagaatatgtatcggggttacagaccacgattccgcaggggccctcctcgccaaagacagcctagagaggatggcaatgaagaggacaaagaaaatcaaggagatgagacccaaggtcagcagccacctcaacgtcggtatcgccgcaacttcaattaccgacgcagacgcccagagaaccctaaaccacaagatggcaaagagacaaaagcagccgattcaccagctgagaattcgtccgctcccgaggctgagcagggcggggctgagtaaatgccggcttaccatctctaccatcatccggtttggtcatccaacaagaagaaatgaatatgaaattccagcaataagaaatgaacaaagattggagctgaagaccttaagtgcttgctttttgcccgttgaccagatccactagaactgtctgcattatctatgcagcatggggtttttattatttttacctaaagatgtctctttttggtaatgacaaacgtgttttttaagaaaaaaaaaaaaaaggcctggtttttctcaatacacctttaacggtttttaaattgtttcatatctggtcaagttgagatttttaagaacgttcgatttttaatttgtaataaagtttacaacttgattttttcaaaaaagtcaacaaactgcaagcacctgttaataaaggtcttaaatggaattc

SEQ ID NO: 53 - protein, 322 aa, Rattus norvegicus; identical to SEQ ID NO: 2

SEQ ID NO: 54 - protein, 1477 aa, Saccharomyces cerevisiae
MTITVGDAVSETELENKSQNVVLSPKASASSDISTDVDKDTSSSWDDKSLLPTGEYIVDRNKPQTYLNSDDIEKVTESDIFPQKRLFSFLHSKKIPEVPQTDDERKIYPLFHTNIISNMFFWWVLPILRVGYKRTIQPNDLFKMDPRMSIETLYDDFEKNMIYYFEKTRKKYRKRHPEATEEEVMENAKLPKHTVLRALLFTFKKQYFMSIVFAILANCTSGFNPMITKRLIEFVEEKAIFHSMHVNKGIGYAIGACLMMFVNGLTFNHFFHTSQLTGVQAKSILTKAAMKKMFNASNYARHCFPNGKVTSFVTTDLARIEFALSFQPFLAGFPAILAICIVLLIVNLGPIALVGIGIFFGGFFISLFAFKLILGFRIAANIFTDARVTMMREVLNNIKMIKYYTWEDAYEKNIQDIRTKEISKVRKMQLSRNFLIAMAMSLPSIASLVTFLAMYKVNKGGRQPGNIFASLSLFQVLSLQMFFLPIAIGTGIDMIIGLGRLQSLLEAPEDDPNQMIEMKPSPGFDPKLALKMTHCSFEWEDYELNDAIEEAKGEAKDEGKKNKKKRKDTWGKPSASTNKAKRLDNMLKDRDGPEDLEKTSFRGFKDLNFDIKKGEFIMITGPIGTGKSSLLNAMAGSMRKTDGKVEVNGDLLMCGYPWIQNASVRDNIIFGSPFNKEKYDEVVRVCSLKADLDILPAGDMTEIGERGITLSGGQKARINLARSVYKKKDIYLFDDVLSAVDSRVGKHIMDECLTGMLANKTRILATHQLSLIERASRVIVLGTDGQVDIGTVDELKARNQTLINLLQFSSQNSEKEDEEQEAVVAGELGQLKYESEVKELTELKKKATEMSQTANSGKIVADGHTSSKEERAVNSISLKIYREYIKAAVGKWGFIALPLYAILVVGTTFCSLFSSVWLSYWTENKFKNRPPSFYMGLYSFFVFAAFIFMNGQFTILCAMGIMASKWLNLRAVKRILHTPMSYIDTTPLGRILNRFTKDTDSLDNELTESLRLMTSQFANIVGVCVMCIVYLPWFAIAIPFLLVIFVLIADHYQSSGREIKRLEAVQRSFVYNNLNEVLGGMDTIKAYRSQERFLAKSDFLINKMNEAGYLVVVLQRWVGIFLDMVAIAFALIITLLCVTRAFPISAASVGVLLTYVLQLPGLLNTILRAMTQTENDMNSAERLVTYATELPLEASYRKPEMTPPESWPSMGEIIFENVDFAYRPGLPIVLKNLNLNIKSGEKIGICGRTGAGKSTIMSALYRLNELTAGKILIDNVDISQLGLFDLRRKLAIIPQDPVLFRGTIRKNLDPFNERTDDELWDALVRGGAIAKDDLPEVKLQKPDENGTHGKMHKFHLDQAVEEEGSNFSLGERQLLALTRALVRQSKILILDEATSSVDYETDGKIQTRIVEEFGDCTILCIAHRLKTIVNYDRILVLEKGEVAEFDTPWTLFSQEDSIFRSMCSRSGIVENDFENRS

SEQ ID NO: 55 - protein, 322 aa, Mus musculus
MSSEAETQQPPAAPAAALSAADTKPGSTGSGAGSGGPGGLTSAAPAGGDKKVIATKVLGTVKWFNVRNGYGFINRNDTKEDVFVHQTAIKKNNPRKYLRSVGDGETVEFDVVEGEKGAEAANVTGPGGVPVQGSKYAADRNHYRRYPRRRGPPRNYQQNYQNSESGEKNEGSESAPEGQAQQRRPYRRRRFPPCYMRRPYARRPQYSNPPVQGEVMEGADNQGAGEQGRPVRQNMYWGYRPRFRRGPPRQRQPREDGNEEDKENQGDETQGQQPPQRRYRRNFNYRRRRPENPKPQDGKETKAADPPAENSSAPEAEQGGAE

SEQ ID NO: 56 - protein, 322 aa, Mus musculus
MSSEAETQQPPAAPAAALSAADTKPGSTASGAGSGGPGGLTSAAPAGGDKKVIATKVLGTVKWFNVRNGYGFINRNDTKEDVFVHQTAIKKNNPRKYLRSVGDGETVEFDVVEGEKGAEAANVTGPGGVPVQGSKYAADRNHYRRYPRRRGPPRNYQQNYQNSESGEKNEGSESAPEGQAQQRRPYRRRRFPPYYMRRPYARRPQYSNPPVQGEVMEGADNQGAGEQGRPVRQNMYRGYRPRFRRGPPRQRQPREDGNEEDKENQGDETQGQQPPQRRYRRNFNYRRRRPENPKPQDGKETKAADPPAENSSAPEAEQGGAE

SEQ ID NO: 57 - protein, 322 aa, Rattus norvegicus
MSSEAETQQPPAAPAAALSAADTKPGSTGSGAGSGGPGGLTSAAPAGGDKKVIATKVLGTVKWFNVRNGYGFINRNDTKEDVFVHQTAIKKNNPRKYLRSVGDGETVEFDVVEGEKGAEAANVTGPGGVPVQGSKYAADRNHYRRYPRRRGPPRNYQQNYQNSESGEKNEGSESAPEGQAQQRRPYRRRRFPPYYMRRPYARRPQYSNPPVQGEVMEGADNQGAGEQGRPVRQNMYRGYRPRFRRGPPRQRQPREDGNEEDKENQGDETQGQQPPQRRYRRNFNYRRRRPENPKPQDGKETKAADPPAENSSAPEAEQGGAE

SEQ ID NO: 58 - protein, 322 aa, Rattus norvegicus
MSSEAETQQPPAAPAAALSAADTKPGSTGSGAGSGGPGGLTSAAPAGGDKKVIATKVLGTVKWFNVRNGYGFINRNDTKEDVFVHQTAIKKNNPRKYLRSVGDGETVEFDVVEGEKGAEAANVTGPGGVPVQGSKYAADRNHYRRYPRRRGPPRNYQQNYQNSESGEKNEGSESAPEGQAQQRGPYRSRRFPPYYMRRPYARRPQYSNPPVQGEVMEGADNQGAGEQGRPVRQNMYRGYRPRFRRGPPRPRQPREDGNEEDKENQGDETQGQQPPQRRYRRNFNYRRRRPENPKPQDGKETKAADPPAENSSAPEAEQGGAE

(19) United States
(12) Patent Application Publication
(10) Pub. No.: US 2003/0223975 A1
(43) Pub. Date: Dec. 4, 2003

(54) Transcriptional regulation of PTP-1B

(75) Inventors: Nicholas K. Tonks, Huntington, NY (US); Toshiyuki Fukada, Huntington, NY (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Cold Spring Harbor Laboratory, Cold Spring Harbor, NY (US)

(21) Appl. No.: 10/388,215

(22) Filed: Mar. 11, 2003

Related U.S. Application Data
(60) Provisional application No. 60/363,787, filed on Mar. 12, 2002.
(60) Provisional application No. 60/435,587, filed on Dec. 20, 2002.

(51) Int. Cl. A61K 48/00; C12Q 1/68; C07H 21/04; C12N 9/16; C12P 21/02; C12N 5/06

(52) U.S. Cl. 424/93.21; 514/44; 435/6; 435/320.1; 435/325; 435/196; 435/69.1; 536/23.2

(57) ABSTRACT

Compositions and methods relating to PTP1B associated disorders are provided, based on the discovery that a Y-box protein binding site is present as a transcription enhancer sequence in the promoter region situated upstream (i.e., 5′ to) of the human PTP1B gene. This site, situated at nucleotides −155 through −132 of the human PTP1B gene, mediates specific binding interactions with the YB-1 transcription regulatory factor, a member of the Y-box family of proteins. YB-1-targeted antisense constructs reduced PTP1B expression levels, providing an alternative to PTP1B active-site directed regulation of PTP1B activity.

### CROSS-REFERENCES TO RELATED APPLICATIONS

This application claims the benefit of U.S. Provisional Patent Application No. 60/363,787 filed Mar. 12, 2002, and No. 60/435,587 filed Dec. 20, 2002, which are incorporated herein by reference in their entirety.

### STATEMENT REGARDING FEDERALLY SPONSORED RESEARCH

This invention was made in part with government support under Grant Nos. CA53840 and P30CA45508 awarded by the National Institutes of Health. The government may have certain rights in this invention.

### FIELD OF THE INVENTION

The present invention relates generally to compositions and methods useful for treating conditions associated with defects in biological signal transduction in cells. More specifically, the invention relates to treatment of diabetes, obesity, impaired glucose tolerance and the like, and other metabolic disorders, by intervening in the transcriptional regulation of PTP1B expression, to screening assays for agents that alter the mechanism of such regulation, and to related compositions and methods.

### DESCRIPTION OF THE RELATED ART

Reversible protein tyrosine phosphorylation, coordinated by the action of protein tyrosine kinases (PTKs) that phosphorylate certain tyrosine residues in polypeptides, and protein tyrosine phosphatases (PTPs) that dephosphorylate certain phosphotyrosine residues, is a key mechanism in regulating many cellular activities. The diversity and complexity of the PTPs and PTKs are comparable; PTPs are equally important in delivering both positive and negative signals for proper function of cellular machinery. Regulated tyrosine phosphorylation contributes to specific pathways for biological signal transduction, including those associated with cell division, proliferation, and differentiation. Defects and malfunctions in these pathways may underlie certain disease conditions for which effective intervention remains elusive, including for example, metabolic disorders and conditions, such as diabetes and obesity, malignancy, infection, and immune disorders including those characterized by inappropriate or undesirable immunosuppression, inflammation, or autoimmunity. Selective regulation of PTPs may represent a useful treatment strategy for these and other diseases.

The protein tyrosine phosphatase (PTP) family of enzymes consists of more than 500 structurally diverse proteins that have in common the highly conserved 250 amino acid PTP catalytic domain, but which display considerable variation in their non-catalytic segments (Charbonneau and Tonks, Annu. Rev. Cell Biol. 8:463-93 (1992); Tonks, Semin. Cell Biol. 4:373-453 (1993)). This structural diversity of individual PTP family members presumably reflects the diversity of their physiological roles, which in certain cases includes specific functions in growth, development, and differentiation (Desai et al., Cell 84:599-609 (1996); Kishihara et al., Cell 74:143-56 (1993); Perkins et al., Cell 70:225-36 (1992); Pingel and Thomas, Cell 58:1055-65 (1989); Schultz et al., Cell 73:1445-54 (1993)).

The PTP family of enzymes contains a common evolutionarily conserved segment of approximately 250 amino acids known as the PTP catalytic domain. Within this conserved domain is a unique signature sequence motif,

[I/V]HCXAGXXR[S/T] SEQ ID NO:5,

that is invariant among all PTPs with the exception of certain members of the PTP subfamily known as dual specificity phosphatases (DSPs), in which the alanine and the serine residues in SEQ ID NO:5 may not be conserved. The cysteine residue in this motif is invariant in members of the PTP family and is essential for catalysis of the phosphotyrosine dephosphorylation reaction. This invariant cysteine functions as a nucleophile to attack the phosphate moiety present on a phosphotyrosine residue of the incoming substrate.

The PTP family consists of receptor-like and non-transmembrane enzymes that exhibit exquisite substrate specificity in vivo and that are critical regulators of a wide variety of cellular signaling pathways (Andersen et al., Mol. Cell. BioL 21:7117 (2001); Tonks and Neel, Curr. Opin. Cell Biol. 13:182 (2001)). One non-transmembrane PTP, PTP-1B (PTP1B), recognizes several tyrosine phosphorylated proteins as substrates, many of which are involved in human disease. For example, PTP1B acts as a negative regulator of signaling that is initiated by several PTKs, including p210 Bcr-Abl (LaMontagne et al., Mol. Cell. Biol. 18:2965-75 (1998); LaMontagne et al., Proc. Natl. Acad. Sci. USA 95:14094-99 (1998)), receptor tyrosine kinases, such as EGF receptor, PDGF receptor, and insulin receptor (IR) (Tonks et al., Curr. Opin. Cell Biol. 13:182-95 (2001)), and JAK family members (Myers et al., J Biol. Chem. 276:47771-74 (2001)). PTPs participate in a variety of physiologic functions, providing a number of opportunities for therapeutic intervention in physiologic processes through alteration (i.e., a statistically significant increase or decrease) or modulation (e.g., up-regulation or down-regulation) of PTP activity.

PTP1B has been reported to act as a negative regulator of signaling events initiated by several growth factor/hormone receptor PTKs, as well as signaling events induced by cytokines (Tonks and Neel, 2001). Activity of PTP1B is regulated by modifications of several amino acid residues, such as phosphorylation of Ser residues (Brautigan and Pinault, 1993; Dadke et al., 2001; Flint et al., 1993), and oxidation of the active Cys residue in its catalytic motif (Lee et al., J. Biol. Chem. 273:15366-72 (1998); Meng et al., Mol. Cell 9:387-99 (2002)) which is evolutionary conserved among protein tyrosine phosphatases and dual phosphatase family members (Andersen et al., 2001). In addition, changes in the expression levels of PTP1B have been noted in several human diseases, particularly those associated with disruption of the normal patterns of tyrosine phosphorylation. For example, therapeutic inhibition of PTPs such as PTP1B in the insulin signaling pathway may serve to augment insulin action, thereby ameliorating the state of insulin resistance common in patients with type 2 diabetes.

Diabetes mellitus is a common, degenerative disease affecting 5-10% of the human population in developed countries, and in many countries, it may be one of the five leading causes of death. Approximately 2% of the world's population has diabetes, the overwhelming majority of cases (>97%) being type 2 diabetes and the remainder being type 1. In type I diabetes, which is frequently diagnosed in children or young adults, insulin production by pancreatic islet beta cells is destroyed. Type 2 diabetes, or “late onset” or “adult onset” diabetes, is a complex metabolic disorder in which cells and tissues cannot effectively use available insulin; in some cases insulin production is also inadequate. At the cellular level, the degenerative phenotype that may be characteristic of late onset diabetes mellitus includes, for example, impaired insulin secretion and decreased insulin sensitivity, i.e., an impaired response to insulin.

Studies have shown that diabetes mellitus may be preceded by or is associated with certain related disorders. For example, an estimated forty million individuals in the U.S. suffer from late onset impaired glucose tolerance (IGT). IGT patients fail to respond to glucose with increased insulin secretion. Each year a small percentage (5-10%) of IGT individuals progress to insulin deficient non-insulin dependent diabetes (NIDDM). Some of these individuals further progress to insulin dependent diabetes mellitus (IDDM). NIDDM and IDDM are associated with decreased release of insulin by pancreatic beta cells and/or a decreased response to insulin by cells and tissues that normally exhibit insulin sensitivity. Other symptoms of diabetes mellitus and conditions that precede or are associated with diabetes mellitus include obesity, vascular pathologies, and various neuropathies, including blindness and deafness.

Type 1 diabetes is treated with lifelong insulin therapy, which is often associated with undesirable side-effects such as weight gain and an increased risk of hypoglycemia. Current therapies for type 2 diabetes (NIDDM) include altered diet, exercise therapy, and pharmacological intervention with injected insulin or oral agents that are designed to lower blood glucose levels. Examples of such presently available oral agents include sulfonylureas, biguanides, thiazolidinediones, repaglinide, and acarbose, each of which alters insulin and/or glucose levels. None of the current pharmacological therapies, however, controls the disease over its full course, nor do any of the current therapies correct all of the physiological abnormalities in type 2 NIDDM, such as impaired insulin secretion, insulin resistance, and excessive hepatic glucose output. In addition, treatment failures are common with these agents, such that multi-drug therapy is frequently necessary.

In certain metabolic diseases or disorders, one or more biochemical processes, which may be either anabolic or catabolic (e.g., build-up or breakdown of substances, respectively), are altered (e.g., increased or decreased in a statistically significant manner) relative to the levels at which they occur in a disease-free or normal subject such as an appropriate control individual. The alteration may result from an increase or decrease in a substrate, enzyme, cofactor, or any other component in any biochemical reaction involved in a particular process. Altered (i.e., increased or decreased in a statistically significant manner relative to a normal state) PTP activity can underlie certain disorders and suggests a PTP role in certain metabolic diseases. For example, disruption of the murine PTP1B gene homolog in a knock-out mouse model results in PTP1B^{-/-} mice exhibiting enhanced insulin sensitivity, decreased levels of circulating insulin and glucose, and resistance to weight gain even on a high-fat diet, relative to control animals having at least one functional PTP1B gene (Elchebly et al., Science 283:1544 (1999)). Insulin receptor hyperphosphorylation has also been detected in certain tissues of PTP1B deficient mice, consistent with a PTP1B contribution to the physiologic regulation of insulin and glucose metabolism (Id.; see also Klaman et al., Mol. Cell Biol. 20:5479-89 (2000)). Additionally, altered PTP activity has been correlated with impaired glucose metabolism in other biological systems (e.g., McGuire et al., Diabetes 40:939 (1991); Myerovitch et al., J. Clin. Invest. 84:976 (1989); Sredy et al. Metabolism 44:1074 (1995)), including PTP involvement in biological signal transduction via the insulin receptor (see, e.g., WO 99/46268 and references cited therein) (See also, e.g., Ahmad et al., Metabolism 46:1140-45 (1997); Bleyle et al., Cell Signal 11:719-25 (1999); Echwald et al., Diabetes 51:1-6 (2002); Di Paola et al., Am. J. Hum. Genet. 70:806-12 (2002); Kenner et al., J. Biol. Chem. 268:25455-62 (1993); Kenner et al., J. Biol. Chem. 271:19810-16 (1996); Klupa et al., Diabetes 49:2212-16 (2000); Lee et al., Am. J. Hum. Genet. 64:196-209 (1999); Lembertas et al., J. Clin. Invest. 100:1240-47 (1997); Mok et al., J. Clin. Endocrinol. Metab. 87:724-27 (2002); Tao et al., J. Biol. Chem. 276:39705-12 (2001)).

An integration of crystallographic, kinetic, and PTP1B-peptide binding assays illustrated the interaction of PTP1B and IR (Salmeen et al., Mol. Cell 6:1401-12 (2000)). The insulin receptor (IR) comprises two extracellular a subunits and two transmembrane β subunits. Activation of the receptor results in autophosphorylation of tyrosine residues in both β subunits, each of which contains a protein kinase domain. Extensive interactions that form between PTP1B and insulin receptor kinase (IRK) encompass tandem pTyr residues at 1162 and 1163 of IRK, such that pTyr-1162 is located in the active site of PTP1B (id.). The Asp/Glu-pTyr-pTyr-Arg/Lys motif has been implicated for optimal recognition by PTP1B for IRK. This motif is also present in other receptor PTKs, including Trk, FGFR, and Axl. In addition, this motif is found in the JAK family of PTKs, members of which transmit signals from cytokine receptors, including a classic cytokine receptor that is recognized by the satiety hormone leptin (Touw et al., Mol. Cell. Endocrinol. 160:1-9 (2000)).

Changes in the expression levels of PTP1B have been observed in several human diseases, particularly in diseases associated with disruption of the normal patterns of tyrosine phosphorylation. For example, the expression of PTP1B is induced specifically by the p210 Bcr-Abl oncoprotein, a PTK that is directly responsible for the initial manifestations of chronic myelogenous leukemia (CML) (LaMontagne et al., Mol. Cell. Biol. 18:2965-75 (1998); LaMontagne et al., Proc. Natl. Acad. Sci. USA 95:14094-99 (1998)). Expression of PTPB1 in response to this oncoprotein is regulated, in part, by transcription factors Sp1, Sp3, and Egr-1 (Fukada et al., J. Biol. Chem. 276:25512-19 (2001)). These transcription factors have been shown to bind to a p210 Bcr-Abl responsive sequence (PRS) in the human PTP1B promoter, located between −49 to −37 base pairs from the transcription start site, but do not appear to mediate certain additional, independent PTP1B transcriptional events, for which neither transcription factor(s) nor transcription factor recognition element(s) have been defined (id.). (See also, e.g., Wiener et al., Am. J. Obstet. Gynecol. 170:1177-83 (1994); Wieneret al., J. Natl. Cancer Inst. 86:372-78 (1994); Zhai et al., Cancer Res. 53:2272-78 (1993)).

Currently, therefore, desirable goals for determining the molecular mechanisms that govern PTP1B-mediated cellular events include, inter alia, identification of PTP interacting molecules, substrates and binding partners, and agents that regulate PTP activities. Accordingly, a need exists in the art for an improved ability to intervene in the regulation of phosphotyrosine signaling, including regulating PTPs by altering PTP catalytic activity, PTP binding to PTP substrate molecules, and/or PTP-encoding gene expression. An increased ability to so regulate PTPs may facilitate the development of methods for modulating the activity of proteins involved in phosphotyrosine signaling pathways and for treating conditions associated with such pathways. The present invention fulfills these needs and further provides other related advantages.

### BRIEF SUMMARY OF THE INVENTION

It is an aspect of the present invention to provide an isolated polynucleotide comprising a nucleotide sequence as set forth in SEQ ID NO:1 or a variant thereof, or a complementary sequence thereto. In certain embodiments there is provided an isolated antisense polynucleotide comprising at least 20 consecutive nucleotides complementary to a polynucleotide as set forth in SEQ ID NO:1. In certain embodiments there is provided an isolated polynucleotide comprising at least 15 consecutive nucleotides that is capable of hybridizing under moderately stringent conditions to a nucleotide sequence as set forth in SEQ ID NO:1 [AGATATCTCG CGGTGCTGGG GCCA], or a complementary sequence thereto. In certain embodiments the invention provides a recombinant nucleic acid construct comprising a polynucleotide as just described. In another embodiment the invention provides a recombinant nucleic acid construct comprising a nucleotide sequence as set forth in SEQ ID NO:1 [AGATATCTCG CGGTGCTGGG GCCA] that is operably linked to a reporter gene, which in certain further embodiments encodes a luciferase or a chloramphenicol acetyl transferase polypeptide. Also provided are vectors comprising any of the foregoing recombinant nucleic acid constructs, and host cells transformed or transfected with such vectors.

In another embodiment the invention provides a method for treating a PTP1B associated disorder comprising administering to a subject an agent that impairs binding of a Y-box protein to a PTP1B promoter Y-box protein binding site. In one embodiment the PTP1B associated disorder is a metabolic disorder and in another embodiment the disorder is type 1 diabetes, type 2 diabetes, obesity or impaired glucose tolerance. In certain embodiments of the invention, the agent that impairs binding of a Y-box protein to a PTP1B promoter Y-box protein binding site comprises an isolated polynucleotide comprising a nucleotide sequence set forth in SEQ ID NO:1 or a variant thereof, or a complementary sequence thereto; an isolated antisense polynucleotide comprising at least 20 consecutive nucleotides complementary to a nucleotide sequence set forth in SEQ ID NO:1 or to a variant thereof; or an isolated polynucleotide comprising at least 15 consecutive nucleotides that is capable of hybridizing under moderately stringent conditions to a nucleotide sequence set forth in SEQ ID NO:1. In one embodiment the agent that impairs binding of a Y-box protein to a PTP1B promoter Y-box protein binding site comprises an antisense polynucleotide, the antisense polynucleotide comprising at least 15 consecutive nucleotides complementary to a polynucleotide that encodes a YB-1 protein. In a further embodiment the YB-1 protein comprises an amino acid sequence as set forth in FIG. 3D and SEQ ID NO:2. In another further embodiment the YB-1 protein comprises an amino acid sequence as set forth in, or encoded by, a sequence selected from GENBANK Accession No. J03827 [SEQ ID NO:4], GENBANK Accession No. NP_{—}113751 [SEQ ID NO:51], GENBANK Accession No.M57299 [SEQ ID NO:52; SEQ ID NO:53], GENBANK Accession No. AAB46889.2 [SEQ ID NO:58], GENBANK Accession No. AAA35750.1 [SEQ ID NO:54], GENBANK Accession No. AAA75476.1 [SEQ ID NO:55], GENBANK Accession No. AAA63390.1 [SEQ ID NO:56], and GENBANK Accession No. BAA02569.1 [SEQ ID NO:57]. In another embodiment the antisense polynucleotide comprises at least 20 consecutive nucleotides of a polynucleotide selected from a polynucleotide having a sequence as set forth in, or capable of encoding, SEQ ID NO:3, GENBANK Accession No. J03827 [SEQ ID NO:4], GENBANK Accession No. NP_{—}113751 [SEQ ID NO:51], GENBANK Accession No.M57299 [SEQ ID NO:52; SEQ ID NO:53], GENBANK Accession No. AAB46889.2 [SEQ ID NO:58], GENBANK Accession No. AAA35750.1 [SEQ ID NO:54], GENBANK Accession No. AAA75476.1 [SEQ ID NO:55], GENBANK Accession No. AAA63390.1 [SEQ ID NO:56], and GENBANK Accession No. BAA02569.1 [SEQ ID NO:57], or a complementary sequence thereto.

In other embodiments the agent that impairs binding of a Y-box protein to a PTP1B promoter Y-box protein binding site comprises a polypeptide, which in certain further embodiments comprises an antibody that specifically binds to the Y-box protein. In certain embodiments of the invention, the antibody is an intracellular antibody. In another embodiment the agent that impairs binding of a Y-box protein to a PTP1B promoter Y-box protein binding site is a small molecule. In other embodiments the PTP1B promoter Y-box protein binding site comprises a polynucleotide having a nucleotide sequence as set forth in SEQ ID NO:1 or a complement thereto. In another embodiment the PTP1B promoter Y-box protein binding site comprises a polynucleotide that is capable of hybridizing under moderately stringent conditions to a nucleotide sequence as set forth in SEQ ID NO:1, or a complementary sequence thereto.

The invention also provides a method for treating a PTP1B associated disorder comprising administering to a subject a pharmaceutical compositions that comprises an agent which impairs binding of a Y-box protein to a PTP1B promoter Y-box protein binding site and a suitable carrier. In another embodiment, the invention provides a method for impairing binding of a Y-box polypeptide to a PTP1B promoter Y-box polypeptide binding site, comprising contacting a cell which comprises a Y-box polypeptide and a PTP1B promoter Y-box polypeptide binding site with a polynucleotide comprising a nucleotide sequence set forth in SEQ ID NO:1 or a variant thereof, or a complementary sequence thereto; an isolated antisense polynucleotide comprising at least 20 consecutive nucleotides complementary to a nucleotide sequence set forth in SEQ ID NO:1 or to a variant thereof; or an isolated polynucleotide comprising at least 15 consecutive nucleotides that is capable of hybridizing under moderately stringent conditions to a nucleotide sequence set forth in SEQ ID NO:1. In certain embodiments, the step of contacting a cell is performed in vitro.

According to certain other embodiments of the present invention there is provided a method of identifying an agent that is capable of altering PTP1B expression comprising: (a) contacting, in the absence and presence of a candidate agent, a sample comprising a Y-box protein and a recombinant nucleic acid construct that comprises a nucleotide sequence as set forth in SEQ ID NO:1 which is operably linked to a reporter gene, under conditions and for a time sufficient to detect transcription or expression of the reporter gene; and (b) comparing a level of reporter gene transcription or expression in the absence of the candidate agent to a level of reporter gene transcription or expression in the presence of the candidate agent, wherein a decreased level of reporter gene transcription or expression in the presence of the candidate agent relative to the level of reporter gene transcription or expression in the absence of the candidate agent indicates the agent is capable of altering PTP1B expression. In one embodiment the sample comprises a cell, and in another embodiment the sample comprises an isolated Y-box protein. In another embodiment the sample comprises an isolated recombinant nucleic acid construct. In certain embodiments the Y-box protein comprises an amino acid sequence that is SEQ ID NO:2 or SEQ ID NO:4. In another embodiment the reporter gene encodes a polypeptide that is luciferase or chloramphenicol acetyl transferase.

In certain other embodiments there is provided a method of identifying an agent that is capable of altering PTP1B expression comprising: (a) contacting a candidate agent and a biological sample comprising a cell that comprises a PTP1B gene and that is capable of PTP1B gene transcription or expression, under conditions and for a time sufficient to detect PTP1B gene transcription or expression; and (b) comparing a level of PTP1B gene transcription or expression in the absence of the candidate agent to a level of PTP1B gene transcription or expression in the presence of the candidate agent, wherein a decreased level of PTP1B gene transcription or expression in the presence of the candidate agent relative to the level of PTP1B gene transcription or expression in the absence of the candidate agent indicates the agent is capable of altering PTP1B expression. According to certain further embodiments the cell comprises an insulin receptor, the method further comprising determining a level of phosphorylation of the insulin receptor, wherein an increased level of insulin receptor phosphorylation in the presence of the candidate agent relative to the level of insulin receptor phosphorylation in the absence of the candidate agent indicates the agent is capable of altering PTP1B expression. In certain other further embodiments, the cell comprises an insulin receptor, the method further comprising determining a level of an insulin response in the cell, wherein an increased level of the insulin response in the presence of the candidate agent relative to the level of the insulin response in the absence of the candidate agent indicates the agent is capable of altering PTP1B expression. In certain still further embodiments the insulin response is glucose uptake, glycogen synthesis, lipogenesis, lipolysis, Glut4 recruitment to a plasma membrane or amino acid import. In certain other still further embodiments the insulin response is insulin receptor tyrosine phosphorylation, MAP kinase phosphorylation, AKT phosphorylation, inhibition of phosphoenolpyruvate carboxykinase transcription or phosphatidylinositoltriphosphate kinase activation. In certain other further embodiments the cell comprises a leptin receptor, the method further comprising determining a level of a leptin response in the cell, wherein an increased level of the leptin response in the presence of the candidate agent relative to the level of the leptin response in the absence of the candidate agent indicates the agent is capable of altering PTP1B expression. In certain still further embodiments, the leptin response is TYK2 phosphorylation, JAK2 phosphorylation, STAT1 phosphorylation or STAT3 phosphorylation.

These and other aspects of the present invention will become evident upon reference to the following detailed description and attached drawings. In addition, various references are set forth herein (and/or listed in the Application Data Sheet) which describe in more detail certain aspects of this invention, and are therefore incorporated by reference in their entireties.

### BRIEF DESCRIPTION OF THE DRAWINGS

FIG. 1 shows a schematic diagram of the human PTP1B promoter (FIG. 1A); a description of luciferase reporter constructs, which were used to define the enhancer sequence in the PTP1B promoter (FIG. 1B); and expression of the luciferase constructs in Rat1 cells (FIG. 1C).

FIG. 2 shows the results of an electrophoretic mobility shift assay (EMSA) using radiolabeled double-stranded oligonucleotide probes (FIG. 2A) to demonstrate protein/DNA complex formation with the transcription enhancing element for PTP1B expression (TEP) (FIG. 2B).

FIG. 3 shows the purification scheme (FIG. 3A) used to purify proteins that bind to the transcriptional enhancing element for PTP1B Expression (B-TEP). FIG. 3B shows the elution profile of nuclear extracts from a DNA-conjugated affinity column, and FIG. 3C presents the SDS-PAGE analysis of the eluate. The amino acid sequence of the SDS-PAGE purified B-TEP [SEQ ID NO:2] is shown in FIG. 3D. The double-underlined sequences are identical to portions of the amino acid sequence of rat Y box binding protein YB-1.

FIG. 4 shows immunoblot (IB) analyses of purified B-TEP (YB-1) and protein/DNA complexes. Purified YB-1 at various concentrations was immunobloted with an anti-YB-1 antibody (FIG. 4A). DNA probes were combined with purified B-TEP and the protein-DNA complexes were isolated by a DNA pull down assay procedure and then immunoblotted with an anti-YB-1 antibody (FIG. 4B). Nuclear extracts from Rat1 and HepG2 cells were incubated with biotinylated double-stranded DNA probe (−155/−132) and streptavidin-Sepharose, and the protein-DNA complexes were immunoblotted with anti-YB-1 antibody (upper panel of FIG. 4C). Total cell lysates were immunoblotted with antibodies specific for YB-1, PTP1B, or actin (lower panel of FIG. 4C).

FIG. 5 depicts the effect of YB-1 on PTP1B expression in Rat1 cells. Shown are immunoblot analyses of YB-1 and of the indicated protein tyrosine phosphatases from lysates of Rat1 cells that were stably transfected with YB-1 antisense constructs (FIG. 5A) and from protein-DNA complexes prepared from nuclear extracts of the transfected Rat1 cells (FIG. 5B). FIGS. 5C-D show the effect of depletion of cellular YB-1 on PTP1B promoter activity (FIG. 5C) and on PTP1B enhancer activity (FIG. 5D) using luciferase reporter constructs.

FIG. 6 shows insulin stimulation of Rat1 control cells and Rat1 cells stably transfected with a YB-1 antisense construct by immunoblot analysis of a cell lysate immunoprecipitated (IP) with an anti-insulin receptor β antibody, followed by immunoblotting with an anti-phosphotyrosine or an anti-insulin receptor β antibody (FIG. 6A). FIG. 6B shows an immunoblot analysis of a cell lysate using either an anti-phospho-Akt or an anti-Akt antibody as a probe. FIG. 6C presents an immunoblot analysis of a cell lysate using an anti-phospho-MAPK(Erk1/2) or an anti-MAPK(Erk1/2) antibody as a probe. The right-hand panels for FIGS. 6A-6C show for each indicated proband protein (IR-β, AKT, or MAPK), gel densitometry analyses of the ratio of phosphorylated protein to total protein, from Rat1 cells transfected with YB-1 antisense constructs (squares) and Rat1 control cells (circles). FIG. 6D shows insulin stimulation of Ratl cells that were stably transfected with YB-1-antisense RNA (Rat1-antisense YB-1) and then transiently transfected with a PTP1B expression vector. The left panel of FIG. 6D presents an immunoblot analysis of a cell lysate with an anti-PTP1B antibody (immunoblot a); immunoblot analysis of a cell lysate with an anti-actin antibody (immunoblot b); immunoblot analysis of a cell lysate immunoprecipitated with an anti-insulin receptor β antibody, followed by immunoblotting with an anti-phosphotyrosine antibody (immunoblot c); and immunoblot analysis of a cell lysate immunoprecipitated with an anti-insulin receptor β antibody, followed by immunoblotting with the same antibody (immunoblot d). The right panel of FIG. 6D represents densitometric analyses of the gel images to illustrate the ratio of phosphorylated protein to total protein in the absence (shaded bars) or presence (black bars) of ectopically expressed PTP1B.

FIG. 7 shows an immunoblot demonstrating the effect of gp130-mediated signaling in Rat1 control cells and Rat1 cells stably transfected with a YB-1 antisense construct, transiently transfected with a chimeric receptor construct comprising the extracellular domain of the G-CSF receptor and the transmembrane and cytoplasmic domains of gp130, and stimulated with G-CSF. FIG. 7A: immunoblot of a cell lysate immunoprecipitated with an anti-JAK1 antibody and immunoblotted with either an anti-phospho-JAK1 antibody or an anti-JAK1 antibody. FIG. 7B: immunoblot of a cell lysate immunoprecipitated with an anti-gp-130 antibody and immunoblotted with either an anti-phospho-tyrosine antibody or an anti-gp-130 antibody. FIG. 7C: immunoblot of a cell lysate using either an anti-phospho-STAT3 antibody or an anti-STAT3 antibody. FIG. 7D: immunoblot of a cell lysate using either an anti-phospho-MAPK(Erk1/2) or an anti-MAPK(Erk1/2) antibody. The right-hand panels show, for each indicated proband protein, gel densitometry analyses of the ratio of phosphorylated protein to total protein, from Rat1 cells transfected with YB-1 antisense constructs (squares) and Rat1 control cells (circles). FIG. 7E shows G-CSF stimulation of Rat1 cells that were stably transfected with YB-1-antisense RNA (Rat1-antisense YB-1) and then transiently co-transfected with a PTP1B expression vector (or empty vector control) and a G-CSFR-gp130 recombinant expression vector. The left panel of FIG. 7E presents an immunoblot analysis of a cell lysate using an anti-PTP1B antibody (immunoblot a); immunoblot analysis of a cell lysate using an anti-actin antibody (immunoblot b); immunoblot analysis of a cell lysate immunoprecipitated with an anti-JAK1 antibody, followed by immunoblotting with an anti-phospho-JAK1 antibody (immunoblot c); and immunoblot analysis of a cell lysate immunoprecipitated with an anti-JAK1 antibody, followed by immunoblotting with the same antibody (immunoblot d). The right panel of FIG. 7E represents densitometric analyses of the gel images to illustrate the ratio of phosphorylated protein to total protein in the absence (shaded bars) or presence (black bars) of ectopically expressed PTP1B.

FIG. 8 shows a schematic representation of a proposed mechanism by which regulation of PTP1B expression affects the phosphorylation state of the insulin receptor (IR).

FIG. 9 illustrates the results of an EMSA detecting the interaction of purified YB-1 with PTP1B enhancer probes. FIG. 9A, lane 1: YB-1:³²P-labeled DNA −155/−132 probe complex (YB-1: DNA complex) in the absence of antibody; lane 2: YB-1 DNA complex plus anti-YB-1 antibody; lane 3: YB-1:DNA complex plus an anti-GATA1 antibody; lane 4: YB-1:DNA complex plus an anti-GATA2 antibody. FIG. 9B illustrates the interaction of a recombinant GST-YB-1 fusion protein with a PTP1B enhancer probe, −167/−132. GST and recombinant GST-YB-1 fusion protein were expressed, purified, and analyzed by SDS-PAGE (see FIG. 9B, left panel). Aliquots of purified GST and GST-YB-1 (1 and 10 ng) were combined with a ³²P-labeled DNA (−167/−132) probe and analyzed by EMSA as shown in FIG. 9B, right panel. Lane 1: 32P(−167/−132) probe only; lane 2: 1 ng GST plus 32P(−167/−132) probe; lane 3: 10 ng GST plus 32P-(167/−132) probe; lane 4: 1 ng GST-YB-1 plus 32P(−167/−132) probe; lane 5: 10 ng GST-YB-1 plus 32P(−167/−132) probe; lane 6: 10 ng GST-YB-1 plus ³²P(−167/−132) probe plus unlabeled (167/−132); lane 7: 10 ng GST-YB-1 plus ³²P(−167/−132) probe plus unlabeled −167/−132 (Δ−155/−144) competitor DNA.

FIG. 10 illustrates overexpression of YB-1 in Rat1 cells. Rat1 cells were transiently transfected with 10 μg of expression plasmid containing either FLAG®-tagged YB-1 (Flag-YB-1) or pCMV-tag empty vector control (control). Total cell lysates were analyzed by immunoblot using anti-YB-1, anti-PTP1B, or anti-actin antibodies (FIG. 10A). Densitometric analyses of the gel images are presented in FIG. 10B, in which expression of YB-1 (black bars) and PTP1B (shaded bars) is presented relative to the actin control.

FIG. 11 shows a densitometric analysis of an agarose gel (inset) of YB-1 cDNA and PTP1B cDNA amplified by PCR from a Human Tumor Multiple Tissue cDNA (MTC™) Panel (BD Biosciences Clontech) (FIG. 11A). FIG. 11B illustrates an immunoblot (left panel) of YB-1 and PTP1B polypeptides expressed from two non-obese, insulin-resistant type II diabetic Goto-Kakazaki rats (animals designated D1 and D2) and from two non-diabetic, control WKY rats (animals designated C1 and C2). Skeletal muscle homogenates from each animal were separated by SDS-PAGE and then immunoblotted with an anti-YB-1 antibody, an anti-PTP1B antibody, or an anti-actin antibody (FIG. 11B, left panel). FIG. 11B, right panel, represents densitometric analyses of the immunoblot to illustrate the ratio of expression of YB-1 and PTP1B compared with the actin control. The black bars represent the expression ratio of YB-1 to actin and the shaded bars represent the expression ratio of PTP1B to actin.

### DETAILED DESCRIPTION OF THE INVENTION

The present invention is directed in part to the unexpected discovery of a Y-box protein binding site that is present as a transcription enhancer sequence in the promoter region situated upstream (i.e., 5′ to) of the PTP1B gene. This site, situated at nucleotides −155 through −132 of the human PTP1B gene according to a nucleotide numbering scheme whereby nucleotide −1 is located immediately 5′ to the PTP1B translation start site (Fukada et al., 2001 J. Biol. Chem. 276:25512), is defined according to the present invention as

5′-AGATATCTCGCGGTGCTGGGGCCA-3′ [SEQ ID NO:1]

and surprisingly, as provided herein, is shown for the first time to comprise a binding recognition element for the YB-1 transcription factor, a member of the Y-box family of DNA binding proteins. Y-box proteins comprise a highly conserved family of transcriptional and translational regulators of gene expression (Swamynathan et al., 1998 FASEB J. 12:515; Matsumoto et al., 1998 Trends Cell Biol. 8:318; Duh et al., 1995 J. Biol. Chem. 270:30499; Didier et al., 1988 Proc. Nat. Acad. Sci. USA 85:7322; MacDonald et al. 1995 J. Biol. Chem. 270:3527; Koike et al., 1997 FEBS Lett. 417:390; Bargou et al., 1997 Nat. Med. 3:447; Ohga et al., 1998 J. Biol. Chem. 273:5997; Spitkovsky et al., 1992 Nucleic Acids Res. 20:797-803; Norman et al., 2001 J. Biol. Chem. 276:29880; Wolffe, Bioessay 16:245-51 (1994); Grant et al., Mol. Cell Biol. 13:4186-96 (1993)). (See also, e.g., Dorn et al., Cell 50:836-72 (1987); Dorn et al., Proc. Natl. Acad. Sci. USA 84:6249-53 (1987); Mertens et al. J. Biol. Chem. 273:32957-65 (1998); Okamoto et al., Oncogene 19:6194-6202 (2000); Lasham et al., Gene 252:1-13 (2000)). As described in greater detail herein, the observation that PTP1B expression can be regulated by YB-1 may be exploited according to certain embodiments of the present invention to treat a PTP1B associated disorder, for example, diabetes, obesity or other metabolic disorders. The invention thus provides useful compositions and methods for intervention in the regulation of biological signal transduction pathways that comprise PTP1B, and for the first time offers an alternative to PTP active site-directed strategies for modulation of PTP1B activity.

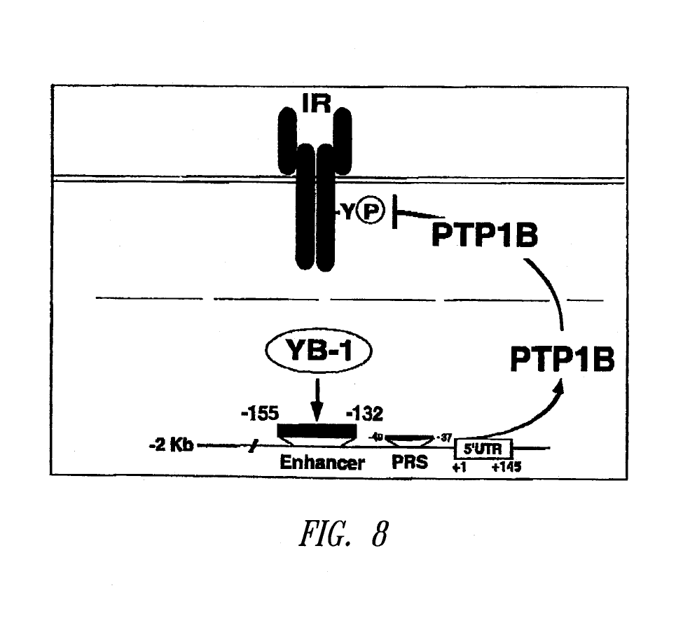

Accordingly, in certain embodiments the present invention provides an agent for treating a PTP1B associated disorder, which agent impairs binding of a Y-box protein (e.g., YB-1) to a PTP1B promoter Y-box protein binding site (e.g., SEQ ID NO:1) in a cell, and in certain embodiments the invention provides a method for identifying such an agent that is capable of altering (i.e., increasing or decreasing in a statistically significant manner relative to an appropriate control) PTP1B expression. Without wishing to be bound by theory, according to the present invention there is provided in the PTP1B gene promoter region an upstream enhancer sequence (SEQ ID NO:1) that functions as a YB-1 recognition element such that interference with YB-1 binding to this enhancer, for instance, by antisense inhibition of YB-1 expression, diminishes PTP1B expression and leads to hyperphosphorylation of PTP1B substrates (e.g., insulin receptor (IR), FIG. 8). As a result, and further according to non-limiting theory, downstream effects of impaired YB-1 binding to the PTP1B promoter Y-box protein binding site are manifest in the PTP1B biological signal transduction pathway, for example, enhancement of insulin signaling sequelae such as activation (e.g., by phosphorylation) of kinase substrate proteins (e.g., signaling pathway components such as AKT or MAP kinase).

Thus, it is an aspect of the present invention to provide an isolated polynucleotide comprising the nucleotide sequence set forth in SEQ ID NO:1, or a complementary sequence thereto, which sequence is present at nucleotides −155 through −132 of the human PTP1B gene and which sequence is shown, as described in greater detail below, to act as a YB-1 transcriptional enhancer within the human PTP1B gene promoter region.

A “PTP1B −155/−132 polynucleotide” is any polynucleotide that comprises the nucleic acid sequence set forth in SEQ ID NO:1 or a variant thereof, or that is complementary to such a polynucleotide, wherein the PTP1B −155/−132 polynucleotide is capable of detectably binding to a human YB-1 polypeptide (SEQ ID NO:4). Preferred polynucleotides comprise at least 15, 16, 17, 18 or 19 consecutive nucleotides, preferably at least 20, 21, 22, 23 or 24 consecutive nucleotides, of the PTP1B −155/−132 polynucleotide sequence set forth in SEQ ID NO:1, or a polynucleotide complementary to such a sequence. By way of example, a polynucleotide comprising at least 20 consecutive nucleotides of the PTP1B −155/−132 polynucleotide sequence may have one or more nucleotides less at either the 5′ or the 3′ end (e.g., AGATATCTCGCGGTGCTGGG [SEQ ID NO:43]; AGATATCTCGCGGTGCTGGGG [SEQ ID NO:44]; AGATATCTCG CGGTGCTGGGGC [SEQ ID NO:45]; AGATATCTCG CGGTGCTGGGGCC [SEQ ID NO:46]; ATCTCG CGGTGCTGGGGCCA [SEQ ID NO:47]; TATCTCG CGGTGCTGGGGCCA [SEQ ID NO:48]; ATATCTCG CGGTGCTGGGGCCA [SEQ ID NO:49]; GATATCTCG CGGTGCTGGGGCCA [SEQ ID NO:50]). Alternatively, a PTP1B −155/−132 polynucleotide sequence may comprise a sequence of at least 15, 16, 17, 18, 19, or 20 consecutive nucleotides of the sequence set forth in SEQ ID NO:1, but which has at least one nucleotide substitution (e.g., AGTTATCTCGCGGTGCTGGGGCCA [SEQ ID NO:40]; AGATATCTCGCGGTGCTGGCGCCA [SEQ ID NO:41]; or AGATATCT CGCGGAG CTGGGCCCA [SEQ ID NO:42]), or the like.

Certain polynucleotides may find use as probes, primers or antisense oligonucleotides, as described below. Polynucleotides may be single-stranded (coding or antisense) or double-stranded, and may be DNA (genomic, cDNA or synthetic) or RNA molecules. Additional coding or non-coding sequences may, but need not, be present within a polynucleotide of the present invention, and a polynucleotide may, but need not, be linked to other molecules and/or support materials.

PTP1B −155/−132 polynucleotides may comprise a native sequence (i.e., an endogenous PTP1B −155/−132 sequence, or a portion thereof) or may comprise a variant of such a sequence. Polynucleotide variants may contain one or more substitutions, additions, deletions and/or insertions such that the YB-1 binding activity to the polynucleotide is not substantially diminished. The effect on the activity of the polynucleotide may generally be assessed as described herein and/or according to procedures provided by the cited references. Variants preferably exhibit at least about 70% identity, more preferably at least about 80% identity, more preferably at least about 83%, 84%, or 85% identity, and most preferably at least about 90% identity to the polynucleotide sequence of SEQ ID NO:1. A variant of SEQ ID NO:1 may include a nucleotide sequence in which at least one nucleotide base has been substituted with another nucleotide base. For example, as discussed herein, variants of SEQ ID NO:1 [AGATATCTCG CGGTGCTGGGGCCA] may include a polynucleotide comprising the sequence AGTTATCTCGCGGTGCTGGGGCCA [SEQ ID NO:40], AGATATCTCG CGGTGCTGGCGCCA [SEQ ID NO:41], or AGATATCTCGCGGAGCTGGGCCCA [SEQ ID NO:42], or other variants of SEQ ID NO:1 that retain the capability of specifically hybridizing to a target DNA or RNA sequence as described herein. The percent identity may be readily determined by comparing sequences using computer algorithms well known to those having ordinary skill in the art, such as Align or the BLAST algorithm (Altschul, J. Mol. Biol. 219:555-565, 1991; Henikoff and Henikoff, Proc. Natl. Acad. Sci. USA 89:10915-10919, 1992), which is available at the NCBI website (http://www/ncbi.nlm.nih.gov/cgi-bin/BLAST). Default parameters may be used. Certain variants are substantially homologous to a native gene sequence. Such polynucleotide variants are capable of hybridizing under moderately stringent conditions to a naturally occurring DNA or RNA sequence corresponding to a native PTP1B −155/−132 polynucleotide sequence (e.g., SEQ ID NO:1), or a complementary sequence thereto. Suitable moderately stringent conditions include, for example, prewashing in a solution of 5×SSC, 0.5% SDS, 1.0 mM EDTA (pH 8.0); hybridizing at 50° C.-70° C., 5×SSC, for 1-16 hours (e.g., overnight); followed by washing once or twice at 22-65° C. for 20-40 minutes with one or more each of 2×, 0.5× and 0.2× SSC containing 0.05-0.1% SDS. For additional stringency, conditions may include a wash in 0.1× SSC and 0.1% SDS at 50-60° C. for 15-40 minutes. As known to those having ordinary skill in the art, variations in stringency of hybridization conditions may be achieved by altering the time, temperature and/or concentration of the solutions used for prehybridization, hybridization and wash steps, and suitable conditions may also depend in part on the particular nucleotide sequences of the probe used, and of the blotted, proband nucleic acid sample. Accordingly, it will be appreciated that suitably stringent conditions can be readily selected without undue experimentation where a desired selectivity of the probe is identified, based on its ability to hybridize to one or more certain proband sequences while not hybridizing to certain other proband sequences.

Polynucleotides may be prepared using any of a variety of techniques. For example, a polynucleotide may be amplified from genomic DNA prepared from a suitable cell or tissue type, such as a cell line or human skeletal muscle cells. Such polynucleotides may be amplified via polymerase chain reaction (PCR). For this approach, sequence-specific primers may be designed based on the sequences provided herein, and may be purchased or synthesized.

An amplified portion may be used to isolate all or a portion of a full length PTP1B gene (i.e., including regulatory sequences that include nucleotides −155/−132 upstream from the transcription start site) from a suitable library (e.g., a human cell line or tissue-derived DNA library such as a genomic DNA library) using well known techniques (e.g., Fukada et al., 2001 J. Biol. Chem. 276:25512). Within such techniques, a DNA library is screened using one or more polynucleotide probes or primers suitable for amplification. Preferably, a library is size-selected to include larger molecules. Random primed libraries may also be preferred for identifying 5′ and upstream regions of genes. Genomic libraries are preferred for obtaining introns and extending 5′ sequences.

For hybridization techniques, a partial sequence may be labeled (e.g., by nick-translation or end-labeling with ³²P) using well known techniques. A bacterial or bacteriophage library may then be screened by hybridizing filters containing denatured bacterial colonies (or lawns containing phage plaques) with the labeled probe (see, e.g., Sambrook et al., Molecular Cloning: A Laboratory Manual, Cold Spring Harbor Laboratory Press, Cold Spring Harbor, N.Y., 2001). Hybridizing colonies or plaques are selected and expanded, and the DNA is isolated for further analysis. Clones may be analyzed to determine the amount of additional sequence by, for example, PCR using a primer from the partial sequence and a primer from the vector. Restriction maps and partial sequences may be generated to identify one or more overlapping clones. A full length DNA or cDNA molecule of interest can be generated by ligating suitable fragments, using well known techniques.

Alternatively, there are numerous amplification techniques for obtaining an extended sequence from a partial DNA or cDNA sequence. Within such techniques, amplification is generally performed via PCR. One such technique for generating a full length coding sequence from a partial cDNA sequence is known as “rapid amplification of cDNA ends” or RACE; all or a portion of a full length coding sequence so obtained may be used to obtain upstream gene regulatory sequences from, for instance, a genomic DNA library. These techniques involve the use of an internal primer and an external primer, which hybridizes to a polyA region or vector sequence, to identify sequences that are 5′ and 3′ of a known sequence. Any of a variety of commercially available kits may be used to perform the amplification step. Primers may be designed using, for example, software well known in the art. Primers are preferably 17-32 nucleotides in length, have a GC content of at least 40% and anneal to the target sequence at temperatures of about 54° C. to 72° C. The amplified region may be sequenced as described above, and overlapping sequences assembled into a contiguous sequence.

A DNA sequence for PTP1B −155/−132 is provided in SEQ ID NO:1. PTP1B −155/−132 polynucleotide variants may generally be prepared by any method known in the art, including, for example, solid phase chemical synthesis. Modifications in a polynucleotide sequence may also be introduced using standard mutagenesis techniques, such as oligonucleotide-directed site-specific mutagenesis. Alternatively, RNA molecules may be generated by in vitro or in vivo transcription of DNA sequences for PTP1B −155/−132, or a portion thereof, provided that the DNA is incorporated into a vector with a suitable RNA polymerase promoter (such as T7 or SP6).

In addition, or alternatively, certain embodiments of the invention contemplate use of a polynucleotide as provided herein that is a YB-1 antisense polynucleotide, for example, as described below and by Ohga et al. (1997 J. Biol. Chem. 273:5997), or a YB-1 encoding polynucleotide (e.g., Ohga et al., 1997; Didier et al., 1988 Proc. Nat. Acad. Sci. USA 85:7322; MacDonald et al. 1995 J. Biol. Chem. 270:3527; Norman et al., 2001 J. Biol. Chem. 276:29880; and references cited therein; U.S. Pat. No. 6,140,126). Such a polynucleotide may be administered to a patient such that the YB-1 antisense polynucleotide (or encoded YB-1 polypeptide) is generated in vivo, and in the case of an encoded polypeptide, it will also be appreciated by those having ordinary skill in the art that, as a result of the degeneracy of the genetic code, there are many nucleotide sequences that can encode a particular polypeptide. Some of these polynucleotides bear minimal homology to the nucleotide sequence of any native gene. Nonetheless, polynucleotides that vary due to differences in codon usage are specifically contemplated by the present invention.

An antisense molecule as contemplated by the invention may also include a polynucleotide that is complementary to a non-coding portion of a gene. In certain embodiments of the invention, a YB-1 antisense polynucleotide may be complementary to a non-coding portion of the YB-1 gene, for example, to a regulatory sequence that is 5′ (upstream) of the coding portion (see U.S. Pat. No. 6,140,126). In another embodiment, the invention contemplates use of an antisense polynucleotide that is complementary to the PTP1B −155/−132 sequence (e.g., an antisense polynucleotide complementary to SEQ ID NO:1). Persons skilled in the art understand that the sequence of an antisense compound need not be 100% complementary to that of its target nucleic acid to be specifically hybridizable. Hybridization refers to hydrogen bonding, which may be Watson-Crick, Hoogsteen or reversed Hoogsteen hydrogen bonding, between complementary nucleoside or nucleotide bases. The term complementary as used herein refers to the capability for precise pairing between two nucleotides. An antisense polynucleotide that is specifically hybridizable (or complementary) is a polynucleotide that has a sufficient degree of complementarity to a DNA or RNA target sequence to stably and specifically bind to the DNA or RNA target. Such binding interferes with the normal function of the target DNA or RNA. A polynucleotide that has a sufficient degree of complementarity to a DNA or RNA target sequence also forestalls binding of the antisense polynucleotide to a non-target sequence under physiological conditions (in vivo) or under appropriate assay conditions (in vitro).

A polynucleotide that is complementary to at least a portion of a coding sequence (e.g., an antisense polynucleotide or a ribozyme, such as a YB-1-specific antisense polynucleotide) may also be used as a probe or primer, or to modulate gene expression. Identification of oligonucleotides and ribozymes for use as antisense agents, and DNA encoding genes for their targeted delivery, involve methods well known in the art. For example, the desirable properties, lengths and other characteristics of such oligonucleotides are well known. Antisense oligonucleotides are typically designed to resist degradation by endogenous nucleolytic enzymes by using such linkages as: phosphorothioate, methylphosphonate, sulfone, sulfate, ketyl, phosphorodithioate, phosphoramidate, phosphate esters, and other such linkages (see, e.g., Agrwal et al., Tetrahedron Lett. 28:3539-3542 (1987); Miller et al., J. Am. Chem. Soc. 93:6657-6665 (1971); Stec et al., Tetrahedron Lett. 26:2191-2194 (1985); Moody et al., Nucleic Acids Res. 12:4769-4782 (1989); Uznanski et al., Nucleic Acids Res. (1989); Letsinger et al., Tetrahedron 40:137-143 (1984); Eckstein, Annu. Rev. Biochem. 54:367-402 (1985); Eckstein, Trends Biol. Sci. 14:97-100 (1989); Stein In: Oligodeoxynucleotides. Antisense Inhibitors of Gene Expression, Cohen, Ed, Macmillan Press, London, pp. 97-117 (1989); Jager et al., Biochemistry 27:7237-7246 (1988)).

Antisense polynucleotides are oligonucleotides that bind in a sequence-specific manner to nucleic acids, such as mRNA or DNA. When bound to mRNA that has complementary sequences, antisense prevents translation of the mRNA (see, e.g., U.S. Pat. No. 5,168,053 to Altman et al.; U.S. Pat. No. 5,190,931 to Inouye, U.S. Pat. No. 5,135,917 to Burch; U.S. Pat. No. 5,087,617 to Smith and Clusel et al. (1993) Nucleic. Acids Res. 21:3405-3411, which describes dumbbell antisense oligonucleotides). Triplex molecules refer to single DNA strands that bind duplex DNA forming a colinear triplex molecule, thereby preventing transcription (see, e.g., U.S. Pat. No. 5,176,996 to Hogan et al., which describes methods for making synthetic oligonucleotides that bind to target sites on duplex DNA).

Particularly useful antisense nucleotides and triplex molecules are molecules that are complementary to or bind the sense strand of DNA or mRNA that encodes a YB-1 polypeptide or a protein mediating any other process related to expression of endogenous PTP1B that relates to the PTP1B −155/−132 upstream regulatory element, such that inhibition of transcription and/or translation of mRNA encoding the PTP1B polypeptide is effected. cDNA constructs that can be transcribed into antisense RNA may also be introduced into cells or tissues to facilitate the production of antisense RNA. Antisense technology can be used to control gene expression through interference with binding of polymerases, transcription factors or other regulatory molecules (see Gee et al., In Huber and Carr, Molecular and Immunologic Approaches, Futura Publishing Co. (Mt. Kisco, N.Y.; 1994)). Alternatively, an antisense molecule may be designed to hybridize with a control region of a PTP1B gene (e.g., a PTP1B promoter, a PTP1B −155/−132 enhancer or a PTP1B transcription initiation site), and block transcription of the gene; or to block translation by inhibiting binding of a transcript to ribosomes.

The present invention also contemplates the use of ribozymes, such as YB-1 specific ribozymes. A ribozyme is an RNA molecule that specifically cleaves RNA substrates, such as mRNA, resulting in specific inhibition or interference with cellular gene expression. There are at least five known classes of ribozymes involved in the cleavage and/or ligation of RNA chains. Ribozymes can be targeted to any RNA transcript and can catalytically cleave such transcripts (see, e.g., U.S. Pat. No. 5,272,262; U.S. Pat. No. 5,144,019; and U.S. Pat. Nos. 5,168,053, 5,180,818, 5,116,742 and 5,093,246 to Cech et al.). Any mRNA-specific ribozyme, or a nucleic acid encoding such a ribozyme, may be delivered to a host cell to effect specific inhibition of gene expression. Ribozymes may therefore be delivered to the host cells by DNA encoding the ribozyme linked to a eukaryotic promoter, such as a eukaryotic viral promoter, such that upon introduction into the nucleus, the ribozyme will be directly transcribed.

Any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine and wybutosine, as well as acetyl- methyl-, thio- and other modified forms of adenine, cytidine, guanine, thymine and uridine.

Nucleotide sequences as described herein may be joined to a variety of other nucleotide sequences using established recombinant DNA techniques, to produce recombinant nucleic acid constructs. For example, a polynucleotide may be cloned into any of a variety of cloning vectors, including plasmids, phagemids, lambda phage derivatives and cosmids. Recombinant nucleic acid constructs of particular interest include vectors such as expression vectors, replication vectors, probe generation vectors and sequencing vectors. In general, a suitable vector contains an origin of replication functional in at least one organism, convenient restriction endonuclease sites and one or more selectable markers. Most preferably two or more polynucleotide sequence regions in a recombinant nucleic acid construct as provided herein are positioned relative to one another in operable linkage, such that desired structures and/or functions (e.g., promoter-driven transcription, translatable transcripts, fusion sequences, etc.) may be obtained. Other elements will depend upon the desired use, and will be apparent to those having ordinary skill in the art.

Within certain embodiments, polynucleotides may be formulated so as to permit entry into a cell of a mammal, and expression therein. Such formulations are particularly useful for therapeutic purposes, as described below. Those having ordinary skill in the art will appreciate that there are many ways to achieve expression of a polynucleotide in a target cell, and any suitable method may be employed. For example, a polynucleotide may be incorporated into a viral vector using well known techniques. A viral vector may additionally transfer or incorporate a gene for a selectable marker (to aid in the identification or selection of transduced cells) and/or a targeting moiety, such as a gene that encodes a ligand for a receptor on a specific target cell, to render the vector target specific. Targeting may also be accomplished using an antibody, by methods known to those having ordinary skill in the art.

Other formulations for therapeutic purposes include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Within other aspects, all or a portion of a PTP1B gene promoter region or segment that includes the PTP1B −155/−132 polynucleotide sequence may be isolated using standard techniques. The present invention provides nucleic acid molecules comprising such a promoter sequence or one or more cis- or trans-acting regulatory elements thereof. Such regulatory elements may enhance or suppress expression of PTP1B. A 5′ flanking region may be generated using standard techniques, based on the partial genomic sequence provided herein or using the PTP1B polypeptide coding sequence known to the art. If necessary, additional 5′ sequences may be generated using PCR-based or other standard methods. The 5′ region may be subcloned and sequenced using standard methods. Primer extension and/or RNase protection analyses may be used to verify the transcriptional start site deduced from the cDNA.

To define the boundary of the promoter region, putative promoter inserts of varying sizes may be subcloned into a heterologous expression system containing a suitable reporter gene without a promoter or enhancer. Suitable reporter genes may include genes encoding luciferase, beta-galactosidase, chloramphenicol acetyl transferase, secreted alkaline phosphatase or the Green Fluorescent Protein gene. Suitable expression systems are well known and may be prepared using well known techniques or obtained commercially. Internal deletion constructs may be generated using unique internal restriction sites or by partial digestion of non-unique restriction sites. Constructs may then be transfected into cells that display or that are capable of high levels of PTP1B expression. In general, the construct with the minimal 5′ flanking region showing the highest level of expression of reporter gene is identified as the promoter. Such promoter regions, and as described in greater detail below, preferably a PTP1B −155/−132 polynucleotide as provided herein, may be linked to a reporter gene and used to evaluate agents for the ability to alter (e.g., increase or decrease in a statistically significant manner) or modulate PTP1B expression, for example by altering transcription and/or translation of PTP1B mRNA.

Once a functional promoter is identified, cis- and trans-acting elements may be located. Cis-acting sequences may generally be identified based on homology to previously characterized transcriptional motifs. Point mutations may then be generated within the identified sequences to evaluate the regulatory role of such sequences. Such mutations may be generated using site-specific mutagenesis techniques or a PCR-based strategy. The altered promoter is then cloned into a reporter gene expression vector, as described above, and the effect of the mutation on reporter gene expression is evaluated. The present invention also contemplates the use of allelic variants of PTP1B −155/−132 polynucleotides, as well as sequences from other organisms that correspond to PTP1B −155/−132 polynucleotides. Such sequences may generally be identified based upon similarity to the sequences provided herein (e.g., using hybridization techniques) and based upon the presence of PTP1B −155/−132 YB-1 binding activity, using an assay provided herein.

In general, polypeptides and polynucleotides as described herein are isolated. An “isolated” polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a recombinant nucleic acid construct such as a vector that is not a part of the natural environment. A “gene” includes the segment of DNA involved in producing a polypeptide chain; it further includes regions preceding and following the coding region “leader and trailer”, for example promoter and/or enhancer and/or other regulatory sequences and the like, as well as intervening sequences (introns) between individual coding segments (exons).

As noted above, according to certain embodiments of the invention there are provided compositions and methods that relate to altering or altered PTP1B expression, and/or to a PTP1B associated disorder. A PTP1B associated disorder includes any disease, disorder, condition, syndrome, pathologic or physiologic state, or the like, wherein at least one undesirable deviation or departure from a physiological norm causes, correlates with, is accompanied by or results from an inappropriate alteration (i.e., a statistically significant change) to the structure, activity, function, expression level, physicochemical or hydrodynamic property, or stability of a PTP1B or of a molecular component of a biological signal transduction pathway that comprises a PTP1B. In preferred embodiments the molecular component may be a protein, peptide or polypeptide, and in certain other preferred embodiments the alteration may be an altered level of PTP1B expression. In certain other preferred embodiments the alteration may be manifest as an a typical or unusual phosphorylation state of a protein under particular conditions, for example, hypophosphorylation or hyperphosphorylation of a phosphoprotein, wherein those familiar with the art will appreciate that phosphorylated proteins typically comprise one or more phosphotyrosine, phosphoserine or phosphothreonine residues.

PTP1B associated disorders therefore include diabetes mellitus, obesity, impaired glucose tolerance and other metabolic disorders wherein alteration of PTP1B or of a PTP1B signaling pathway component is associated with the disorder, but the invention is not intended to be so limited and contemplates other disorders, such as cancer, autoimmunity, cellular proliferative disorders and infectious disease (see e.g., Fukada et al., 2001 J. Biol. Chem. 276:25512; Tonks et al., 2001 Curr. Opin. Cell Biol. 13:182; Salmeen et al.,2000 Mol. Cell 6:1401; and references cited therein). Persons skilled in the art will be familiar with an array of criteria according to which it may be recognized what are, e.g., biological, physiological, pathological and/or clinical signs and/or symptoms of PTP1B disorders as provided herein.

As noted above, regulated tyrosine phosphorylation contributes to specific pathways for biological signal transduction, including those associated with cell division, cell survival, apoptosis, proliferation and differentiation, and “biological signal transduction pathways” or “inducible signaling pathways” in the context of the present invention include transient or stable associations or interactions among molecular components involved in the control of these and similar processes in cells. Depending on the particular pathway of interest, an appropriate parameter for determining induction of such pathway may be selected. For example, for signaling pathways associated with cell proliferation, there is available a variety of well known methodologies for quantifying proliferation, including, for example, incorporation of tritiated thymidine into cellular DNA, monitoring of detectable (e.g., fluorimetric or calorimetric) indicators of cellular respiratory activity, or cell counting, or the like. Similarly, in the cell biology arts there are known multiple techniques for assessing cell survival (e.g., vital dyes, metabolic indicators, etc.) and for determining apoptosis (e.g., annexin V binding, DNA fragmentation assays, caspase activation, etc.). Other signaling pathways will be associated with particular cellular phenotypes, for example specific induction of gene expression (e.g., detectable as transcription or translation products, or by bioassays of such products, or as nuclear localization of cytoplasmic factors), altered (e.g., statistically significant increases or decreases) levels of intracellular mediators (e.g., activated kinases or phosphatases, altered levels of cyclic nucleotides or of physiologically active ionic species, etc.), or altered cellular morphology, and the like, such that cellular responsiveness to a particular stimulus as provided herein can be readily identified to determine whether a particular cell comprises an inducible signaling pathway.

Additional useful information can be found, for example, in Diamond et al., J. Biol. Chem. 276:7943-51 (2001); Pearson et al., Mol. Cell Biol. 11:2081-95(1991); Sabath et al., J. Biol. Chem. 265:12671-78 (1990); Sakura, et al., Gene 73:499-507 (1988); Coles et al., Nucleic Acids Res. 24:2311-17 (1996); Fukada et al., Growth Factors 17:81-91 (1999); Gaikwad et al., Gene 204:79-83 (1997); Ito et al., Nucleic Acids Res. 22:2036-41 (1994); Kolluri et al., Nucleic Acids Res. 20:111-16 (1992); Sreenath et al., Cancer Res. 52:4942-47 (1992); Stetler-Stevenson et al., Annu. Rev. Cell Biol. 9:541-73 (1993); Sundseth et al., Mol. Pharmacol. 51:963-71 (1997); Travali et al., J. Biol. Chem. 264:7466-72 (1989).

Certain embodiments of the present invention are directed to compositions and methods that relate to a Y-box protein, which includes any member of a highly conserved family of proteins that are capable of interacting with DNA and RNA and that possess a conserved domain of approximately 70 amino acids termed the cold-shock domain, which is capable of binding to a DNA “Y-box” sequence (ATTGG) as described by Matsumoto et al. (1998 Trends Cell Biol. 8:318) and Swamynathan et al. (1998 FASEB. J. 12:515). In certain particularly preferred embodiments, and as described in greater detail below, the Y-box protein may be a human, rat or mouse YB-1 (e.g., Duh et al., 1995 J. Biol. Chem. 270:30499; Didier et al., 1988 Proc. Nat. Acad. Sci. USA 85:7322; MacDonald et al. 1995 J. Biol. Chem. 270:3527; Koike et al., 1997 FEBS Lett. 417:390; Bargou et al., 1997 Nat. Med. 3:447; Ohga et al., 1998 J. Biol. Chem. 273:5997; Spitkovsky et al., 1992 Nucleic Acids Res. 20:797-803; Norman et al., 2001 J. Biol. Chem. 276:29880) or a variant or fragment thereof, or an analog, homolog, ortholog or other derivative, or the like, including such Y-box proteins as may be identified on the basis of sequence similarity and that are capable of binding to a PTP1B promoter Y-box protein binding site, such as that defined by PTP1B −155/−132.

Representative examples of YB-1 polynucleotide and/or polypeptide sequences may be found, by way of illustration and not limitation, at the following GENBANK accession numbers: (human YB-1) J03827 (SEQ ID NO:4); AAA35750.1 (SEQ ID NO:54); (rat YB-1) NP_{—}113751 (SEQ ID NO:51); M57299 (SEQ ID NO:52 and SEQ ID NO:53); AAB46889.2 (SEQ ID NO:58); BAA02569.1 (SEQ ID NO:57); (mouse YB-1) AAA75476.1 (SEQ ID NO:55); AAA63390.1 (SEQ ID NO:56). It should be noted that additional Y-box proteins and encoding nucleotide sequences for use according to the instant invention may be selected, using sequence alignment tools and databases as known in the art and as provided herein, and further, as provided herein, using assays disclosed herein for determining binding of such proteins to a PTP1B promoter Y-box protein binding site, such as that defined by PTP1B −155/−132. For instance, identification of additional YB-1 proteins may be achieved by comparing sequences using computer algorithms well known to those having ordinary skill in the art, such as GENEWORKS, Align or the BLAST algorithm (Altschul, J. Mol. Biol. 219:555-565, 1991; Henikoff and Henikoff, Proc. Natl. Acad. Sci. USA 89:10915-10919, 1992), which is available at the NCBI website (http://www/ncbi.nlm.nih.gov/cgi-bin/BLAST).

For example, a representative YB-1 amino acid sequence is as follows [SEQ ID NO:4]:

| | |
|---|---|
| MSSEAETQQPPAAPPAAPALSAADTKPGTTGSGAGSGGPGGLTSAAPA | [SEQ ID NO:4] |
| GGDKKVIATKVLGTVKWFNVRNGYGFINRNDTKEDVFVHQTAIKKNNPRKYLRSVG | |
| DGETVEFDVVEGEKGEEAANVTGPGGVPVQGSKYAADRNHYRRYRRRGPPRNYQQ | |
| NYQNSESGEKNEGSESAPEGQAQQRRPYRRRRFPPYYMRRPYGRRPQYSNPPVQGEV | |
| MEGADNQGAGEQGRPVRQNMYRGYRPRFRRGPPRQRQPREDGNEEDKENQGDETQ | |
| GQQPPQRRYRRNFNYRRRRPENPKPQDGKETKAADPPAENSRSRG | |

The YB-1 polypeptide of SEQ ID NO:4 is encoded by the following polynucleotide sequence [SEQ ID NO:3]:

| | | |
|---|---|---|
| | 5′ --- | [SEQ ID NO:3] |
| 1 | ccgggagcgg agagcggacc ccagagagcc ctgagcagcc ccaccgccgc cgccggccta | |
| 61 | gttaccatca caccccggga ggagccgcag ctgccgcagc cggccccagt caccatcacc | |
| 121 | gcaaccatga gcagcgaggc cgagacccag cagccgcccg ccgccccccc cgccgccccc | |
| 181 | gccctcagcg ccgccgacac caagcccggc actacgggca gcggcgcagg gagcggtggc | |
| 241 | ccgggcggcc tcacatcggc ggcgcctgcc ggcggggaca agaaggtcat cgcaacgaag | |
| 301 | gttttgggaa cagtaaaatg gttcaatgta aggaacggat atggtttcat caacaggaat | |
| 361 | gacaccaagg aagatgtatt tgtacaccag actgccataa agaagaataa ccccaggaag | |
| 421 | taccttcgca gtgtaggaga tggagagact gtggagtttg atgttgttga aggagaaaag | |
| 481 | ggtgaggagg cagcaaatgt tacaggtcct ggtggtgttc cagttcaagg cagtaaatat | |
| 541 | gcagcagacc gtaaccatta tagacgctat ccacgtcgta ggggtcctcc acgcaattac | |
| 601 | cagcaaaatt accagaatag tgagagtggg gaaaagaacg agggatcgga gagtgctccc | |
| 661 | gaaggccagg cccaacaacg ccggccctac cgcaggcgaa ggttcccacc ttactacatg | |
| 721 | cggagaccct atgggcgtcg accacagtat tccaaccctc ctgtgcaggg agaagtgatg | |
| 781 | gagggtgctg acaaccaggg tgcaggagaa caaggtagac cagtgaggca gaatatgtat | |
| 841 | cggggatata gaccacgatt ccgcaggggc cctcctcgcc aaagacagcc tagagaggac | |
| 901 | ggcaatgaag aagataaaga aaatcaagga gatgagaccc aaggtcagca gccacctcaa | |
| 961 | cgtcggtacc gccgcaactt caattaccga cgcagacgcc cagaaaaccc taaaccacaa | |
| 1021 | gatggcaaag agacaaaagc agccgatcca ccagctgaga attcccgcic ccgaggctga | |
| 1081 | gcagggcggg gctgagtaaa tgccggctta ccatctctac catcalccgg tttagtcatc | |
| 1141 | caacaagaag aaatatgaaa ttccagcaat aagaaatgaa caaaagattg gagctgaaga | |
| 1201 | cctaaagtac ttgctttttg ccgtttgcaa ccagataaat agaactatct gcattatcta | |
| 1261 | tgcagcatgg ggtttatatt ttactaagac gctctttggt atacaacggt tttaaaagcc | |
| 1321 | tggttttctc aatacgcctt aaaggtttta aattgtttca tatctggtca agttgagatt | |
| 1381 | tttaagaact tcatttttaa tttgtaataa aagtttacaa cftgattttt tcaaaaaagt | |
| 1441 | caacaaactg caagcacctg ttaataaagg tcttaaataa t --3′ | |

A YB-1 polypeptide thus refers to a polypeptide that comprises a YB-1 sequence as provided herein or a variant of such a sequence. Such polypeptides are capable of binding to a PTP1B promoter Y-box protein binding site (e.g., SEQ ID NO:1), with an activity that is not substantially diminished relative to that of a full length native YB-1. YB-1 polypeptide variants within the scope of the present invention may contain one or more substitutions, deletions, additions and/or insertions. For certain YB-1 variants, the ability of the variant to bind to PTP1B −155/−132 is not substantially diminished. The ability of such a YB-1 variant to bind to PTP1B −155/−132 may be enhanced or unchanged, relative to a native YB-1, or may be diminished by less than 50%, and preferably less than 20%, relative to native YB-1. Such variants may be identified using the representative assays provided herein.

Preferably, a variant contains conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes.

In general, modifications may be more readily made in non-critical regions, which are regions of the native sequence that do not substantially change the activity of YB-1. Non-critical regions may be identified by modifying the YB-1 sequence in a particular region and assaying the ability of the resulting variant in a PTP1B −155/−132 binding assay, as described herein. Preferred sequence modifications are made so as to retain most or all of the cold-shock binding domain. Within certain preferred embodiments, such modifications affect interactions between YB-1 and cellular components other than PTP1B −155/−132. However, substitutions may also be made in critical regions of the native protein, provided that the resulting variant substantially retains the ability to bind PTP1B −155/−132. Within certain embodiments, a variant contains substitutions, deletions, additions and/or insertions at no more than 50%, preferably no more than 35%, more preferably no more than 25%, and still more preferably no more than 10% of the amino acid residues. Variants may also (or alternatively) be modified by, for example, the deletion or addition of amino acids that have minimal influence on the activity of the polypeptide. In particular, variants may contain additional amino acid sequences at the amino and/or carboxy termini. Such sequences may be used, for example, to facilitate purification or detection of the polypeptide.

YB-1 (or YB-1 alternate form) polypeptides may be prepared using any of a variety of well known techniques. Recombinant polypeptides encoded by DNA sequences as cited herein may be readily prepared from the DNA sequences using any of a variety of expression vectors known to those having ordinary skill in the art. Expression may be achieved in any appropriate host cell that has been transformed or transfected with an expression vector containing a DNA molecule that encodes a recombinant polypeptide. Suitable host cells include prokaryotes, yeast and higher eukaryotic cells (including mammalian cells), and forms that differ in glycosylation may be generated by varying the host cell or post-isolation processing. Supernatants from suitable host/vector systems which secrete recombinant protein or polypeptide into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant polypeptide.

Portions and other variants having fewer than about 100 amino acids, and generally fewer than about 50 amino acids, may also be generated by synthetic procedures, using techniques well known to those having ordinary skill in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin-Elmer, Inc., Applied BioSystems Division (Foster City, Calif.), and may be operated according to the manufacturer's instructions.

In one aspect of the present invention, Y-box proteins such as YB-1 polypeptides may be used in methods to identify agents that alter PTP1B expression and/or that impair binding (i.e., diminish binding in some material way (with statistical significance), such as by preventing binding or inhibiting binding) of a Y-box protein to a PTP1B promoter Y-box protein binding site (e.g., PTP1B −155/−132). Such agents may inhibit or enhance signal transduction via a PTP1B signal transduction pathway, leading to an altered cellular phenotype that is associated with the PTP1B pathway (e.g., insulin receptor pathway downstream signaling). An agent that alters PTP1B expression and/or that impairs Y-box protein binding activity may alter (e.g., increase or decrease in a statistically significant manner) expression and/or stability of PTP 1B, PTP1B protein activity and/or the ability of PTP1B to dephosphorylate a substrate, and in preferred embodiments as described herein the agent decreases PTP1B expression levels. Agents that may be screened within such assays include, but are not limited to, antibodies and antigen-binding fragments thereof (e.g., antibodies (which may include intrabodies as described herein) that specifically bind to a Y-box protein such as YB-1, or to any other protein that is capable of binding to PTP1B −155/−132), competing YB-1 substrates such as polynucleotides or peptides that represent, for example, a mimic of a PTP1B promoter Y-box protein binding site or that otherwise compete with PTP1B −155/−132 for YB-1 binding recognition, antisense polynucleotides and ribozymes that interfere with transcription and/or translation of YB-1 and other natural and synthetic molecules, for example small molecule inhibitors, that bind to and inactivate YB-1 or PTP1B −155/−132.

Candidate agents for use in these and related method of screening according to the present invention may be provided as “libraries” or collections of compounds, compositions or molecules. Such molecules typically include compounds known in the art as “small molecules” and having molecular weights less than 10⁵ daltons, preferably less than 10⁴ daltons and still more preferably less than 1 daltons. For example, members of a library of test compounds can be administered to a plurality of samples, each containing at least one Y-box protein or polypeptide (e.g., YB-1) and a recombinant nucleic acid construct that comprises PTP1B −155/−132 operably linked to a reporter gene as provided herein, and then assayed for their ability to enhance or inhibit transcription or expression (e.g., translation) of the reporter gene. Compounds so identified as capable of influencing PTP1B expression levels are valuable for therapeutic and/or diagnostic purposes, since they permit treatment and/or detection of PTP1B associated disorders. Such compounds are also valuable in research directed to molecular signaling mechanisms that involve PTP1B, and to refinements in the discovery and development of future PTP1B compounds exhibiting greater specificity.

Candidate agents further may be provided as members of a combinatorial library, which preferably includes synthetic agents prepared according to a plurality of predetermined chemical reactions performed in a plurality of reaction vessels. For example, various starting compounds may be prepared employing one or more of solid-phase synthesis, recorded random mix methodologies and recorded reaction split techniques that permit a given constituent to traceably undergo a plurality of permutations and/or combinations of reaction conditions. The resulting products comprise a library that can be screened followed by iterative selection and synthesis procedures, such as a synthetic combinatorial library of peptides (see e.g., PCT/US91/08694, PCT/US91/04666, which are hereby incorporated by reference in their entireties) or other compositions that may include small molecules as provided herein (see e.g, PCT/US94/08542, EP 0774464, U.S. Pat. No. 5,798,035, U.S. Pat. No. 5,789,172, U.S. Pat. No. 5,751,629, which are hereby incorporated by reference in their entireties). Those having ordinary skill in the art will appreciate that a diverse assortment of such libraries may be prepared according to established procedures, and tested using PTP1B −155/−132 and YB-1 or an equivalent, according to the present disclosure.

In certain embodiments, modulating agents may be identified by contacting a candidate agent with a sample comprising a cell that comprises a PTP1B gene and that is capable of PTP1B gene transcription or expression (e.g., translation), under conditions and for a time sufficient to detect PTP1B gene transcription or expression, and comparing PTP1B transcription levels in the absence and presence of the candidate agent. Preferably PTP1B transcription or expression is decreased in the presence of the agent, thereby providing an alternative to PTP1B active-site directed approaches to modulating PTP1B activity. (The invention need not be so limited, however, and contemplates other embodiments wherein PTP1B transcription and/or expression levels may be increased in the presence of a candidate agent.) In certain further embodiments the cell comprises an insulin receptor, such as IR-β, and the agent effects an increase in insulin receptor phosphorylation, presumably (and according to non-binding theory) by decreasing PTP1B levels through interference with the YB-1-PTP1B −155/−132 binding interaction. Methods for determining insulin receptor phosphorylation are known in the art (e.g., Cheatham et al., 1995 Endocr. Rev. 16:117-142) and are described in greater detail below. In certain other further embodiments wherein the cell comprises an insulin receptor, any of a variety of cellular insulin responses may be monitored according to art-established methodologies, including but not limited to glucose uptake (e.g., Elchebly et al., 1999 Science 283:1544; McGuire et al., 1991 Diabetes 40:939; Myerovitch et al., 1989 J. Clin. Invest. 84:976; Sredy et al. 1995 Metabolism 44:1074; WO 99/46268); glycogen synthesis (e.g., Berger et al., 1998 Anal. Biochem. 261:159), Glut4 recruitment to a plasma membrane (Robinson et al., 1992 J. Cell Biol. 117:1181); or amino acid import (Hyde et al., 2002 J. Biol. Chem. Online, “Insulin promotes the cell surface recruitment of the SAT2/ATA2 system A amino acid transporter from an endosomal compartment in skeletal muscle cells”). In certain other further embodiments wherein the cell comprises an insulin receptor, cellular insulin responses that may be monitored include MAP kinase phosphorylation (see Examples), AKT phosphorylation (see Examples), inhibition of phosphoenolpyruvate carboxykinase transcription (Forest et al., 1990 Molec. Endocrinol. 4:1302), phosphatidylinositoltriphosphate kinase activation (Endeman et al., 1990 J. Biol. Chem. 265:396), lipogenesis (Moody et al., 1974 Horm. Metab. Res. 6:12), lipolysis (Hess et al., 1991 J. Cell. Biochem. 45:374), TYK2 dephosphorylation and JAK2 dephosphorylation (Myers et al., 2001 J. Biol. Chem. 276:47771) and EGF or PDGRF phosphorylation (Ullrich et al., 1990 Cell 61:203).

PTP1B activity may also be measured in whole cells transfected with a reporter gene whose expression is dependent upon the activation of an appropriate substrate. For example, appropriate cells (i.e., cells that express PTP1B and a Y-box protein such as YB-1) may be transfected with a substrate-dependent promoter linked to a reporter gene. In such a system, expression of the reporter gene (which may be readily detected using methods well known to those of ordinary skill in the art) depends upon activation of substrate. Dephosphorylation of substrate may be detected based on a decrease in reporter activity.

Candidate modulating agents may be added to such a system, as described above, to evaluate their effect on PTP1B activity.

The present invention further provides methods for identifying a molecule that interacts with, or binds to, PTP1B −155/−132, or that interacts with, or binds to, YB-1. Such a molecule generally associates with PTP1B −155/−132 or with YB-1 with an affinity constant (Kₐ) of at least 10⁴ M⁻¹, preferably at least 10⁵ M⁻¹,more preferably at least 10⁶ M⁻¹, still more preferably at least 10⁷ M⁻¹ and most preferably at least 10⁸ M⁻¹. Affinity constants may be determined using well known techniques. Methods for identifying interacting molecules may be used, for example, as initial screens for modulating agents, or to identify factors that are involved in the in vivo PTP1B activity. In addition to standard binding assays, there are many other techniques that are well known for identifying interacting molecules, including yeast two-hybrid screens, phage display and affinity techniques. Such techniques may be performed using routine protocols, which are well known to those having ordinary skill in the art (see, e.g., Bartel et al., In Cellular Interactions in Development: A Practical Approach, D. A. Harley, ed., Oxford University Press (Oxford, UK), pp. 153-179, 1993).

Within other aspects, the present invention provides animal models in which an animal either does not express (or expresses a significantly reduced amount of) a functional YB-1 or other PTP1B −155/−132 binding protein, or has had the PTP1B −155/−132 sequence deleted from the PTP1B upstream promoter region using genetic knockout techniques as known to the art. Such animals may be generated, for example, using standard homologous recombination strategies. Animal models generated in this manner may be used to study activities of PTP1B signaling pathway components and modulating agents in vivo.

As also described above, a nucleic acid encoding PTP1B may be detected, using standard hybridization or PCR techniques. Suitable probes and primers may be designed by those having ordinary skill in the art based on the known PTP1B cDNA sequence. For example, expression of mRNA may be determined according to methods that measure steady state mRNA levels, such as ribonuclease protection, primer extension, northern blotting, and real-time PCR (see Sambrook et al., Molecular Cloning: A Laboratory Manual (3^{rd} ed. 2001)). These and other assays described herein may generally be performed using any of a variety of samples obtained from a biological source, such as eukaryotic cells, bacteria, viruses, extracts prepared from such organisms and fluids found within living organisms. A patient or biological source may be a human or non-human animal, a primary cell culture or culture adapted cell line including but not limited to genetically engineered cell lines that may contain chromosomally integrated or episomal recombinant nucleic acid sequences, immortalized or immortalizable cell lines, somatic cell hybrid cell lines, differentiated or differentiatable cell lines, transformed cell lines and the like.

Certain embodiments of the present invention provide methods that employ antibodies raised against PTP1B, or hybridizing polynucleotides, for diagnostic and assay purposes. Certain assays involve using an antibody or other agent to detect the presence or absence of PTP1B, or proteolytic fragments thereof.

Transcriptional activity of the PTP1B gene and regulation of its expression can be analyzed by methods known in the art. For example, primary transcripts may be analyzed by transcriptional run-on, which measures the intensity of transcription of a target gene under different experimental conditions (see Sambrook et al., Molecular Cloning: A Laboratory Manual (3^{rd} ed. 2001). Typically, nuclei from cells expressing the PTP1B gene are isolated and then incubated with a radiolabeled nucleotide, such as [³²P]UTP. The transcriptional activity of the target gene is measured by hybridizing the radiolabeled RNAs to an excess of the target gene nucleic acid immobilized on a surface, for example, nitrocellulose or nylon membrane. The fraction of radioactivity that hybridizes to the immobilized DNA reflects the contribution of the target gene to the total transcriptional activity of the cell. Regulation of the gene is consequently detected by an increase or decrease in hybridization.

Reporter genes or markers may be used to identify regulatory elements of a gene and to study promoters and enhancers and their interactions with cis-acting elements and trans-acting proteins, as well as measuring the effect of the addition of agents (see Sambrook et al., supra)). A regulatory sequence of interest is joined (operably linked) to a reporter gene sequence present in an expression vector by standard recombinant methods. The resulting reporter gene recombinant is then introduced into an appropriate host cell line, and its expression is detected by measuring the reporter mRNA or the reporter protein, or in the instance when an enzyme reporter is used, by assaying for the relevant catalytic activity. The effect of the regulatory element on transcription is determined by measuring the detection signal or output that is distinguishable from background expression of proteins in the host cell. Appropriate controls for transcription assays are known in the art and may be incorporated according to the reporter system used. Suitable reporter genes may include genes encoding luciferase, beta-galactosidase, chloramphenicol acetyl transferase, secreted alkaline phosphatase, or in a non-enzymatic reporter system, green fluorescent protein (GFP) gene. Suitable expression systems are well known and may be prepared using well known techniques or obtained commercially (see Sambrook et al., supra)).

In another aspect, the present invention relates to host cells containing any of the above described recombinant nucleic acid constructs comprising a polynucleotide having the sequence set forth in SEQ ID NO:1, or a complementary sequence thereto, or any variant of the sequence, or any antisense polynucleotide that is complementary to a polynucleotide that encodes a YB-1 protein, as discussed herein. Host cells are genetically engineered (transduced, transformed or transfected) with the vectors and/or expression constructs of this invention that may be, for example, a cloning vector, a shuttle vector, or an expression construct. Genetically engineered host cells may be prepared by methods known to the skilled artisan (see, e.g., Sambrook et al., supra). The vector or construct may be, for example, in the form of a plasmid, a viral particle, a phage, linear DNA, naked DNA, and condensed DNA, or the like. The engineered host cells can be cultured in conventional nutrient media modified as appropriate for activating promoters and selecting transformants. The culture conditions for particular host cells selected for expression, such as temperature, pH, and the like, will be readily apparent to the ordinarily skilled artisan.

As described above, the subject invention includes compositions capable of delivering nucleic acid molecules, including polynucleotide sequences that are operably linked to a reporter gene. Such compositions include recombinant viral vectors (e.g., retroviruses (see WO 90/07936, WO 91/02805, WO 93/25234, WO 93/25698, and WO 94/03622), adenovirus (see Berkner, Biotechniques 6:616-627, 1988; Li et al., Hum. Gene Ther. 4:403-409, 1993; Vincent et al., Nat. Genet. 5:130-134, 1993; and Kolls et al., Proc. Natl. Acad. Sci. USA 91:215-219, 1994), pox virus (see U.S. Pat. No. 4,769,330; U.S. Pat. No. 5,017,487; and WO 89/01973)), recombinant expression construct nucleic acid molecules complexed to a polycationic molecule (see WO 93/03709), and nucleic acids associated with liposomes (see Wang et al., Proc. Natl. Acad. Sci. USA 84:7851, 1987). In certain embodiments, the DNA may be linked to killed or inactivated adenovirus (see Curiel et al., Hum. Gene Ther. 3:147-154, 1992; Cotton et al., Proc. Natl. Acad. Sci. USA 89:6094, 1992). Other suitable compositions include DNA-ligand (see Wu et al., J. Biol. Chem. 264:16985-16987, 1989) and lipid-DNA combinations (see Felgner et al., Proc. Natl. Acad. Sci. USA 84:7413-7417, 1989).

According to certain embodiments of the present invention peptides, polypeptides, and other non-peptide molecules that specifically bind to a Y-box protein may be employed. In particularly preferred embodiments the Y-box protein may be a YB-1 protein as provided herein. As used herein, a molecule is said to “specifically bind” to a Y-box protein if it reacts at a detectable level with Y-box protein, but does not react detectably with peptides containing an unrelated sequence, or a sequence of a different phosphatase. Preferred binding molecules include antibodies, which may be, for example, polyclonal, monoclonal, single chain, chimeric, anti-idiotypic, or CDR-grafted immunoglobulins, or fragments thereof, such as proteolytically generated or recombinantly produced immunoglobulin F(ab′)₂, Fab, Fv, and Fd fragments. Certain preferred antibodies are those antibodies that inhibit or block Y-box protein activity within an in vitro assay, as described herein. Binding properties of an antibody to Y-box protein may generally be assessed using immunodetection methods including, for example, an enzyme-linked immunosorbent assay (ELISA), immunoprecipitation, immunoblotting and the like, which may be readily performed by those having ordinary skill in the art.

Methods well known in the art may be used to generate antibodies, polyclonal antisera or monoclonal antibodies that are specific for a Y-box protein (e.g., Bargou et al., 1997 Nat. Med. 3:447). Antibodies also may be produced as genetically engineered immunoglobulins (Ig) or Ig fragments designed to have desirable properties. For example, by way of illustration and not limitation, antibodies may include a recombinant IgG that is a chimeric fusion protein having at least one variable (V) region domain from a first mammalian species and at least one constant region domain from a second, distinct mammalian species. Most commonly, a chimeric antibody has murine variable region sequences and human constant region sequences. Such a murine/human chimeric immunoglobulin may be “humanized” by grafting the complementarity determining regions (CDRs) derived from a murine antibody, which confer binding specificity for an antigen, into human-derived V region framework regions and human-derived constant regions. Fragments of these molecules may be generated by proteolytic digestion, or optionally, by proteolytic digestion followed by mild reduction of disulfide bonds and alkylation. Alternatively, such fragments may also be generated by recombinant genetic engineering techniques.

As used herein, an antibody is said to be “immunospecific” or to “specifically bind” a Y-box polypeptide if it reacts at a detectable level with Y-box protein, preferably with an affinity constant, Kₐ, of greater than or equal to about 10⁴ M⁻¹, more preferably of greater than or equal to about 10⁵ M⁻¹, more preferably of greater than or equal to about 10⁶ M⁻¹, and still more preferably of greater than or equal to about 10⁷ M⁻¹. Affinities of binding partners or antibodies can be readily determined using conventional techniques, for example, those described by Scatchard et al. (Ann. N.Y. Acad. Sci. USA 51:660 (1949)) or by surface plasmon resonance (BIAcore, Biosensor, Piscataway, N.J.). For surface plasmon resonance, target molecules are immobilized on a solid phase and exposed to ligands in a mobile phase running along a flow cell. If ligand binding to the immobilized target occurs, the local refractive index changes, leading to a change in SPR angle, which can be monitored in real time by detecting changes in the intensity of the reflected light. The rates of change of the surface plasmon resonance signal can be analyzed to yield apparent rate constants for the association and dissociation phases of the binding reaction. The ratio of these values gives the apparent equilibrium constant (affinity). See, e.g., Wolff et al., Cancer Res. 53:2560-2565 (1993).

Antibodies may generally be prepared by any of a variety of techniques known to those having ordinary skill in the art. See, e.g., Harlow et al., Antibodies: A Laboratory Manual, Cold Spring Harbor Laboratory (1988). In one such technique, an animal is immunized with Y-box protein as an antigen to generate polyclonal antisera. Suitable animals include, for example, rabbits, sheep, goats, pigs, cattle, and may also include smaller mammalian species, such as mice, rats, and hamsters, or other species.

An immunogen may be comprised of cells expressing Y-box protein, purified or partially purified Y-box protein polypeptides or variants or fragments (e.g., peptides) thereof, or Y-box protein peptides. Y-box protein peptides may be generated by proteolytic cleavage or may be chemically synthesized. For instance, nucleic acid sequences encoding Y-box protein polypeptides are provided herein, such that those skilled in the art may routinely prepare these polypeptides for use as immunogens. Polypeptides or peptides useful for immunization may also be selected by analyzing the primary, secondary, and tertiary structure of Y-box protein according to methods known to those skilled in the art, in order to determine amino acid sequences more likely to generate an antigenic response in a host animal. See, e.g., Novotny, 1991 Mol. Immunol. 28:201-207; Berzofsky, 1985 Science 229:932-40.

Preparation of the immunogen for injection into animals may include covalent coupling of the Y-box protein polypeptide (or variant or fragment thereof), to another immunogenic protein, for example, a carrier protein such as keyhole limpet hemocyanin (KLH) or bovine serum albumin (BSA). In addition, the Y-box protein peptide, polypeptide, or Y-box protein-expressing cells to be used as immunogen may be emulsified in an adjuvant. See, e.g., Harlow et al., Antibodies: A Laboratory Manual, Cold Spring Harbor Laboratory (1988). In general, after the first injection, animals receive one or more booster immunizations according to a preferred schedule that may vary according to, inter alia, the antigen, the adjuvant (if any) and/or the particular animal species. The immune response may be monitored by periodically bleeding the animal, separating the sera out of the collected blood, and analyzing the sera in an immunoassay, such as an ELISA or Ouchterlony diffusion assay, or the like, to determine the specific antibody titer. Once an antibody titer is established, the animals may be bled periodically to accumulate the polyclonal antisera. Polyclonal antibodies that bind specifically to the Y-box protein polypeptide or peptide may then be purified from such antisera, for example, by affinity chromatography using protein A, or the Y-box protein polypeptide, immobilized on a suitable solid support.

Monoclonal antibodies that specifically bind to Y-box protein polypeptides or fragments or variants thereof, and hybridomas, which are immortal eukaryotic cell lines, that produce monoclonal antibodies having the desired binding specificity, may also be prepared, for example, using the technique of Kohler and Milstein (Nature, 256:495-497; 1976, Eur. J. Immunol. 6:511-519 (1975)) and improvements thereto. An animal—for example, a rat, hamster, or preferably mouse—is immunized with a Y-box protein immunogen prepared as described above. Lymphoid cells that include antibody-forming cells, typically spleen cells, are obtained from an immunized animal and may be immortalized by fusion with a drug-sensitized myeloma (e.g., plasmacytoma) cell fusion partner, preferably one that is syngeneic with the immunized animal and that optionally has other desirable properties (e.g., inability to express endogenous Ig gene products). The lymphoid (e.g., spleen) cells and the myeloma cells may be combined for a few minutes with a membrane fusion-promoting agent, such as polyethylene glycol or a nonionic detergent, and then plated at low density on a selective medium that supports the growth of hybridoma cells, but not unfused myeloma cells. A preferred selection media is HAT (hypoxanthine, aminopterin, thymidine). After a sufficient time, usually about one to two weeks, colonies of cells are observed. Single colonies are isolated, and antibodies produced by the cells may be tested for binding activity to the Y-box protein polypeptide, or variant or fragment thereof. Hybridomas producing monoclonal antibodies with high affinity and specificity for a Y-box protein antigen are preferred. Hybridomas that produce monoclonal antibodies that specifically bind to a Y-box protein polypeptide or variant or fragment thereof are therefore contemplated by the present invention.

Monoclonal antibodies may be isolated from the supernatants of hybridoma cultures. An alternative method for production of a murine monoclonal antibody is to inject the hybridoma cells into the peritoneal cavity of a syngeneic mouse, for example, a mouse that has been treated (e.g., pristane-primed) to promote formation of ascites fluid containing the monoclonal antibody. Contaminants may be removed from the subsequently (usually within 1-3 weeks) harvested ascites fluid by conventional techniques, such as chromatography, gel filtration, precipitation, extraction, or the like. For example, antibodies may be purified by affinity chromatography using an appropriate ligand selected based on particular properties of the monoclonal antibody (e.g., heavy or light chain isotype, binding specificity, etc.). Examples of a suitable ligand, immobilized on a solid support, include Protein A, Protein G, an anti-constant region (light chain or heavy chain) antibody, an anti-idiotype antibody and a Y-box protein polypeptide or fragment or variant thereof.

Human monoclonal antibodies may be generated by any number of techniques with which those having ordinary skill in the art will be familiar. Such methods include but are not limited to, Epstein Barr Virus (EBV) transformation of human peripheral blood cells (e.g., containing B lymphocytes), in vitro immunization of human B cells, fusion of spleen cells from immunized transgenic mice carrying human immunoglobulin genes inserted by yeast artificial chromosomes (YAC), isolation from human immunoglobulin V region phage libraries, or other procedures as known in the art and based on the disclosure herein.

For example, one method for generating human monoclonal antibodies includes immortalizing human peripheral blood cells by EBV transformation. See, e.g., U.S. Pat. No. 4,464,456. An immortalized cell line producing a monoclonal antibody that specifically binds to a Y-box protein polypeptide (or a variant or fragment thereof) can be identified by immunodetection methods as provided herein, for example, an ELISA, and then isolated by standard cloning techniques. Another method to generate human monoclonal antibodies, in vitro immunization, includes priming human splenic B cells with antigen, followed by fusion of primed B cells with a heterohybrid fusion partner. See, e.g., Boemer et al., 1991 J. Immunol. 147:86-95.

Still another method for the generation of human Y-box protein-specific monoclonal antibodies and polyclonal antisera for use in the present invention relates to transgenic mice. See, e.g., U.S. Pat. No. 5,877,397; Bruggemann et al., 1997 Curr. Opin. Biotechnol. 8:455-58; Jakobovits et al., 1995 Ann. N.Y. Acad. Sci. 764:525-35. In these mice, human immunoglobulin heavy and light chain genes have been artificially introduced by genetic engineering in germline configuration, and the endogenous murine immunoglobulin genes have been inactivated. See, e.g., Bruggemann et al., 1997 Curr. Opin. Biotechnol. 8:455-58. For example, human immunoglobulin transgenes may be mini-gene constructs, or transloci on yeast artificial chromosomes, which undergo B cell-specific DNA rearrangement and hypermutation in the mouse lymphoid tissue. See, Bruggemann et al., 1997 Curr. Opin. Biotechnol. 8:455-58. Human monoclonal antibodies specifically binding to Y-box protein may be obtained by immunizing the transgenic animals, fusing spleen cells with myeloma cells, selecting and then cloning cells producing antibody, as described above. Polyclonal sera containing human antibodies may also be obtained from the blood of the immunized animals.

Chimeric antibodies, specific for a Y-box protein, including humanized antibodies, may also be generated according to the present invention. A chimeric antibody has at least one constant region domain derived from a first mammalian species and at least one variable region domain derived from a second, distinct mammalian species. See, e.g., Morrison et al., 1984, Proc. Natl. Acad. Sci. USA, 81:6851-55. In preferred embodiments, a chimeric antibody may be constructed by cloning the polynucleotide sequence that encodes at least one variable region domain derived from a non-human monoclonal antibody, such as the variable region derived from a murine, rat, or hamster monoclonal antibody, into a vector containing a nucleic acid sequence that encodes at least one human constant region. See, e.g., Shin et al., 1989 Methods Enzymol. 178:459-76; Walls et al., 1993 Nucleic Acids Res. 21:2921-29. By way of example, the polynucleotide sequence encoding the light chain variable region of a murine monoclonal antibody may be inserted into a vector containing a nucleic acid sequence encoding the human kappa light chain constant region sequence. In a separate vector, the polynucleotide sequence encoding the heavy chain variable region of the monoclonal antibody may be cloned in frame with sequences encoding the human IgG1 constant region. The particular human constant region selected may depend upon the effector functions desired for the particular antibody (e.g., complement fixing, binding to a particular Fe receptor, etc.). Another method known in the art for generating chimeric antibodies is homologous recombination (e.g., U.S. Pat. No. 5,482,856). Preferably, the vectors will be transfected into eukaryotic cells for stable expression of the chimeric antibody.

A non-human/human chimeric antibody may be further genetically engineered to create a “humanized” antibody. Such a humanized antibody may comprise a plurality of CDRs derived from an immunoglobulin of a non-human mammalian species, at least one human variable framework region, and at least one human immunoglobulin constant region. Humanization may in certain embodiments provide an antibody that has decreased binding affinity for a Y-box protein when compared, for example, with either a non-human monoclonal antibody from which a Y-box protein binding variable region is obtained, or a chimeric antibody having such a V region and at least one human C region, as described above. Useful strategies for designing humanized antibodies may therefore include, for example by way of illustration and not limitation, identification of human variable framework regions that are most homologous to the non-human framework regions of the chimeric antibody. Without wishing to be bound by theory, such a strategy may increase the likelihood that the humanized antibody will retain specific binding affinity for a Y-box protein, which in some preferred embodiments may be substantially the same affinity for a Y-box protein polypeptide or variant or fragment thereof, and in certain other preferred embodiments may be a greater affinity for Y-box protein. See, e.g., Jones et al., 1986 Nature 321:522-25; Riechmann et al., 1988 Nature 332:323-27. Designing such a humanized antibody may therefore include determining CDR loop conformations and structural determinants of the non-human variable regions, for example, by computer modeling, and then comparing the CDR loops and determinants to known human CDR loop structures and determinants. See, e.g., Padlan et al., 1995 FASEB 9:133-39; Chothia et al., 1989 Nature, 342:377-383. Computer modeling may also be used to compare human structural templates selected by sequence homology with the non-human variable regions. See, e.g., Bajorath et al., 1995 Ther. Immunol. 2:95-103; EP-0578515-A3. If humanization of the non-human CDRs results in a decrease in binding affinity, computer modeling may aid in identifying specific amino acid residues that could be changed by site-directed or other mutagenesis techniques to partially, completely or supra-optimally (i.e., increase to a level greater than that of the non-humanized antibody) restore affinity. Those having ordinary skill in the art are familiar with these techniques, and will readily appreciate numerous variations and modifications to such design strategies.

Within certain embodiments, the use of antigen-binding fragments of antibodies may be preferred. Such fragments include Fab fragments or F(ab′)₂ fragments, which may be prepared by proteolytic digestion with papain or pepsin, respectively. The antigen binding fragments may be separated from the Fc fragments by affinity chromatography, for example, using immobilized protein A or protein G, or immobilized Y-box protein polypeptide, or a suitable variant or fragment thereof. Those having ordinary skill in the art can routinely and without undue experimentation determine what is a suitable variant or fragment based on characterization of affinity purified antibodies obtained, for example, using immunodetection methods as provided herein. An alternative method to generate Fab fragments includes mild reduction of F(ab′)₂ fragments followed by alkylation. See, e.g., Weir, Handbook of Experimental Immunology, 1986, Blackwell Scientific, Boston.

According to certain embodiments, non-human, human, or humanized heavy chain and light chain variable regions of any of the above described Ig molecules may be constructed as single chain Fv (sFv) polypeptide fragments (single chain antibodies). See, e.g., Bird et al., 1988 Science 242:423-426; Huston et al., 1988 Proc. Natl. Acad. Sci. USA 85:5879-5883. Multi-functional sFv fusion proteins may be generated by linking a polynucleotide sequence encoding an sFv polypeptide in-frame with at least one polynucleotide sequence encoding any of a variety of known effector proteins. These methods are known in the art, and are disclosed, for example, in EP-BI-0318554, U.S. Pat. No. 5,132,405, U.S. Pat. No. 5,091,513, and U.S. Pat. No. 5,476,786. By way of example, effector proteins may include immunoglobulin constant region sequences. See, e.g., Hollenbaugh et al., 1995 J. Immunol. Methods 188:1-7. Other examples of effector proteins are enzymes. As a non-limiting example, such an enzyme may provide a biological activity for therapeutic purposes (see, e.g., Siemers et al., 1997 Bioconjug. Chem. 8:510-19), or may provide a detectable activity, such as horseradish peroxidase-catalyzed conversion of any of a number of well-known substrates into a detectable product, for diagnostic uses. Still other examples of sFv fusion proteins include Ig-toxin fusions, or immunotoxins, wherein the sFv polypeptide is linked to a toxin. Those having ordinary skill in the art will appreciate that a wide variety of polypeptide sequences have been identified that, under appropriate conditions, are toxic to cells. As used herein, a toxin polypeptide for inclusion in an immunoglobulin-toxin fusion protein may be any polypeptide capable of being introduced to a cell in a manner that compromises cell survival, for example, by directly interfering with a vital function or by inducing apoptosis. Toxins thus may include, for example, ribosome-inactivating proteins, such as Pseudomonas aeruginosa exotoxin A, plant gelonin, bryodin from Bryonia dioica, or the like. See, e.g., Thrush et al., 1996 Annu. Rev. Immunol. 14:49-71; Frankel et al., 1996 Cancer Res. 56:926-32. Numerous other toxins, including chemotherapeutic agents, anti-mitotic agents, antibiotics, inducers of apoptosis (or “apoptogens”, see, e.g., Green and Reed, 1998, Science 281:1309-1312), or the like, are known to those familiar with the art, and the examples provided herein are intended to be illustrative without limiting the scope and spirit of the invention.

The sFv may, in certain embodiments, be fused to peptide or polypeptide domains that permit detection of specific binding between the fusion protein and antigen (e.g., a Y-box protein). For example, the fusion polypeptide domain may be an affinity tag polypeptide. Binding of the sFv fusion protein to a binding partner (e.g., a Y-box protein) may therefore be detected using an affinity polypeptide or peptide tag, such as an avidin, streptavidin or a His (e.g., polyhistidine) tag, by any of a variety of techniques with which those skilled in the art will be familiar. Detection techniques may also include, for example, binding of an avidin or streptavidin fusion protein to biotin or to a biotin mimetic sequence (see, e.g., Luo et al., 1998 J. Biotechnol. 65:225 and references cited therein), direct covalent modification of a fusion protein with a detectable moiety (e.g., a labeling moiety), non-covalent binding of the fusion protein to a specific labeled reporter molecule, enzymatic modification of a detectable substrate by a fusion protein that includes a portion having enzyme activity, or immobilization (covalent or non-covalent) of the fusion protein on a solid-phase support.

The sFv fusion protein of the present invention, comprising a Y-box protein-specific immunoglobulin-derived polypeptide fused to another polypeptide such as an effector peptide having desirable affinity properties, may therefore include, for example, a fusion protein wherein the effector peptide is an enzyme such as glutathione-S-transferase. As another example, sFv fusion proteins may also comprise a Y-box protein-specific Ig polypeptide fused to a Staphylococcus aureus protein A polypeptide; protein A encoding nucleic acids and their use in constructing fusion proteins having affinity for immunoglobulin constant regions are disclosed generally, for example, in U.S. Pat. No. 5,100,788. Other useful affinity polypeptides for construction of sFv fusion proteins may include streptavidin fusion proteins, as disclosed, for example, in WO 89/03422; U.S. Pat. No. 5,489,528; U.S. Pat. No. 5,672,691; WO 93/24631; U.S. Pat. No. 5,168,049; U.S. Pat. No. 5,272,254 and elsewhere, and avidin fusion proteins (see, e.g., EP 511,747). As provided herein, sFv polypeptide sequences may be fused to fusion polypeptide sequences, including effector protein sequences, that may include full length fusion polypeptides and that may alternatively contain variants or fragments thereof.

An additional method for selecting antibodies that specifically bind to a Y-box protein polypeptide or variant or fragment thereof is by phage display. See, e.g., Winter et al., 1994 Annu. Rev. Immunol. 12:433-55; Burton et al., 1994 Adv. Immunol. 57:191-280. Human or murine immunoglobulin variable region gene combinatorial libraries may be created in phage vectors that can be screened to select Ig fragments (Fab, Fv, sFv, or multimers thereof) that bind specifically to a Y-box protein polypeptide or variant or fragment thereof. See, e.g., U.S. Pat. No. 5,223,409; Huse et al., 1989 Science 246:1275-81; Kang et al., 1991 Proc. Natl. Acad. Sci. USA 88:4363-66; Hoogenboom et al., 1992 J. Molec. Biol. 227:381-388; Schlebusch et al., 1997 Hybridoma 16:47-52 and references cited therein. For example, a library containing a plurality of polynucleotide sequences encoding Ig variable region fragments may be inserted into the genome of a filamentous bacteriophage, such as M13 or a variant thereof, in frame with the sequence encoding a phage coat protein, for instance, gene III or gene VIII of M13, to create an M13 fusion protein. A fusion protein may be a fusion of the coat protein with the light chain variable region domain and/or with the heavy chain variable region domain.

According to certain embodiments, immunoglobulin Fab fragments may also be displayed on the phage particle, as follows. Polynucleotide sequences encoding Ig constant region domains may be inserted into the phage genome in frame with a coat protein. The phage coat fusion protein may thus be fused to an Ig light chain or heavy chain fragment (Fd). For example, from a human Ig library, the polynucleotide sequence encoding the human kappa constant region may be inserted into a vector in frame with the sequence encoding at least one of the phage coat proteins. Additionally or alternatively, the polynucleotide sequence encoding the human IgG1 CHI domain may be inserted in frame with the sequence encoding at least one other of the phage coat proteins. A plurality of polynucleotide sequences encoding variable region domains (e.g., derived from a DNA library) may then be inserted into the vector in frame with the constant region-coat protein fusions, for expression of Fab fragments fused to a bacteriophage coat protein.

Phage that display an Ig fragment (e.g., an Ig V-region or Fab) that binds to a Y-box protein polypeptide may be selected by mixing the phage library with Y-box protein or a variant or a fragment thereof, or by contacting the phage library with a Y-box protein polypeptide immobilized on a solid matrix under conditions and for a time sufficient to allow binding. Unbound phage are removed by a wash, which typically may be a buffer containing salt (e.g., NaCl) at a low concentration, preferably with less than 100 mM NaCl, more preferably with less than 50 mM NaCl, most preferably with less than 10 mM NaCl, or, alternatively, a buffer containing no salt. Specifically bound phage are then eluted with an NaCl-containing buffer, for example, by increasing the salt concentration in a step-wise manner. Typically, phage that bind the Y-box protein with higher affinity will require higher salt concentrations to be released. Eluted phage may be propagated in an appropriate bacterial host, and generally, successive rounds of Y-box protein binding and elution can be repeated to increase the yield of phage expressing Y-box protein specific immunoglobulin. Combinatorial phage libraries may also be used for humanization of non-human variable regions. See, e.g., Rosok et al., 1996 J. Biol. Chem. 271:22611-18; Rader et al., 1998 Proc. Natl. Acad. Sci. USA 95:8910-15. The DNA sequence of the inserted immunoglobulin gene in the phage so selected may be determined by standard techniques. See, Sambrook et al., supra. The affinity selected Ig-encoding sequence may then be cloned into another suitable vector for expression of the Ig fragment or, optionally, may be cloned into a vector containing Ig constant regions, for expression of whole immunoglobulin chains.

Phage display techniques may also be used to select polypeptides, peptides or single chain antibodies that bind to Y-box protein. For examples of suitable vectors having multicloning sites into which candidate nucleic acid molecules (e.g., DNA) encoding such peptides or antibodies may be inserted, see, e.g., McLafferty et al., Gene 128:29-36, 1993; Scott et al., 1990 Science 249:386-390; Smith et al., 1993 Methods Enzymol. 217:228-257; Fisch et al., 1996, Proc. Natl. Acad. Sci. USA 93:7761-66. The inserted DNA molecules may comprise randomly generated sequences, or may encode variants of a known peptide or polypeptide domain that specifically binds to a Y-box protein polypeptide, or variant or fragment thereof, as provided herein. Generally, the nucleic acid insert encodes a peptide of up to 60 amino acids, more preferably a peptide of 3 to 35 amino acids, and still more preferably a peptide of 6 to 20 amino acids. The peptide encoded by the inserted sequence is displayed on the surface of the bacteriophage. Phage expressing a binding domain for a Y-box protein polypeptide may be selected on the basis of specific binding to an immobilized Y-box protein polypeptide as described above. As provided herein, well-known recombinant genetic techniques may be used to construct fusion proteins containing the fragment thereof. For example, a polypeptide may be generated that comprises a tandem array of two or more similar or dissimilar affinity selected Y-box protein binding peptide domains, in order to maximize binding affinity for Y-box protein of the resulting product.

In certain other embodiments, the invention contemplates Y-box protein specific antibodies that are multimeric antibody fragments. Useful methodologies are described generally, for example in Hayden et al. 1997, Curr Opin. Immunol. 9:201-12; Coloma et al., 1997 Nat. Biotechnol. 15:159-63). For example, multimeric antibody fragments may be created by phage techniques to form miniantibodies (U.S. Pat. No. 5,910,573) or diabodies (Holliger et al., 1997, Cancer Immunol. Immunother. 45:128-130). Multimeric fragments may be generated that are multimers of a Y-box protein-specific Fv, or that are bispecific antibodies comprising a Y-box protein-specific Fv noncovalently associated with a second Fv having a different antigen specificity. See, e.g., Koelemij et al., 1999 J. Immunother. 22:514-24. As another example, a multimeric antibody may comprise a bispecific antibody having two single chain antibodies or Fab fragments. According to certain related embodiments, a first Ig fragment may be specific for a first antigenic determinant on a Y-box protein polypeptide (or variant or fragment thereof), while a second Ig fragment may be specific for a second antigenic determinant of the Y-box protein polypeptide. Alternatively, in certain other related embodiments, a first immunoglobulin fragment may be specific for an antigenic determinant on a Y-box protein polypeptide or variant or fragment thereof, and a second immunoglobulin fragment may be specific for an antigenic determinant on a second, distinct (i.e., non-Y-box protein) molecule. Also contemplated are bispecific antibodies that specifically bind Y-box protein, wherein at least one antigen-binding domain is present as a fusion protein.

In certain embodiments of the invention, an antibody specific for a YB-1 polypeptide may be an antibody that is expressed as an intracellular protein. Such intracellular antibodies are also referred to as intrabodies and may comprise an Fab fragment, or preferably comprise a single chain Fv (scFv) fragment (see, e.g., Lecerfet al., Proc. Natl. Acad. Sci. USA 98:4764-49 (2001). The framework regions flanking the CDR regions can be modified to improve expression levels and solubility of an intrabody in an intracellular reducing environment (see, e.g., Worn et al., J. Biol. Chem. 275:2795-803 (2000). An intrabody may be directed to a particular cellular location or organelle, for example by constructing a vector that comprises a polynucleotide sequence encoding the variable regions of an intrabody that may be operatively fused to a polynucleotide sequence that encodes a particular target antigen within the cell (see, e.g., Graus-Porta et al., Mol. Cell Biol. 15:1182-91 (1995); Lener et al., Eur. J. Biochem. 267:1196-205 (2000)). An intrabody may be introduced into a cell by a variety of techniques available to the skilled artisan including via a gene therapy vector, or a lipid mixture (e.g., Provectin™ manufactured by Imgenex Corporation, San Diego, Calif.), or according to photochemical internalization methods.

Introducing amino acid mutations into Y-box protein-binding immunoglobulin molecules may be useful to increase the specificity or affinity for Y-box protein, or to alter an effector function. Immunoglobulins with higher affinity for Y-box protein may be generated by site-directed mutagenesis of particular residues. Computer assisted three-dimensional molecular modeling may be employed to identify the amino acid residues to be changed, in order to improve affinity for the Y-box protein polypeptide. See, e.g., Mountain et al., 1992, Biotechnol. Genet. Eng. Rev. 10: 1-142. Alternatively, combinatorial libraries of CDRs may be generated in M13 phage and screened for immunoglobulin fragments with improved affinity. See, e.g., Glaser et al., 1992, J. Immunol. 149:3903-3913; Barbas et al., 1994 Proc. Natl. Acad. Sci. USA 91:3809-13; U.S. Pat. No. 5,792,456).

Effector functions may also be altered by site-directed mutagenesis. See, e.g., Duncan et al., 1988 Nature 332:563-64; Morgan et al., 1995 Immunology 86:319-24; Eghtedarzedeh-Kondri et al., 1997 Biotechniques 23:830-34. For example, mutation of the glycosylation site on the Fc portion of the immunoglobulin may alter the ability of the immunoglobulin to fix complement. See, e.g., Wright et al., 1997 Trends Biotechnol. 15:26-32. Other mutations in the constant region domains may alter the ability of the immunoglobulin to fix complement, or to effect antibody-dependent cellular cytotoxicity. See, e.g., Duncan et al., 1988 Nature 332:563-64; Morgan et al., 1995 Immunology 86:319-24; Sensel et al., 1997 Mol. Immunol. 34:1019-29.

The nucleic acid molecules encoding an antibody or fragment thereof that specifically binds Y-box protein, as described herein, may be propagated and expressed according to any of a variety of well-known procedures for nucleic acid excision, ligation, transformation and transfection. Thus, in certain embodiments expression of an antibody fragment may be preferred in a prokaryotic host, such as Escherichia coli (see, e.g., Pluckthun et al., 1989 Methods Enzymol. 178:497-515). In certain other embodiments, expression of the antibody or a fragment thereof may be preferred in a eukaryotic host cell, including yeast (e.g, Saccharomyces cerevisiae, Schizosaccharomyces pombe, and Pichia pastoris), animal cells (including mammalian cells) or plant cells. Examples of suitable animal cells include, but are not limited to, myeloma, COS, CHO, or hybridoma cells. Examples of plant cells include tobacco, corn, soybean, and rice cells. By methods known to those having ordinary skill in the art and based on the present disclosure, a nucleic acid vector may be designed for expressing foreign sequences in a particular host system, and then polynucleotide sequences encoding the Y-box protein binding antibody (or fragment thereof) may be inserted. The regulatory elements will vary according to the particular host.

A Y-box protein-binding immunoglobulin (or fragment thereof) as described herein may contain a detectable moiety or label such as an enzyme, cytotoxic agent or other reporter molecule, including a dye, radionuclide, luminescent group, fluorescent group, or biotin, or the like. The Y-box protein-specific immunoglobulin or fragment thereof may be radiolabeled for diagnostic or therapeutic applications. Techniques for radiolabeling of antibodies are known in the art. See, e.g., Adams 1998 In Vivo 12:11-21; Hiltunen 1993 Acta Oncol. 32:831-9. Therapeutic applications are described in greater detail below and may include use of the Y-box protein-binding antibody (or fragment thereof) in conjunction with other therapeutic agents. The antibody or fragment may also be conjugated to a cytotoxic agent as known in the art and provided herein, for example, a toxin, such as a ribosome-inactivating protein, a chemotherapeutic agent, an anti-mitotic agent, an antibiotic or the like.

The invention also contemplates the generation of anti-idiotype antibodies that recognize an antibody (or antigen-binding fragment thereof) that specifically binds to Y-box protein as provided herein, or a variant or fragment thereof. Anti-idiotype antibodies may be generated as polyclonal antibodies or as monoclonal antibodies by the methods described herein, using an anti-Y-box protein antibody (or antigen-binding fragment thereof) as immunogen. Anti-idiotype antibodies or fragments thereof may also be generated by any of the recombinant genetic engineering methods described above, or by phage display selection. An anti-idiotype antibody may react with the antigen binding site of the anti-Y-box protein antibody such that binding of the anti-Y-box protein antibody to a Y-box protein polypeptide is competitively inhibited. Alternatively, an anti-idiotype antibody as provided herein may not competitively inhibit binding of an anti-Y-box protein antibody to a Y-box protein polypeptide.

As provided herein and according to methodologies well known in the art, polyclonal and monoclonal antibodies may be used for the affinity isolation of Y-box protein polypeptides. See, e.g., Hermanson et al., Immobilized Affinity Ligand Techniques, Academic Press, Inc. New York, 1992. Briefly, an antibody (or antigen-binding fragment thereof) may be immobilized on a solid support material, which is then contacted with a sample comprising the polypeptide of interest (e.g., a Y-box protein). Following separation from the remainder of the sample, the polypeptide is then released from the immobilized antibody.

Modulating agents may be used to modulate, modify or otherwise alter (e.g., increase or decrease with statistical significance) cellular responses such as cell proliferation, differentiation and survival, in a variety of contexts, both in vivo and in vitro. In general, to so modulate (e.g., increase or decrease in a statistically significant manner) such a response, a cell is contacted with an agent that modulates PTP1B expression levels, under conditions and for a time sufficient to permit modulation of PTP1B activity. Agents that modulate a cellular response may function in any of a variety of ways. For example, an agent may modulate a pattern of gene expression (i.e., may enhance or inhibit expression of a family of genes or genes that are expressed in a coordinated fashion). A variety of hybridization and amplification techniques are available for evaluating patterns of gene expression. Alternatively, or in addition, an agent may up- or down-regulate one or more components of a PTP1B signal transduction pathway, and/or may effect apoptosis or necrosis of the cell, and/or may modulate the functioning of the cell cycle within the cell. (See, e.g., Ashkenazi et al., 1998 Science, 281:1305; Thornberry et al., 1998 Science 281:1312; Evan et al., 1998 Science 281:1317; Adams et al., 1998 Science 281:1322; and references cited therein.)

Cells treated as described above may exhibit standard characteristics of cells having altered proliferation, differentiation or survival properties. In addition, such cells may (but need not) display alterations in other detectable properties, such as contact inhibition of cell growth, anchorage independent growth or altered intercellular adhesion. Such properties may be readily detected using techniques with which those having ordinary skill in the art will be familiar.

Therapeutic Methods

One or more PTP1B −155/−132 polynucleotides and/or modulating agents and/or Y-box (e.g., YB-1) polypeptides, modulating agents and/or polynucleotides encoding such polypeptides and/or modulating agents may also be used to modulate PTP1B activity in a patient. As used herein, a “patient” may be any mammal, including a human, and may be afflicted with a condition associated with PTP1B activity or may be free of detectable disease. Accordingly, the treatment may be of an existing disease or may be prophylactic. Conditions associated with PTP1B activity are described above and preferably include diabetes and obesity but may also include any disorder associated with cell proliferation, including cancer, graft-versus-host disease (GVHD), autoimmune diseases, allergy or other conditions in which immunosuppression may be involved, metabolic diseases, abnormal cell growth or proliferation and cell cycle abnormalities. Certain such disorders may involve loss of normal MAP-kinase phosphatase activity, leading to uncontrolled cell growth. Therapeutic agents identified according to the present invention can be used to ameliorate such disorders.

For administration to a patient, one or more polypeptides, polynucleotides and/or modulating agents are generally formulated as a pharmaceutical composition. A pharmaceutical composition may be a sterile aqueous or non-aqueous solution, suspension or emulsion, which additionally comprises a physiologically acceptable carrier (i.e., a non-toxic material that does not interfere with the activity of the active ingredient). Such compositions may be in the form of a solid, liquid or gas (aerosol). Alternatively, compositions of the present invention may be formulated as a lyophilizate or compounds may be encapsulated within liposomes using well known technology. Pharmaceutical compositions within the scope of the present invention may also contain other components, which may be biologically active or inactive. Such components include, but are not limited to, buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, stabilizers, dyes, flavoring agents, and suspending agents and/or preservatives.

Any suitable carrier known to those of ordinary skill in the art may be employed in the pharmaceutical compositions of the present invention. Carriers for therapeutic use are well known, and are described, for example, in Remingtons Pharmaceutical Sciences, Mack Publishing Co. (A. R. Gennaro ed. 1985). In general, the type of carrier is selected based on the mode of administration. Pharmaceutical compositions may be formulated for any appropriate manner of administration, including, for example, topical, oral, nasal, intrathecal, rectal, vaginal, sublingual or parenteral administration, including subcutaneous, intravenous, intramuscular, intrasternal, intracavernous, intrameatal or intraurethral injection or infusion. For parenteral administration, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, kaolin, glycerin, starch dextrins, sodium alginate, carboxymethylcellulose, ethyl cellulose, glucose, sucrose and/or magnesium carbonate, may be employed.

A pharmaceutical composition (e.g., for oral administration or delivery by injection) may be in the form of a liquid (e.g., an elixir, syrup, solution, emulsion or suspension). A liquid pharmaceutical composition may include, for example, one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. A parenteral preparation can be enclosed in ampoules, disposable syringes or multiple dose vials made of glass or plastic. The use of physiological saline is preferred, and an injectable pharmaceutical composition is preferably sterile.

The compositions described herein may be formulated for sustained release (i.e., a formulation such as a capsule or sponge that effects a slow release of compound following administration). Such compositions may generally be prepared using well known technology and administered by, for example, oral, rectal or subcutaneous implantation, or by implantation at the desired target site. Sustained-release formulations may contain an agent dispersed in a carrier matrix and/or contained within a reservoir surrounded by a rate controlling membrane. Carriers for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated or prevented.

For pharmaceutical compositions comprising a PTP1B −155/−132 polynucleotide and/or a YB-1 antisense polynucleotide or ribozyme and/or a polynucleotide encoding a polynucleotide or polypeptide modulating agent that impairs (e.g., inhibits by at least 25%, preferably at least 50%, more preferably at least 70%, more preferably at least 85%, more preferably greater than 95%) binding of a Y-box protein to a PTP1B promoter Y-box protein binding site (such that the polypeptide and/or modulating agent is generated in situ), the polynucleotide may be present within any of a variety of delivery systems known to those of ordinary skill in the art, including nucleic acid, and bacterial, viral and mammalian expression systems. Techniques for incorporating DNA into such expression systems are well known to those of ordinary skill in the art. The DNA may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells. In addition to direct in vivo procedures, ex vivo procedures may be used in which cells are removed from a host, modified, and placed into the same or another host animal. It will be evident that one can utilize any of the compositions noted above for introduction of a PTP1B −155/−132 polynucleotide and/or a YB-1 antisense polynucleotide or ribozyme and/or a polynucleotide encoding a polynucleotide or polypeptide modulating agent into tissue cells in an ex vivo context. Protocols for viral, physical and chemical methods of uptake are well known in the art.

Within a pharmaceutical composition, a therapeutic polypeptide, polynucleotide or modulating agent may be linked to any of a variety of compounds. For example, such an agent may be linked to a targeting moiety (e.g., a monoclonal or polyclonal antibody, a protein or a liposome) that facilitates the delivery of the agent to the target site. As used herein, a “targeting moiety” may be any substance (such as a compound or cell) that, when linked to an agent enhances the transport of the agent to a target cell or tissue, thereby increasing the local concentration of the agent. Targeting moieties include antibodies or fragments thereof, receptors, ligands and other molecules that bind to cells of, or in the vicinity of, the target tissue. An antibody targeting agent may be an intact (whole) molecule, a fragment thereof, or a functional equivalent thereof. Examples of antibody fragments are F(ab′)₂, −Fab′, Fab and F[v] fragments, which may be produced by conventional methods or by genetic or protein engineering. Linkage is generally covalent and may be achieved by, for example, direct condensation or other reactions, or by way of bi- or multi-functional linkers. Targeting moieties may be selected based on the cell(s) or tissue(s) toward which the agent is expected to exert a therapeutic benefit.

Pharmaceutical compositions may be administered in a manner appropriate to the PTP1B associated disorder or disease to be treated (or prevented). An appropriate dosage and a suitable duration and frequency of administration will be determined by such factors as the condition of the patient, the type and severity of the patient's disease, the particular form of the active ingredient and the method of administration. In general, an appropriate dosage and treatment regimen provides the agent(s) in an effective amount sufficient to provide therapeutic and/or prophylactic benefit (e.g. an improved clinical outcome, such as more frequent complete or partial remissions, or longer disease-free and/or overall survival). For prophylactic use, a dose should be sufficient to prevent, delay the onset of or diminish the severity of a disease associated with cell proliferation.

Optimal dosages may generally be determined using experimental models and/or clinical trials. In general, the amount of polypeptide present in a dose, or produced in situ by DNA present in a dose, ranges from about 0.01 μg to about 100 μg per kg of host, typically from about 0.1 μg to about 10 μg. The use of the minimum dosage that is sufficient to provide effective therapy is usually preferred. Patients may generally be monitored for therapeutic or prophylactic effectiveness using assays suitable for the condition being treated or prevented, which will be familiar to those having ordinary skill in the art. Suitable dose sizes will vary with the size of the patient, but will typically range from about 10 mL to about 500 mL for 10-60 kg animal.

The following Examples are offered for the purpose of illustrating the present invention and are not to be construed to limit the scope of this invention.

### EXAMPLES

### Example 1

### Identification of the Transcriptional Enhancing Element for PTP1B Expression (TEP)

This Example illustrates identification of the promoter sequence that comprises the transcriptional enhancing element for PTP1B expression (TEP).

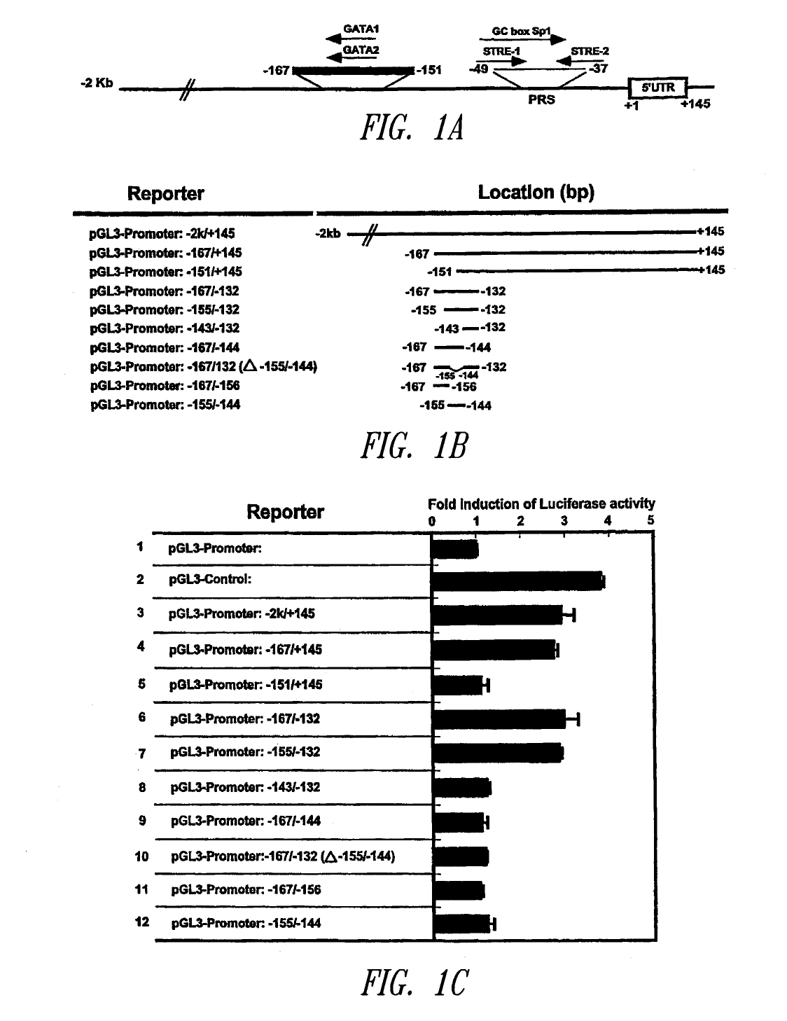

Previous studies demonstrated that p210 Bcr-Abl upregulates PTP1B expression (LaMontagne et al., Mol. Cell. Biol. 18:2965-75 (1998); LaMontagne et al., Proc. Natl. Acad. Sci. USA 95:14094-99 (1998)). A p210 Bcr-Abl-responsive sequence (PRS) is located 49 to −37 base pairs upstream from the transcriptional start site for human PTP1B and contains a sequence that displays features of a stress response element (STRE) (Fukada et al., J. Biol. Chem. 276:25512-19 (2001)) (FIG. 1A). A second motif within the PTP1B promoter, located at −167 to −151 base pairs from the transcription start site, possesses features for a site of interaction with GATA-binding proteins (id.) (FIG. 1A). Disruption of this site inhibited promoter activity, but promoter activity responsive to p210 Bcr-Abl was maintained (id.).

To more precisely define the transcription enhancing element, a series of sequences described in FIG. 1B were created and each was inserted into a MluI/XhoI restriction enzyme site in a reporter plasmid containing the SV40 minimal promoter and the firefly luciferase gene (pGL3-Promoter, Promega, Madison, Wis.) (FIG. 1B). The following double-stranded DNAs corresponding to nucleotide sequences at different locations within the PTP1B promoter and gene were synthesized and used for plasmid construction.

−167/−132 sense: 5′-CGCGTACGCGCGCTATTAGATATCTCGCGGTGCTGGGGCCAC-3′ (SEQ ID NO: 6)

−162/−132 antisense: 5′-TCGAGTGGCCCCAGCACCGCGAGATATCTAATAGCGCGCGTA-3′ (SEQ ID NO: 7)

−167/−144 sense: 5′-CGCGTACGCGCGCTATTAGATATCTCGCGC-3′ (SEQ ID NO: 8)

−167/−144 antisense: 5′-TCGAGCGCGAGATATCTAATAGCGCGCGTA-3′ (SEQ ID NO: 9)

−167/−156 sense: 5′-CGCGTACGCGCGCTATT-3′ (SEQ ID NO: 10)

−167/−156 antisense: 5′-TCGAGAATAGCGCGCGTA-3′ (SEQ ID NO: 11)

−155/−132 sense: 5′-CGCGTAGATATCTCGCGGTGCTGGGGCCAC-3′ (SEQ ID NO: 12)

−155/−132 antisense: 5′-TCGAGTGGCCCCAGCACCGCGAGATATCTA-3′ (SEQ ID NO: 13)

−155/−144 sense: 5′-CGCGTAGATATCTCGCGC-3′ (SEQ ID NO: 14)

−155/−144 antisense: 5′-TCGAGCGCGAGATATCTA-3′ (SEQ ID NO: 15)

−143/−132 sense: 5′-CGCGTGTGCTGGGGCCAC-3′ (SEQ ID NO: 16)

−143/−132 antisense: 5′-TCGAGTGGCCCCAGCACCGCGAGATATCTA-3′ (SEQ ID NO: 17)

−167/−132 (Δ−155/−144) sense:

5′-CGCGTAACGCGCGCTATTGTGCTGGGGCCAC-3′ (SEQ ID NO: 18)

−167/−132 (Δ−155/−144) antisense: 5′-TCGAGTGGCCCCAGCACA-3′ (SEQ ID NO: 19)

A series of 5′-deletion mutants of the PTP1B 5′ flanking region of the human PTP1B gene were made by PCR using −2 k/+145 as a template (see Fukada et al., supra). The oligonucleotides used as 5′ primers for making the deletion mutants were as follows.

−2 k/+145: 5′-GGTACCGAGCTCTTACGCGT-3′(SEQ ID NO: 20)

−167/+145: 5′-ACTATAGGGCACGCGTACGCGCGCTATTAGATATCT-3′(SEQ ID NO: 21)

−151/+145: 5′-ACTATAGGGCACGCGTATCTCGCGGTGCTGGGGCC-3′(SEQ ID NO: 22)

The 3′ primer sequence for making the deletion mutants was 5′-CCCCTCGAGGACGGGCCAGGGCGGCTGCTGCGCCTCCTT-3′ (SEQ ID NO: 23). The amplified PCR products were digested by restriction enzymes, MluI and XhoI, and the digested products were inserted into the pGL3-Basic promoter plasmid (Promega). Insertion of the correct PCR product was confirmed by sequencing.

Parental Rat1 fibroblasts, cultured in Dulbecco's modified Eagle's medium (DMEM) supplemented with 10% fetal bovine serum, were transfected using LIPOFECTAMINE Reagent (Invitrogen Life Technologies, Carlsbad, Calif.). Typically, for a reporter assay, 1 μg of the reporter plasmid was used for expression of firefly luciferase, and 1 μg of pRL-TK (Promega), which is an expression vector containing cDNA encoding Renilla luciferase, was used as an internal control for transfection efficiency. Cells were incubated with DNA-lipid complex for 24 hours, washed with phosphate buffered saline, and luciferase activity was assayed using the Dual-Luciferase Reporter Assay System (Promega) (FIG. 1C).

Values were normalized for transfection efficiency and represent the mean +/−S.D. of three separate experiments. These data suggest that the sequence between −155 and −132 base pairs is a minimal enhancing element and likely plays a role in regulation of PTP1B gene expression.

### Example 2

### Identification of the Binding Protein to the Transcriptional Enhancing Element for PTP1B Expression (B-TEP)

This Example illustrates identification of a protein that binds to TEP (B-TEP). Nuclear extracts were probed with radiolabeled oligonucleotides in an electrophoretic mobility shift assay (EMSA). Proteins were isolated from the nuclear extracts by chromatography and characterized.

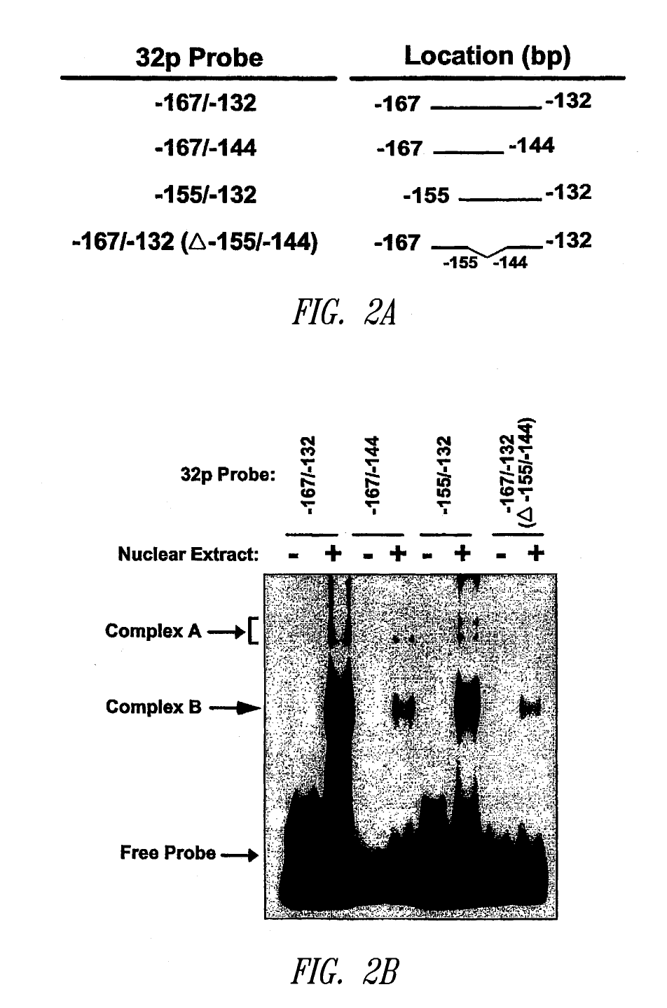

An electrophoretic mobility shift assay was performed to identify TEP by formation of DNA-protein complexes. Nuclear extracts were prepared from Rat1 cells using a nuclear extraction buffer (20 mM HEPES pH 7.9, 20 mM NaF, 1 mM Na₃VO₄, 1 mM Na₄P₂O₇, 0.125 μM okadaic acid, 1 mM EDTA, 1 mM EGTA, 1 mM DTT, 0.5 mM PMSF, 1 μg/ml leupeptin, 1 μg/ml aprotinin, 1 μg/ml pepstatin, 0.2% NP-40, 5% glycerol, 420 mM NaCl) according to the method of Sadowski and Gilman (Sadowski et al., Nature 362:79-83 (1993)). Double-stranded oligonucleotides were end-labeled using a 5′ end labelling kit (Pharmacia, Peapack, N.J.) and purified by ProbeQuant™ G-50 Micro columns (Pharmacia). The following double-stranded DNA probes were used (see FIG. 2A).

−167/−132 sense: 5′-ACGCGCGCTATTAGATATCTCGCGGTGCTGGGGCCA-3′(SEQ ID NO: 24)

−167/−132 antisense: 5′-TGGCCCC AGCACCGCGAGATATCTAATAGCGCGCGT-3′(SEQ ID NO: 25)

−167/−144 sense: 5′-ACGCGCGCTATTAGATATCTCGCG-3′(SEQ ID NO: 26)

−167/−144 antisense: 5′-CGCGAGATATCTAATAGCGCG CGT-3′(SEQ ID NO:27)

−155/−132 sense: 5′-AGATATCTCGCGGTGCTGGGGCCA-3′(SEQ ID NO: 28)

−155/−132 antisense: 5′-TGGCCCCAGCACCGCGAGATATCT-3′(SEQ ID NO:29)

−167/−132 (Δ−155/−144) sense: 5′-ACGCGCGCTATTGTGCTGGGGCCA-3′(SEQ ID NO: 30)

−167/−132 (Δ−155/−144) antisense: 5′-TGGCCCCAGCACAATAGCGCGCGT-3′(SEQ ID NO: 31)

Each probe (1×10⁵ cpm) was incubated at 25° C. for 20 minutes with 5 μg of nuclear extract in binding buffer (10 mM HEPES-KOH pH 7.8, 50 mM KCl, 1 mM EDTA, 5 mM MgCl₂, 10% glycerol, 5 mM DTT, 0.7 mM PMSF, 2 μg/ml aprotnin, 2 μg/ml pepstatin, 2 μg/ml leupeptin, 1 mM Na₃VO₄, and 0.4 μg/ml poly dI: dC). The extracts were then electrophoretically separated. Specific binding between proteins and the DNA probe was quantified by drying the EMSA gel and scanning the dried gel with a Fuji BASS 2000 phosphoimager to detect radioactivity in the DNA-protein complex. Results are presented in FIG. 2B. In cases in which specific activity is determined, the specific activity (photostimulated luminescence (PSL)/mm²/μg) was obtained by dividing the PSL of the DNA-protein complex in a defined surface on the gel (PSL/mm²) by the amount of protein (μg) used for EMSA.

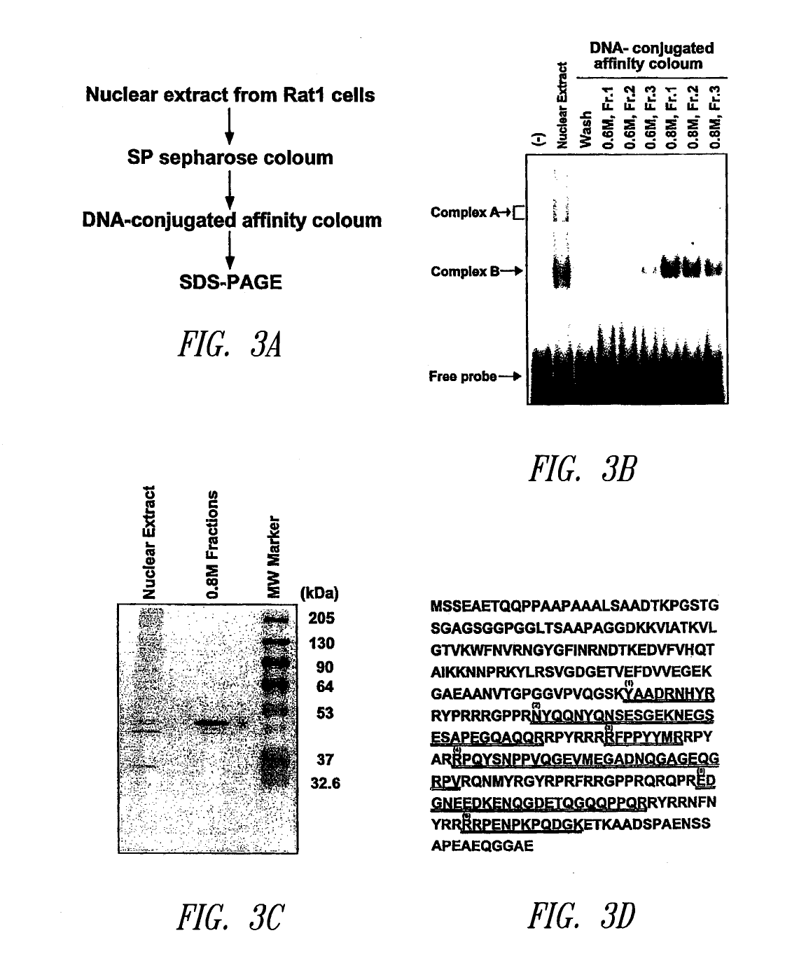

To purify proteins within the TEP sequence complexes, nuclear extracts were subjected to SP-Sepharose chromatography followed by DNA-affinity chromatography (FIG. 3A). Nuclear extracts (approximately 50 mg) were obtained from Rat1 cells as described above. The extracts were dialyzed against starting buffer (0.1 M NaCl in 20 mM HEPES pH 7.9, 20 mM NaF, 1 mM Na₃VO₄, 1 mM Na₄P₂O₇, 0.125 μM okadaic acid, 1 mM EDTA, 1 mM EGTA, 1 mM DTT, 0.5 mM PMSF, 1 μg/ml leupeptin, 1 μg/ml aprotinin, 1 μg/ml pepstatin, 0.2% NP-40, 5% glycerol) and then applied to SP Sepharose (20 ml) (Pharmacia Biotech, Piscataway, N.J.) equilibrated with the starting buffer. Proteins were eluted in a stepwise manner with 0.2, 0.3, 0.4, 0.6, and 0.8 M NaCl containing nuclear extraction buffer. Each fraction (5 μl aliquots) was assayed by EMSA using ³²P-labelled probes as described above. Fractions containing Complex B (detected by the ³²P-labeled −167/−132 probe, see FIG. 2B) were dialyzed to 0.2 M NaCl containing starting buffer and then applied to a double-stranded DNA-conjugated affinity column. The sequence of the DNA used for affinity chromatography corresponded to nucleotides −167 to −132 from the transcription start site as follows:

Sense: biotin-5′-ACGCGCGCTATTAGATATCTCGCGGTGCTGGGGCCA-3′ (SEQ ID NO:32)

Anti-sense: biotin-5′-TGGCCCCAGCACCGCGAGATATCTAATAGCGCGCGT-3′ (SEQ ID NO:33).

The DNA was biotinylated according to standard methods. Biotinylated DNA was conjugated to 2 ml of streptavidin-sepharose high performance (Pharmacia) according to manufacturer's instructions. Bound proteins were eluted in a stepwise manner with 0.2, 0.3, 0.4, 0.6, 0.8 M NaCl containing starting buffer. Each fraction (5 μl aliquots) was assayed by EMSA to detect Complex B using the 32P-labeled −167/−132 probe (FIG. 3B). The active fractions (0.8 M NaCl fractions 1-3, FIG. 3B) were concentrated and desalted using Ultrafree-4 Centrifugal Filter Units (Millipore, Bedford, Mass.) with a 5,000 molecular weight cutoff. The concentration fraction (2 μg) and a sample of the nuclear extract (2 μg protein) were further separated by SDS-PAGE and visualized using Brilliant Blue G-Colloidal Concentrate (Sigma, St. Louis, Mo.) (FIG. 3C). The major band (indicated by a * in FIG. 3C) corresponded to a protein of approximately 47 kDal termed B-TEP. The band was sliced from the gel for sequence analysis performed by tandem mass spectrometry (Cold Spring Harbor Laboratory Protein Chemistry Shared Source). The sequence of six peptides was determined and aligned with known protein sequences using methods known in the art. The sequences of the peptides (double-underlined in FIG. 3D) were identical to portions of the amino acid sequence of rat Y box binding protein YB-1 (FIG. 3D).

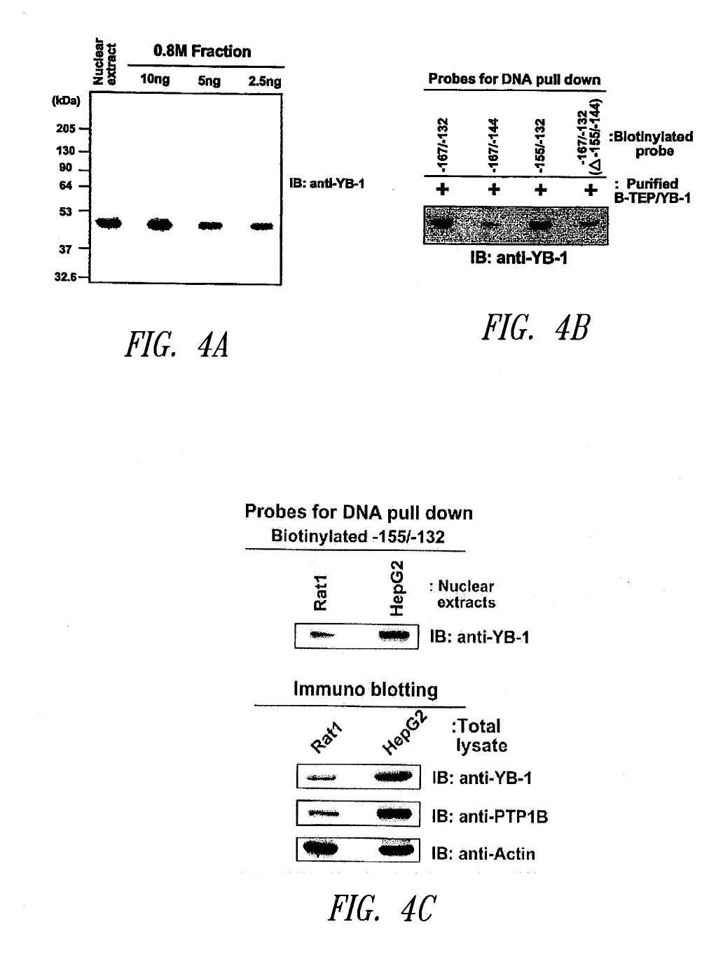

Immunoblot analysis of nuclear extracts and of isolated B-TEP was performed according to standard procedures. Nuclear extracts (20 μg) and purified B-TEP (2.5, 5, and 10 ng per sample) were resolved by SDS-PAGE and transferred to a PVDF-membrane (Millipore). The membrane was immunoblotted with an anti-YB-1 antibody (gift from Dr. DiCorleto) (Stenina et al., J. Clin. Invest. 106:579-87 (2000)) and specific binding was detected by ECL (Pharmacia) (FIG. 4A) further confirming the identity of B-TEP as YB-1.

A DNA pull-down analysis was performed to determine the amount of complex formed between purified YB-1 protein and the PTP1B enhancing sequence. The assay was performed essentially as described (Fukada et al., supra). Purified YB-1 (B-TEP) was incubated with a biotinylated double-stranded DNA probe (200 pmole) and with streptavidin-agarose (20 μl) for 6 hours at 4° C. The probes used were −167/−132, −167/−144, −155/−132, and −167/−132 (Δ−155/−144) (see sequences above). The protein-DNA complexes were subjected to SDS-PAGE, immunoblotted with anti-YB-1, and visualized by ECL (FIG. 4B).

A DNA pull-down analysis was also performed to determine the amount of complex formed between protein B-TEP (hereinafter referred to as YB-1) and the PTP1B enhancing sequence in a second cell line, HepG2 human hepatocellular carcinoma (American Type Culture Collection (ATCC), Manasass, Va.). Nuclear extracts from Rat1 and Hep2 cells were prepared as described above, and the levels of YB-1 and PTP1B were compared. Nuclear extracts from each cell line were incubated with a biotinylated double-stranded DNA probe, −155/−132, which has enhancer activity, followed by an incubation with streptavidin-Sepharose for 6 hours at 4° C. The protein-DNA complexes were subjected to SDS-PAGE, followed by immunoblotting with anti-YB-1 antibody. The immunoblot is shown in FIG. 4C, upper panel.

In addition, total cell lysates (20 μg) of Rat1 and HepG2 cells were each immunoblotted with antibodies specific for YB-1, PTP1B, or actin. Total cell lysates were prepared by suspending cells in 1 ml of lysis buffer (1% NP-40, 20 mM Tris-HCl pH 7.4, 150 mM NaCl, 5 μg/ml aprotinin, 0.1 mM PMSF, 1 mM Na₃VO₄). The immunoblot is shown in FIG. 4C, lower panel. The results indicate that the levels of YB-1 retained by the PTP1B enhancer probe in the nuclear extracts and of YB-1 in total cell lysates were greater in the HepG2 cells than in the Rat1 cells. The level of PTP1B also appeared greater in HepG2 cells compared with Rat1 cells (FIG. 4C, lower panel).

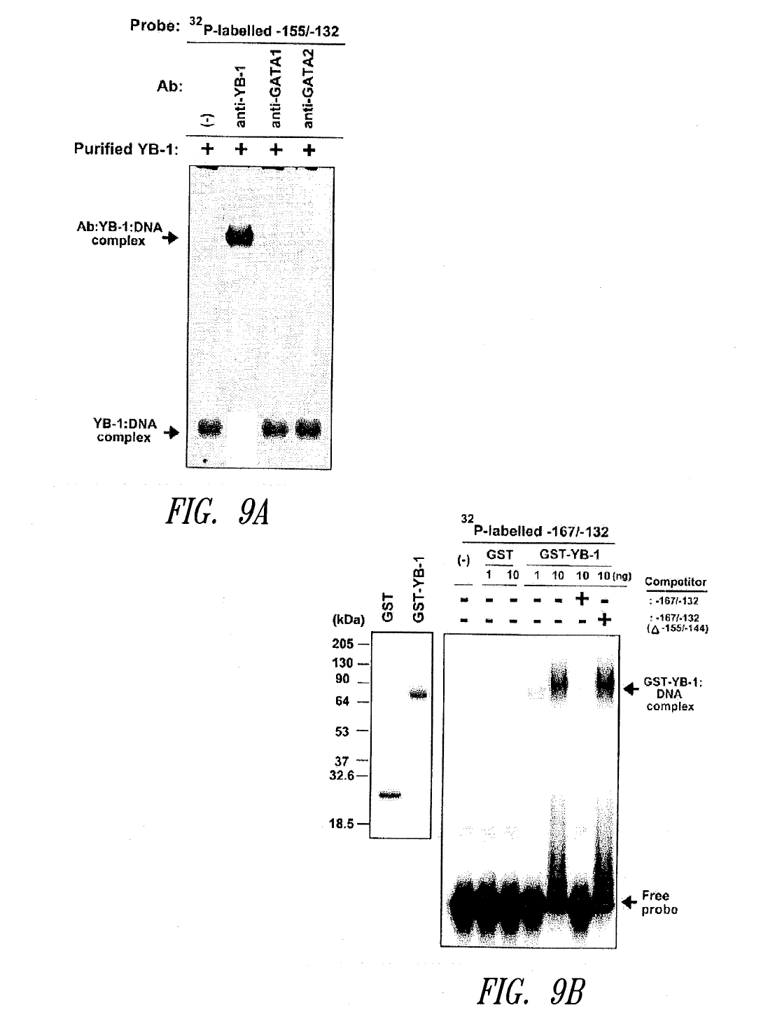

To further examine the interaction of YB-1 with the PTP1B enhancer, an EMSA was performed using purified YB-1 (10 ng) and the double-stranded DNA ³²P-labelled −155/−132 probe (see methods described above). A YB-1/DNA probe complex was detected by incubating the complex with the anti-YB-1 antibody (see above). Included as controls were anti-GATA1 (sc266X) and anti-GATA2 (sc-9008X) antibodies (Santa Cruz Biotechnology, Inc., Santa Cruz, Calif.). Purified YB-1 was incubated without antibody or separately with each antibody for 1 hour at 4° C., followed by addition of the DNA probe for 20 minutes at 25° C. The results showing YB-1 antibody/super-shifted YB-1:DNA complexes are presented in FIG. 9A.

Complex formation of YB-1 with the PTP1B enhancer was confirmed using purified YB-1 from a different source. Expression plasmids for GST and for GST-YB-1 (gift from Dr. K. Kohno) (see Izumi et al., Nucleic Acids Res. 29:1200-07 (2001)) were each separately transfected into E. coli BL21 cells. Recombinant proteins were expressed and purified (see Frangioni and Neel, Anal. Biochem. 210:179-87 (1993)) and analyzed by SDS-PAGE as shown in FIG. 9B (left panel). The interaction between GST-tagged YB-1 and the PTP1B enhancer probe was analyzed by EMSA. Purified GST or GST-YB-1 proteins (1 or 10 ng) were combined with double-stranded DNA ³²P-labelled probe −167/−132 (see above) in the absence or presence of unlabeled double-stranded DNA probe −167/−132 or the deletion mutant probe −167/−132 (Δ−155/−144) (see above), which were tested as competitors of complex formation. The results are presented in FIG. 9B (right panel).

### Example 3

### Regulation of PTP1B Expression In Vivo by YB-1

This Example illustrates the effects of antisense oligonucleotides specific for YB-1 nucleotide sequences on expression of YB-1 and PTP1B and on formation of a complex between YB-1 and the PTP1B enhancing sequence.

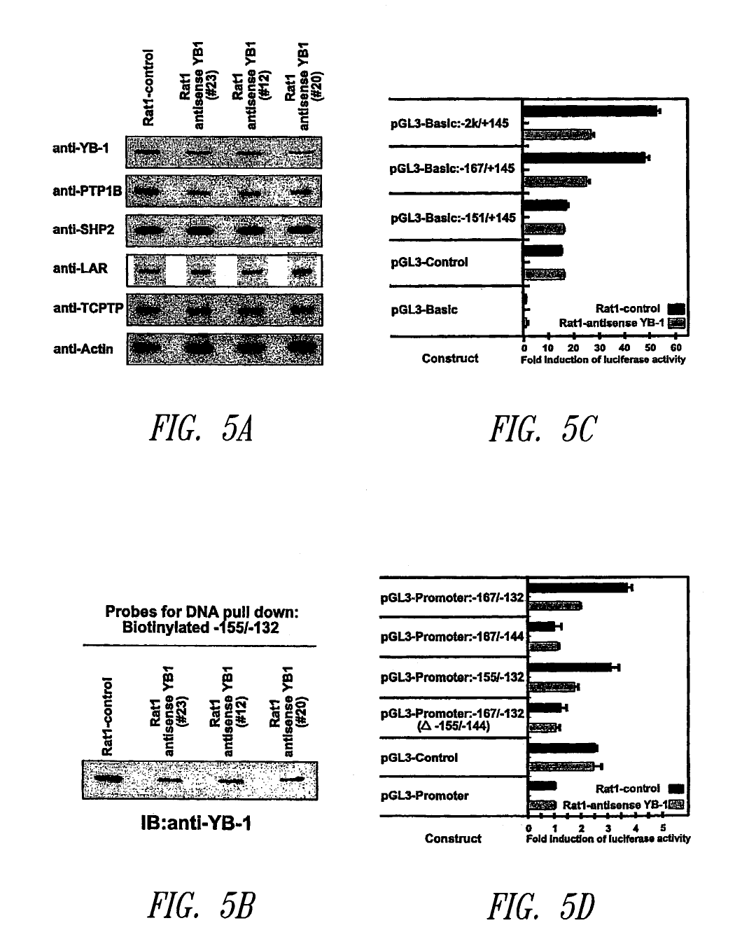

Antisense expression constructs (AS 0.7 YB-1) (clones 12, 20, and 23) (Ohga et al., J. Biol. Chem. 273:5997-6000 (1998)) containing antisense sequences complementary to the coding sequence of rat YB-1 were stably transfected into Rat1 cells cultured in DMEM supplemented with 10% FBS in the presence of G418. Rat1 fibroblasts expressing the antisense YB-1 mRNA were cultured in DMEM supplemented with 10% fetal bovine serum in the presence of G418 (Gibco BRL, Carlsbad, Calif.). Total cell lysates were prepared as described in Example 2, and aliquots (20 μg) were applied to SDS polyacrylamide gels. After transfer of the separated proteins to PVDF membranes (see Example 2), the membranes were immunoblotted with an anti-YB-1 antibody (see Example 2), anti-PTP1B antibody FG6 (LaMontagne et al., Mol. Cell. Biol. 18:2965-75 (1998)); anti-SHP-2 (sc-280, Santa Cruz Biotechnology, Santa Cruz, Calif.); anti-LAR (sc-1119, Santa Cruz Biotechnology); anti-TCPTP 1910 (Lorenzen et al., J. Cell. Biol. 131:631-43 (1995)); or anti-actin (sc-8432, Santa Cruz Biotechnology) antibodies (FIG. 5A).

The amount of complex formed between YB-1 protein and the PTP1B enhancing sequence was determined by a DNA-pull down assay (see Example 2). Nuclear extracts from Rat1 control or from Rat1 antisense-YB-1 expression constructs (clones 12, 20, and 23) were prepared as described in Example 1. Nuclear extracts (100 μg) were combined with the biotinylated −155/−132 probe (200 pmole) (see Example 2) and 20 μl streptavidin-sepharose high performance (Pharmacia) at 4° C. for 6 hours in starting buffer (see Example 2) (1 ml final volume). The protein-DNA complexes were resolved by SDS-PAGE, immunoblotted with anti-YB-1, and visualized by ECL (FIG. 5B).

Transcription assays using the luciferase reporter were performed to evaluate the effect of YB-1 expression levels on intact PTP1B promoter activity (see Example 2). A series of 5′-deletion mutants of the PTP1B 5′ flanking region of the human PTP1B gene were made by PCR using −2 k/+145 as a template (see Fukada et al., supra) as described in Example 1. The oligonucleotides used as 5′ primers for making the deletion mutants were −2 k/+145: (SEQ ID NO: 20); −167/+145: (SEQ ID NO: 21); and −151/+145: (SEQ ID NO: 22). The 3′ primer sequence for making the deletion mutants was 5′-CCCCTCGAGGACGGGCCAGGGCGGCTGCTGCGCCTCCTT-3′ (SEQ ID NO:34). The amplified PCR products were digested by restriction enzymes, MluI and XhoI, and the digested products were inserted into the pGL3-Basic promoter plasmid (Promega). Insertion of the correct PCR product was confirmed by sequencing.

Control Rat1 cells and Rat1 cells stably transfected with a YB-1 antisense construct (clone 20) were transiently transfected with 1 μg of a luciferase construct containing segments of the human PTP1B promoter (see Example 1 for transfection method). Controls included plasmid that contained the SV40 promoter and enhancer sequence (pGL3-control), empty vector that did not contain a eukaryotic promoter or enhancer sequences (pGL3-Basic), and 1 μg of pRL-TK to normalize transfection efficiency. Cells were harvested, lysed, and assayed for luciferase activity (FIG. 5C). Values were normalized for transfection efficiency and represent the mean +/−S.D. of three separate experiments.

Transcription assays were also performed to determine the effect of YB-1 expression levels on TEP-driven transcription enhancing activity. Experiments were performed essentially as described above except that the luciferase constructs contained segments of the human PTP1B transcriptional enhancing elements, −167/−132, −167/−144, −155/−132, and −167/−132 (Δ−155/−144) (see sequences in Example 2), and the SV40 promoter. Control plasmids contained SV40 promoter and enhancer sequence (pGL3-control), empty vector contained the SV40 promoter and no PTP1B sequences (pGL3-Promoter), and 1 μg of pRL-TK to normalize transfection efficiency. Luciferase activity was measured as described above. (See FIG. 5D).

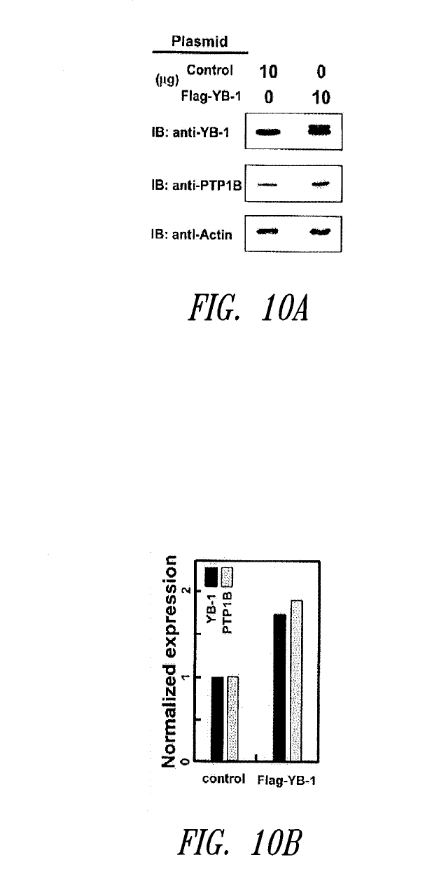

The reciprocal experiment was performed to examine the effect of YB-1 overexpression (instead of antisense inhibition of YB-1 expression) on the level of PTP1B. Rat1 cells were transiently transfected with 10 μg of expression plasmid containing either FLAG®-tagged YB-1 (FLAG®-YB-1) (gift from Drs. M. Kuwano and T. Uchiumi) or pCMV-tag empty vector control. (FLAG® sequence: Asp-Tyr-Lys-Asp-Asp-Asp-Asp-Lys (SEQ ID NO:35)) (Sigma Aldrich, St. Louis, Mo.). The plasmids were prepared according to standard molecular biology procedures. Total Rat1 cell lysates (20 μg) were prepared as described in Example 2 and were separated by SDS-PAGE and then immunoblotted with antibodies specific for YB-1, PTP1B, and actin. The immunoblots are shown in FIG. 10A, and densitometric analyses of the gel images are presented in FIG. 10B.

The interaction between YB-1 and PTP1B was explored further by measuring the levels of YB-1 and PTP1B mRNA expression in various cell lines. The expression pattern of YB-1 and PTP1B was analyzed using the Human Tumor Multiple Tissue cDNA (MTC™) Panel (BD Biosciences Clontech, Palo Alto, Calif.). The sources of cDNA in the panel were the following cancer cell lines: 293 (transformed primary embryonal kidney); SK-OV-3 (malignant ascites fluid from ovary adenocarcinoma); Saos-2 (osteosarcoma); A-431 (epidermoid carcinoma); DU 145 (prostate carcinoma metastatic to brain); H1299 (non-small cell lung carcinoma); Hela (cervical epitheloid carcinoma); and MCF7 (breast adenocarcinoma). PCR was performed according to the manufacturer's protocol, using the following primers.

For YB-1: 5′-TTGGGAACAGTAAAATGGTTCAAT-3′ (sense) (SEQ ID NO:36) 5′-CTGCTTCTGTCTCTTTGCCATCTT-3′ (antisense) (SEQ ID NO:37)

For PTP1B: 5′-ATGGAGATGGAAAAGGAGTTCGAG-3′ (sense) (SEQ ID NO:38) 5′-GATATACTCATTATCTTCTTGATG-3′ (antisense) (SEQ ID NO:39)

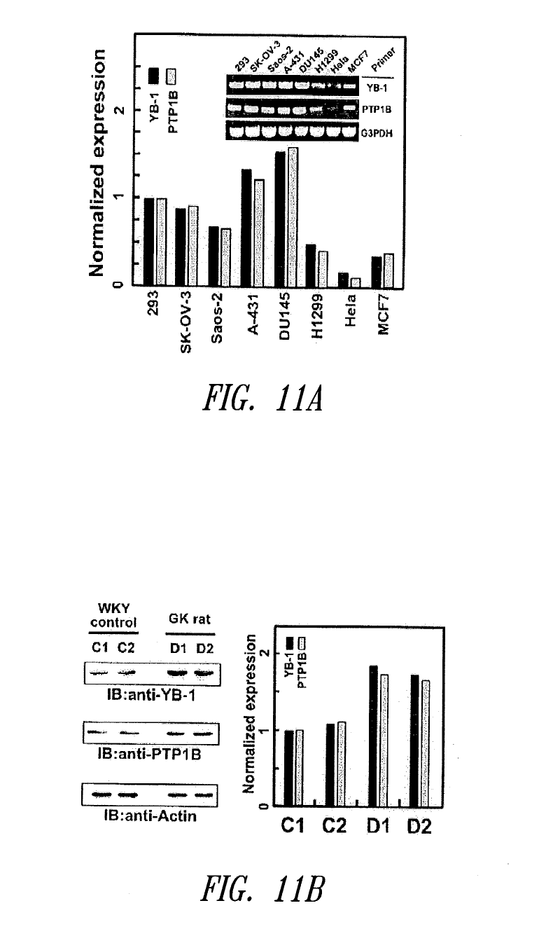

Expression of G3PDH using cDNA primers supplied by the manufacturer (BD Biosciences Clontech) was measured as a control. The amplified products were separated by agarose gel electrophoresis. For each cell type, the level of YB-1 and PTP1B cDNA was quantified by densitometry of the agarose gel image and was standardized relative to the level of the amplified G3PDH control cDNA. In FIG. 11A, densitometric analyses of the gel (inset) are shown. For comparison, the ratio of the level of YB-1 cDNA and PTP1B cDNA to the level of G3PDH cDNA in 293 cells was arbitrarily assigned a value of 1, and the data presented for the other cell lines are relative to that value (see FIG. 11A).

Expression of PTP1B and YB-1 polypeptides was also evaluated in an animal model of diabetes. Skeletal muscle extracts were prepared from non-obese, insulin-resistant type II diabetic Goto-Kakazaki (GK) rats and from non-diabetic, control WKY rats, which were comparable in age and weight (see Dadke et al., Biochem. Biophys. Res. Commun. 274:583-89 (2000)). The rats were provided by Dr. Najma Begum (Diabetes Research Laboratory, Winthrop Hospital and SUNY Stony Brook, Mineola, N.Y.) (see also Dadke et al., supra). The GK rats (D1 and D2) displayed typical characteristics of type II diabetes, such as high blood sugar, and their average body weight was 20 g heavier than the WKY controls (C1 and C2). Skeletal muscle samples (˜2 g) were homogenized in 50 mM Tris-HCl pH 8.0, 137 mM NaCl, 10% glycerol, 1% NP-40, 1 mM PMSF, 1 μg/ml aprotinin, 1 μg/ml leupeptin, 0.5 mM EDTA). The homogenates were then clarified by centrifugation. The tissue homogenates (20 μg) were subjected to SDS-PAGE followed by immunoblotting with antibodies to YB-1, PTP1B, or to actin (control). The immunoblots are presented in FIG. 11B (left panel). The graph shown in FIG. 11B, right panel, represents densitometric analyses of the gel images to illustrate the ratio of expression of YB-1 and PTP1B compared to the actin control.

### Example 4

### Effect of Decreased Expression of YB-1 on Insulin and Cytokine Sensitivity

This Example illustrates the effect of decreased expression of YB-1 in cells stimulated with insulin. This Example also illustrates the effect of decreased expression of YB-1 on cytokine-mediated signaling.

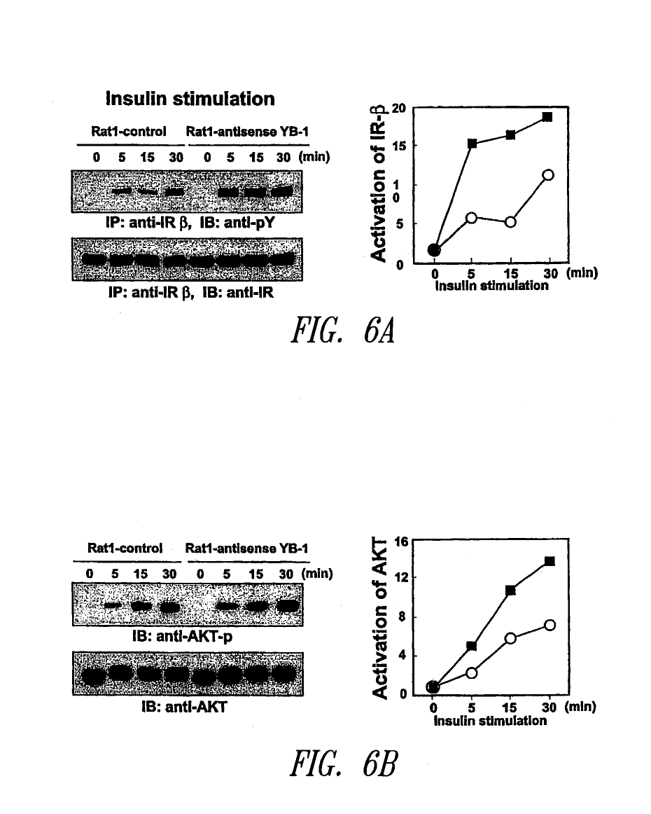

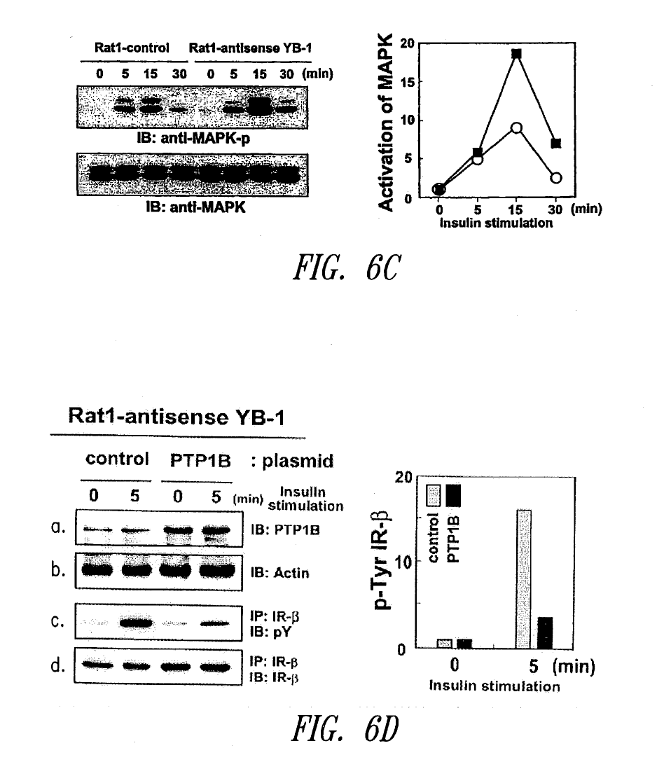

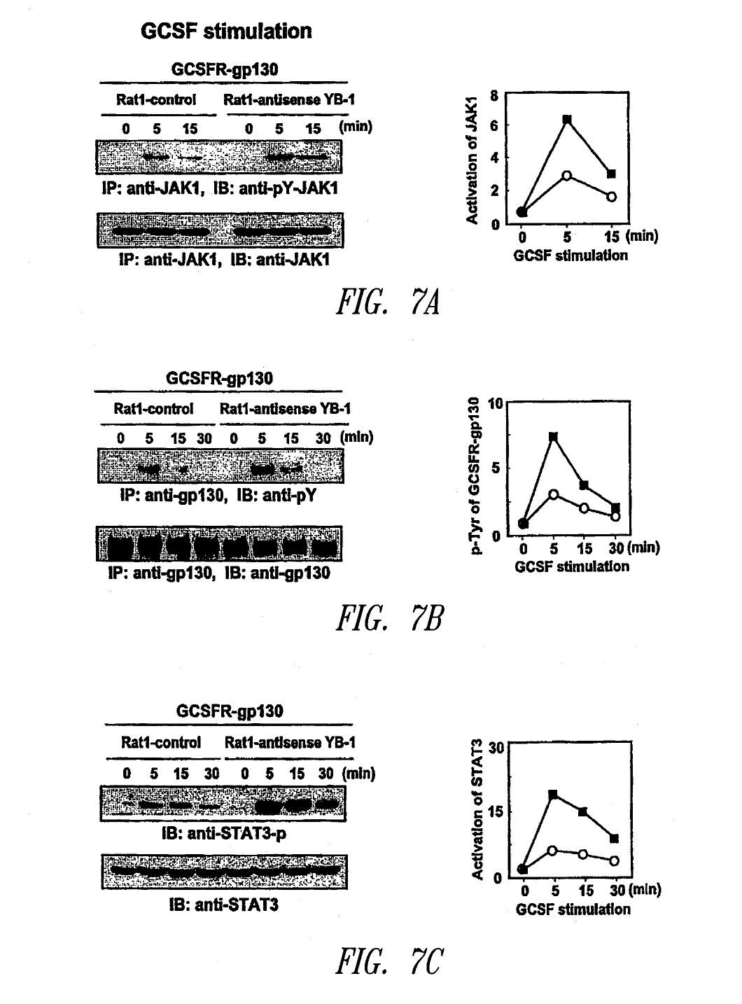

Control Rat1 cells (Rat1-control) and Rat1 cells stably transfected (see Example 1) with an YB-1 antisense construct (clone number 20) (Rat1-antisense YB-1) were starved of serum for 6 hours and then stimulated with insulin (500 nM) (Calbiochem, San Diego, Calif.) for 5, 15, or 30 minutes. Immunoprecipitation of the insulin receptor β (IR β) was performed as follows. Cells were suspended in 1 ml of lysis buffer (1% NP-40, 20 mM Tris-HCl pH 7.4, 150 mM NaCl, 5 μg/ml aprotinin, 0.1 mM PMSF, 1 mM Na₃VO₄). Lysates were cleared by centrifugation and mixed with 20 μl of protein A-sepharose or protein G-sepharose (Pharmacia), and 1 μg of anti-IR β (BD Transduction Laboratories, Lexington, Ky.). The immunoprecipitates were eluted from the sepharose beads with SDS-PAGE loading buffer, separated by SDS-PAGE, and transferred to a PVDF membrane. The membranes were immunoblotted with either anti-phosphotyrosine antibodies (anti-pY) (sc-508, Santa Cruz Biotechnology) and G104 (Garton et al., Oncogene 15:877-85 (1997)) or the anti-IR β antibody (FIG. 6A).

To determine the effects of stimulation of insulin on molecules that are downstream of insulin signaling, total cell lysates (20 μg per lane) were submitted to SDS-PAGE and immunoblotted as described above. After transfer, the PVDF membranes were blotted with an anti-phospho-Akt or an anti-Akt antibody (Cell Signaling Technology, Beverly, Mass.) (FIG. 6B). Immunoblot analysis was also performed with an anti-phospho-MAPK(Erk1/2) or an anti-MAPK(Erk1/2) antibody (Cell Signaling Technology) (FIG. 6C).

The increased phosphorylation of the insulin receptor (IR), which was observed in Rat1 cells that were transfected with YB-1 antisense RNA, was counteracted by forced re-expression of PTP1B. Rat1 clone number 20 cells (Rat1-antisense YB-1) (see above) were transiently transfected with expression plasmids (10 μg each) for either PTP1B (Flint et al., Proc. Natl. Acad. Sci. USA 94:1680-85 (1997)) or pMT2 empty vector (control) according to standard molecular biology procedures and procedures described herein. The transfected cells were starved of serum as described above and then stimulated with insulin (500 nM) for five minutes. Total cell lysate (20 μg) was subjected to SDS-PAGE followed by immunoblotting with antibodies specific for PTP1B and actin (control). In addition, the IR β-subunit was immunoprecipitated as described above and immunoblotted with an antibody specific for anti-phosphotyrosine and with an antibody specific for the anti-IR β-subunit. The results of these experiments are presented in FIG. 6D. The graph (FIG. 6D, right panel) represents densitometric analyses of the gel images to illustrate the ratio of phosphorylated protein to total protein in the absence (shaded bars) or presence (black bars) of ectopically expressed PTP1B.

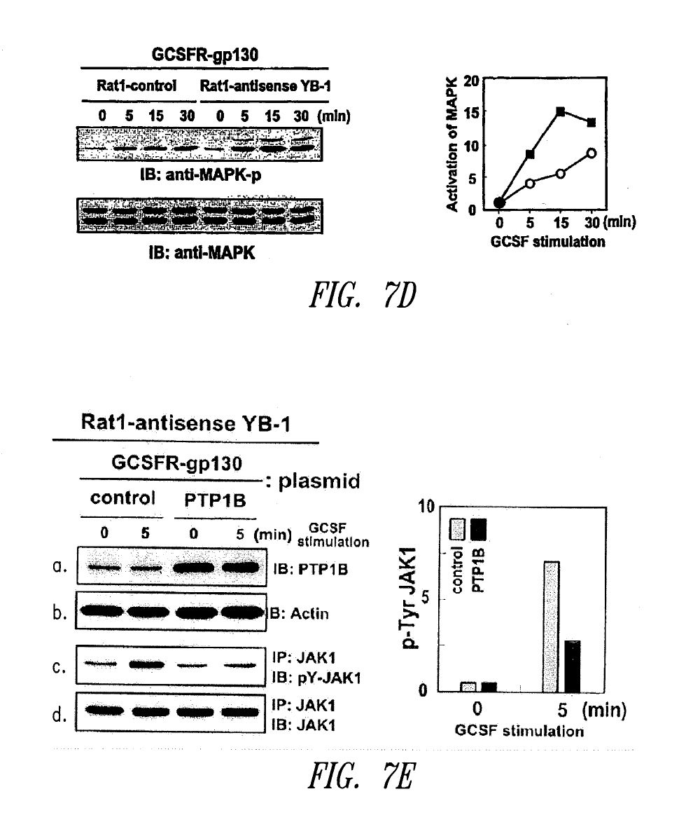

The effect of cytokine stimulation on cells that have diminished expression of YB-1 was also determined. Control Rat1 cells (Rat1-control) and Rat1 cells stably transfected with YB-1 antisense construct (clone number 20) (Rat1-antisense YB-1) were transiently transfected with chimeric receptor constructs (10 μg) comprising the extracellular domain of the G-CSF receptor, and the transmembrane and cytoplasmic domains of gp130 (Fukada et al., Immunity 5:449-60 (1996)). Cells were starved of serum for 6 hours and then stimulated with G-CSF (50 ng/ml) (Calbiochem) for 5, 15, or 30 minutes (see FIG. 7). Cells were then lysed and immunoprecipitation was performed as described above using either an anti-JAK1 antibody (1 μg) (BD Transduction Laboratories) or an anti-gp130 antibody (1 μg) (sc-655, Santa Cruz Biotechnology). The Jak-1 immunoprecipitates were immunoblotted using either an anti-phospho-JAK1 antibody (BioSource International, Camarillo, Calif.) or the anti-JAK1 antibody (FIG. 7A). The gp-130 immunoprecipitates were immunoblotted with either anti-phospho-tyrosine antibodies (anti-pY) (see above) or the anti-gp-130 antibody (FIG. 7B). In addition, whole cell lysates were separated by SDS-PAGE (20 μg per lane) and immunoblotted with either an anti-phospho-Tyr705-STAT3 antibody (Cell Signaling) or an anti-STAT3 antibody (sc-482, Santa Cruz Biotechnology) (FIG. 7C), or with either an anti-phospho-MAPK(Erk1/2) antibody (Cell Signaling) or an anti-MAPK(Erk1/2) antibody (Cell Signaling) (FIG. 7D).

In a similar manner to the experiment described above, the effect of forced re-expression of YB-1 in Rat1-antisense YB-1 cells (clone #20) stimulated by G-CSF was examined. Rat1-antisense YB-1 cells were transiently co-transfected with either the PTP1B expression plasmid (10 μg) or the pMT2 empty vector control (10 μg) and the recombinant construct expressing the G-CSFR-gp130 receptor (10 μg) (see above). Cells were starved of serum for 6 hours and then stimulated with G-CSF (50 ng/ml) for 5 minutes. Total cell lysate (20 μg) was subjected to SDS-PAGE followed by immunoblotting with antibodies specific for PTP1B and actin (control). In addition, JAK1 was immunoprecipitated from the cell lysate (see above) and immunoblotted with either the anti-phospho-JAK1 antibody or the anti-JAK1 antibody. The results of the experiments are presented in FIG. 7E. The graph (FIG. 7E, right panel) represents densitometric analyses of the gel images to illustrate the ratio of phosphorylated protein to total protein in the absence (shaded bars) or presence (black bars) of ectopically expressed PTP1B.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. An isolated polynucleotide comprising a nucleotide sequence as set forth in SEQ ID NO:1, or a complementary sequence thereto.

2. An isolated polynucleotide comprising a nucleotide sequence as set forth in SEQ ID NO: 1 or a variant thereof, or a complementary sequence thereto.

3. An isolated antisense polynucleotide comprising at least 20 consecutive nucleotides complementary to a polynucleotide according to claim 1.

4. An isolated polynucleotide comprising at least 15 consecutive nucleotides that is capable of hybridizing under moderately stringent conditions to a nucleotide sequence as set forth in SEQ ID NO:1, or a complementary sequence thereto.

5. A recombinant nucleic acid construct comprising a polynucleotide according to any one of claims 1-4.

6. A vector comprising the recombinant nucleic acid construct according to claim 5.

7. A host cell transfected or transformed with the vector according to claim 6.

8. A recombinant nucleic acid construct comprising a nucleotide sequence as set forth in SEQ ID NO:1 that is operably linked to a reporter gene.

9. The recombinant nucleic acid construct of claim 8 wherein the reporter gene encodes a polypeptide selected from the group consisting of luciferase and chloramphenicol acetyl transferase.

10. A vector comprising the recombinant nucleic acid construct according to either claim 8 or claim 9.

11. A host cell transfected or transformed with the vector according to claim 10.

12. A method for treating a PTP1B associated disorder comprising administering to a subject an agent that impairs binding of a Y-box protein to a PTP1B promoter Y-box protein binding site.

13. The method of claim 12 wherein the PTP1B associated disorder is a metabolic disorder.

14. The method of claim 12 wherein the PTP1B associated disorder is selected from the group consisting of type 1 diabetes, type 2 diabetes and impaired glucose tolerance.

15. The method of claim 12 wherein the PTP1B associated disorder is obesity.

16. The method of claim 12 wherein the agent that impairs binding of a Y-box protein to a PTP1B promoter Y-box protein binding site comprises the polynucleotide of any one of claims 1-4.

17. The method of claim 12 wherein the agent that impairs binding of a Y-box protein to a PTP1B promoter Y-box protein binding site comprises an antisense polynucleotide, the antisense polynucleotide comprising at least 15 consecutive nucleotides complementary to a polynucleotide that encodes a YB-1 protein.

18. The method of claim 17 wherein the YB-1 protein comprises an amino acid sequence as set forth in FIG. 3D and SEQ ID NO:2.

19. The method of claim 17 wherein the YB-1 protein comprises an amino acid sequence as set forth in, or encoded by, a sequence selected from the group consisting of GENBANK Accession No. J03827 [SEQ ID NO:4]; GENBANK Accession No. NP_{—}113751 [SEQ ID NO:51]; GENBANK Accession No.M57299 [SEQ ID NO:52; SEQ ID NO:53]; GENBANK Accession No. AAB46889.2 [SEQ ID NO:58]; GENBANK Accession No. AAA35750.1 [SEQ ID NO:54]; GENBANK Accession No. AAA75476.1 [SEQ ID NO:55]; GENBANK Accession No. AAA63390.1 [SEQ ID NO:56]; and GENBANK Accession No. BAA02569.1 [SEQ ID NO:57].

20. The method of claim 17, wherein the antisense polynucleotide comprises at least 20 consecutive nucleotides of a polynucleotide selected from the group consisting of a polynucleotide having a sequence as set forth in, or capable of encoding, SEQ ID NO:3, GENBANK Accession No. J03827 [SEQ ID NO:4], GENBANK Accession No. NP_{—}113751 [SEQ ID NO:51]; GENBANK Accession No.M57299 [SEQ ID NO:52; SEQ ID NO:53]; GENBANK Accession No. AAB46889.2 [SEQ ID NO:58]; GENBANK, GENBANK Accession No. AAA35750.1 [SEQ ID NO:54]; GENBANK Accession No. AAA75476.1 [SEQ ID NO:55]; GENBANK Accession No. AAA63390.1 [SEQ ID NO:56]; and GENBANK Accession No. BAA02569.1 [SEQ ID NO:57], or a complementary sequence thereto.

21. The method of claim 12 wherein the agent that impairs binding of a Y-box protein to a PTP1B promoter Y-box protein binding site comprises a polypeptide.

22. The method of claim 21 wherein the polypeptide comprises an antibody that specifically binds to the Y-box protein.

23. The method of claim 22 wherein the antibody is an intracellular antibody.

24. The method of claim 12 wherein the agent that impairs binding of a Y-box protein to a PTP1B promoter Y-box protein binding site is a small molecule.

25. The method of claim 12 wherein the PTP1B promoter Y-box protein binding site comprises a polynucleotide having a nucleotide sequence as set forth in SEQ ID NO:1 or a complement thereto.

26. The method of claim 12 wherein the PTP1B promoter Y-box protein binding site comprises a polynucleotide that is capable of hybridizing under moderately stringent conditions to a nucleotide sequence as set forth in SEQ ID NO:1, or a complementary sequence thereto.

27. A method for treating a PTP1B associated disorder comprising administering to a subject a pharmaceutical compositions that comprises an agent which impairs binding of a Y-box protein to a PTP1B promoter Y-box protein binding site and a suitable carrier.

28. A method for impairing binding of a Y-box polypeptide to a PTP1B promoter Y-box polypeptide binding site, comprising contacting a cell which comprises a Y-box polypeptide and a PTP1B promoter Y-box polypeptide binding site with a polynucleotide according to any one of claims 1-4.

29. The method of claim 28 wherein the step of contacting a cell is performed in vitro.

30. A method of identifying an agent that is capable of altering PTP1B expression comprising:
(a) contacting, in the absence and presence of a candidate agent, a sample comprising a Y-box protein and a recombinant nucleic acid construct that comprises a nucleotide sequence as set forth in SEQ ID NO:1 which is operably linked to a reporter gene, under conditions and for a time sufficient to detect transcription or expression of the reporter gene; and
(b) comparing a level of reporter gene transcription or expression in the absence of the candidate agent to a level of reporter gene transcription or expression in the presence of the candidate agent, wherein a decreased level of reporter gene transcription or expression in the presence of the candidate agent relative to the level of reporter gene transcription or expression in the absence of the candidate agent indicates the agent is capable of altering PTP1B expression.

31. The method of claim 30 wherein the sample comprises a cell.

32. The method of claim 30 wherein the sample comprises an isolated Y-box protein.

33. The method of claim 30 wherein the sample comprises an isolated recombinant nucleic acid construct.

34. The method of claim 30 wherein the Y-box protein comprises an amino acid sequence that is selected from the group consisting of the sequence set forth in SEQ ID NO:2 and the sequence set forth in SEQ ID NO:4.

35. The method of claim 30 wherein the reporter gene encodes a polypeptide selected from the group consisting of luciferase and chloramphenicol acetyl transferase.

36. A method of identifying an agent that is capable of altering PTP1B expression comprising:
(a) contacting a candidate agent and a biological sample comprising a cell that comprises a PTP1B gene and that is capable of PTP1B gene transcription or expression, under conditions and for a time sufficient to detect PTP1B gene transcription or expression; and
(b) comparing a level of PTP1B gene transcription or expression in the absence of the candidate agent to a level of PTP1B gene transcription or expression in the presence of the candidate agent, wherein a decreased level of PTP1B gene transcription or expression in the presence of the candidate agent relative to the level of PTP1B gene transcription or expression in the absence of the candidate agent indicates the agent is capable of altering PTP1B expression.

37. The method of claim 36 wherein the cell comprises an insulin receptor, further comprising determining a level of phosphorylation of the insulin receptor, wherein an increased level of insulin receptor phosphorylation in the presence of the candidate agent relative to the level of insulin receptor phosphorylation in the absence of the candidate agent indicates the agent is capable of altering PTP1B expression.

38. The method of claim 36 wherein the cell comprises an insulin receptor, further comprising determining a level of an insulin response in the cell, wherein an increased level of the insulin response in the presence of the candidate agent relative to the level of the insulin response in the absence of the candidate agent indicates the agent is capable of altering PTP1B expression.

39. The method of claim 38 wherein the insulin response is selected from the group consisting of glucose uptake, glycogen synthesis, lipogenesis, lipolysis, Glut4 recruitment to a plasma membrane and amino acid import.

40. The method of claim 38 wherein the insulin response is selected from the group consisting of insulin receptor tyrosine phosphorylation, MAP kinase phosphorylation, AKT phosphorylation, inhibition of phosphoenolpyruvate carboxykinase transcription and phosphatidylinositoltriphosphate kinase activation.

41. The method of claim 36 wherein the cell comprises a leptin receptor, further comprising determining a level of a leptin response in the cell, wherein an increased level of the leptin response in the presence of the candidate agent relative to the level of the leptin response in the absence of the candidate agent indicates the agent is capable of altering PTP1B expression.

42. The method of claim 41 wherein the leptin response is selected from the group consisting of TYK2 phosphorylation, JAK2 phosphorylation, STAT1 phosphorylation and STAT3 phosphorylation.
